(19) United States
(12) Patent Application Publication
(10) Pub. No.: US 2022/0403452 A1
(43) Pub. Date: Dec. 22, 2022

(54) SYSTEMS AND METHODS FOR BARCODING CELLS AND CELL BEADS

(71) Applicant: 10x Genomics, Inc., Pleasanton, CA (US)

(72) Inventors: Shea T. Lance, Livermore, CA (US); Geoffrey McDermott, Livermore, CA (US); Paul William Wyatt, Pleasanton, CA (US); Daniel P. Riordan, Pleasanton, CA (US); Michael Schnall-Levin, San Francisco, CA (US)

(21) Appl. No.: 17/687,376

(22) Filed: Mar. 4, 2022

Related U.S. Application Data
(63) Continuation of application No. PCT/US2020/049575, filed on Sep. 4, 2020.
(60) Provisional application No. 62/897,181, filed on Sep. 6, 2019.

(51) Int. Cl. C12Q 1/6834; C12Q 1/6806

(52) U.S. Cl. CPC C12Q 1/6834; C12Q 1/6806

(57) ABSTRACT

Provided are methods for profiling cellular analytes of a cell by barcoding the cell in a combinatorial split and pool iterative process. In some instances, a cell bead may be generated from the cell, and the analytes therein barcoded in a combinatorial split and pool iterative process while the analytes are retained in the cell bead during iterative partitioning.

### CROSS REFERENCE

This application is a continuation of International Application No. PCT/US2020/049575, filed Sep. 4, 2020, which claims the benefit of U.S. Provisional Application No. 62/897,181, filed Sep. 6, 2019, which applications are incorporated herein by reference in their entireties.

### BACKGROUND

A sample may be processed for various purposes, such as identification of a type of moiety within the sample. The sample may be a biological sample. Biological samples may be processed, such as for detection of a disease (e.g., cancer) or identification of a particular species. There are various approaches for processing samples, such as polymerase chain reaction (PCR) and sequencing.

Biological samples may be processed within various reaction environments, such as partitions. Partitions may be wells or droplets. Droplets or wells may be employed to process biological samples in a manner that enables the biological samples to be partitioned and processed separately. For example, such droplets may be fluidically isolated from other droplets, enabling accurate control of respective environments in the droplets.

Biological samples in partitions may be subjected to various processes, such as chemical processes or physical processes. Samples in partitions may be subjected to heating or cooling, or chemical reactions, such as to yield species that may be qualitatively or quantitatively processed.

### SUMMARY

Partition-based single cell sequencing technologies retain cell-of-origin information about intracellular analytes by physically separating cells into different compartments then applying a cell-specific molecular barcode to the analytes of interest. However, the Poisson statistics of cell capture needed to ensure that mostly single cells are isolated during encapsulation into the partitions means that the number of droplets and the pool of molecular barcodes required are substantially larger than the number of cells. For example, a partitioning output may include a (desired) subset of partitions comprising a single cell and molecular barcodes, and another (undesired) subset of partitions comprising molecular barcodes but no cells, resulting in the waste of valuable resources in the latter sub set.

To overcome this issue, in the in situ or “split-pool” barcoding approach, cells themselves are used as containers and intracellular analytes of the cells are molecularly labeled using combinatorial indexing. In practice, fixed cells are split into different wells of a microwell plate where a well-specific barcode is then appended to intracellular analytes entrapped within the fixed cells. Cells are then pooled back together. By repeating this process several times, the cells go through a unique combination of wells such that the corresponding intracellular analytes contain a combination of well-specific barcodes indicating their cell of origin. However, for every round of split-pooling, assay sensitivity can be impacted by diffusion of intracellular analytes of interest out of their fixed cell environment. An inherent reduction in intracellular concentration of analytes can also occur during each round of washing and pooling of cells.

To address the shortcomings of partition and split-pool based barcoding approaches, this invention disclosure describes an approach that combines the principles of both techniques. The present invention generally relates to a combination of molecular barcoding and emulsion-based microfluidics to isolate, lyse, and barcode intracellular analytes from individual cells in a high-throughput manner by barcoding the cells.

The methods provided herein allow for profiling of intracellular analytes from single cells quickly and inexpensively. To do so, using a split-pool method for combinatorial labeling, individual cells are first molecularly indexed on their outer surface with a composite barcode. Individual cells which are molecularly indexed are encapsulated in partitions (e.g., droplets, wells), where they are lysed. The molecular barcodes released from their surfaces are then used to tag intracellular analytes contained within the cell. Each cell may be uniquely barcoded so that each cell and its contents are distinguishable from other cells and their contents.

In other aspect, provided are methods for iteratively barcoding the intracellular analytes of singe cells at high throughput by utilizing cell beads that can encapsulate cells and physically retain the intracellular analytes between serial partitioning and barcoding processes of these cell beads, and overloading the cell beads during each partitioning iteration. At each iteration, the cell beads may be split into different partitions, partition-specific barcodes delivered to the cell beads to barcode intracellular analytes encapsulated by the cell beads, and then pooled back together. At each iteration of partitioning, cell beads may be overloaded to enable processing of multiple cells per partitioning at greater throughput then can be usefully achieved with normal sub-Poisson cell loading. That is, in a partition, more than one cell-bead may be present. After n iterations, the intracellular analytes of each cell bead may comprise a unique combination of barcodes that uniquely identifies the cell of origin from a pool of source cells.

In some instances, the cell beads themselves may be barcoded in accordance with methods for barcoding cells, as described herein. In some instances, the methods for barcoding cells may additionally overload cells during iterative partitioning operations to increase throughput.

In an aspect, also provided herein are methods of processing cells, comprising: (a) partitioning a plurality of cells and a plurality of nucleic acid barcode molecules comprising barcode sequences into a plurality of partitions, wherein a partition of the plurality of partitions comprises a first cell of the plurality of cells and a first barcode molecule of the plurality of nucleic acid barcode molecules, wherein the first barcode molecule comprises a first barcode sequence that is unique to the partition among the plurality of partitions; (b) in the partition, attaching the first barcode molecule to a surface of the first cell, wherein the first barcode sequence is different than other barcode sequences in other partitions of the plurality of partitions; (c) pooling cells from the plurality of cells, including the first cell, from the plurality of partitions; (d) re-partitioning the plurality of cells and an additional plurality of nucleic acid barcode molecules into an additional plurality of partitions, wherein a partition of the additional plurality of partitions comprises the first cell and an additional barcode molecule comprising an additional barcode sequence that is unique to the partition of the additional plurality of partitions; (e) in the partition of (d), coupling the additional barcode molecule to the first barcode molecule, thereby indexing the first cell with a nucleic acid composite barcode molecule comprising a composite barcode sequence comprising the first barcode sequence and additional barcode sequence, wherein the nucleic acid composite barcode molecule comprises a capture sequence configured to capture an analyte.

In some instances, subsequent to (e), (c)-(e) are repeated N times, wherein N is an integer greater than or equal to 1, and wherein the composite barcode sequence comprises the first barcode sequence and N+1 additional barcode sequences. The method of claim 2, wherein a (N+1)th barcode molecule is configured to couple to a Nth barcode nucleic acid molecule. In some instances, the method further comprises, prior to (a), coupling a cell coupling agent to the surface of the first cell, wherein the cell coupling agent is coupled to an oligonucleotide configured to couple to the first barcode molecule.

In some instances, prior to (a), a plurality of cell coupling agents are coupled to the surface of the first cell, wherein the plurality of cell coupling agents comprises the cell coupling agent. In some instances, the first barcode molecule is configured to couple to a second barcode molecule.

In some instances, the first barcode molecule is configured to couple to one or more splint molecules, wherein the one or more splint molecules are configured to couple to the second barcode molecule. In some instances, the cell coupling agent comprises a peptide or polypeptide.

In some instances, the peptide or polypeptide is configured to couple to an antigen on the cell surface of the first cell. In some instances, the peptide or polypeptide is configured to couple to a carbohydrate group on a cell membrane of the first cell. In some instances, the cell coupling agent comprises a lipid molecule, wherein the lipid molecule is configured to embed into a cell membrane of the first cell and the oligonucleotide is configured to couple to the first barcode molecule.

In some instances, the cell coupling agent comprises a disulfide bond. In some instances, the method further comprises, subsequent to indexing the first cell with the nucleic acid composite barcode molecule comprising the composite barcode sequence, partitioning the first cell into a third partition.

In some instances, the method further comprises coupling the nucleic acid composite barcode molecule comprising the composite barcode sequence to the analyte, wherein the analyte is a cellular analyte of the first cell, thereby generating a barcoded analyte. In some instances, the method further comprises, determining a sequence of the barcoded analyte, wherein the determined sequence of the barcoded analyte comprises the composite barcode sequence or complement thereof. In some instances, the method further comprises, using the composite barcode sequence or complement thereof to identify the analyte as a cellular analyte of the first cell

In some instances, the method further comprises, lysing the cell in the third partition to release the analyte. In some instances, the analyte is selected from a ribonucleic acid (RNA) molecule, a DNA molecule, a gDNA molecule, a protein, or any combination thereof In some instances, the RNA molecule is a messenger RNA (mRNA) molecule. In some instances, the method further comprises, releasing the cell coupling agent from the cell surface or releasing the oligonucleotide from the cell coupling agent.

In some instances, the releasing the cell coupling agent comprises cleaving a disulfide bond. In some instances, the partition is a droplet. In some instances, the partition is a well.

In some instances, the partition is a microwell or a nanowell.

In some instances, the partition is the nanowell, wherein the nanowell is from a nanowell array. In some instances, the microwell is from a 96-well plate or a 384-well plate. In some instances, subsequent to (a), the partition comprises more than one cell. In some instances, subsequent to (e), (c)-(e) are repeated 2 times, and wherein in (d) the additional plurality of partitions comprises at least 96 partitions. In some instances, subsequent to (e), (c)-(e) are repeated 3 times.

In some instances, (a)-(e) are performed for each cell of the plurality of cells, and wherein subsequent to (e), at least 99% of respective cells of the plurality of cells each comprises a respective composite barcode sequence that is unique to the respective cells among the plurality of cells.

In another aspect, further provided are methods of cellular processing, comprising: (a) contacting a cell with a cell coupling agent coupled to an oligonucleotide molecule, thereby generating a cell coupled to the coupling agent; (b) partitioning (i) the cell coupled to the coupling agent and (ii) a first barcode nucleic acid molecule comprising a first barcode sequence into a partition, and attaching the first barcode nucleic acid molecule to the oligonucleotide molecule; (c) pooling the cell coupled to the coupling agent with a plurality of cells; (d) partitioning (i) the cell coupled to the coupling agent and (ii) a second barcode nucleic acid molecule comprising a second barcode sequence into a second partition, and attaching the second nucleic acid barcode molecule to the first barcode nucleic acid molecule to generate a nucleic acid composite barcode molecule comprising the first barcode sequence and the second barcode sequence, wherein, subsequent to (d), the nucleic acid composite barcode molecule comprises a capture sequence configured to capture an analyte.

In some instances, subsequent to (d), (b)-(d) are repeated N times, wherein N is an integer greater than or equal to 1, and wherein the nucleic acid composite barcode molecule comprises the first barcode sequence and N additional barcode sequences.

In some instances, a Nth barcode sequence is configured to attach to a (N−1)th barcode sequence of the nucleic acid composite barcode molecule. In some instances, a Nth barcode nucleic acid molecule comprises a Nth barcode sequence and the capture sequence.

In some instances, the analyte is a genomic deoxyribonucleic acid (gDNA) molecule. In some instances, the analyte is a ribonucleic acid (RNA) molecule. In some instances, the RNA molecule is a messenger RNA molecule (mRNA). In some instances, the RNA molecule is (i) a clustered regularly interspaced short palindromic (CRISPR) RNA molecule (crRNA) or (ii) a single guide RNA (sgRNA) molecule. In some instances, the analyte is a protein. In some instances, the partition is a droplet.

In some instances, the partition is a well. In some instances, the method comprises performing (a)-(d) for the plurality of cells. In some instances, the first barcode nucleic acid molecule is attached to a first bead and/or the second barcode nucleic acid molecule is attached to a second bead.

In some instances, the cell coupling agent comprises a disulfide bond. In some instances, the method further comprises, partitioning the cell into a third partition. In some instances, the method further comprises, coupling the nucleic acid composite barcode molecule to the analyte, wherein the analyte is a cellular analyte of the cell. In some instances, the cell coupling agent comprises a moiety group that is a peptide or polypeptide. In some instances, the peptide or polypeptide is configured to couple to an antigen on the cell surface of the cell. In some instances, the peptide or polypeptide is configured to couple to a carbohydrate group on a cell membrane of the cell. In some instances, the cell coupling agent comprises a lipid molecule, wherein the lipid molecule is configured to embed into a cell membrane of the cell and the oligonucleotide is configured to couple to the first barcode molecule.

In an aspect are also systems, comprising: a plurality of partitions comprising a plurality of cells, wherein the plurality of cells comprises a plurality of nucleic acid barcode molecules coupled thereto, wherein a partition of the plurality of partitions comprises (i) a cell of the plurality of cells, wherein the cell comprises a nucleic acid barcode molecule of the plurality of nucleic acid barcode molecules coupled to a surface of the cell, wherein the barcode molecule comprises a barcode sequence unique to the cell among the plurality of cells, (ii) a nucleic acid molecule comprising a capture sequence configured to capture an analyte, and (iii) reagents configured to couple the nucleic acid molecule to the nucleic acid barcode molecule to generate a composite barcode molecule comprising the barcode sequence and the capture sequence.

In some instances, the reagents comprise a splint molecule configured to couple to each of the nucleic acid barcode molecule and the nucleic acid molecule. In some instances, the plurality of partitions are a plurality of droplets. In some instances, the plurality of partitions are a plurality of wells. In some instances, the partition is a microwell or a nanowell. In some instances, the partition is the nanowell, wherein the nanowell is from a nanowell array. In some instances, the microwell is from a 96-well plate or a 384-well plate. In some instances, the partition comprises more than one cell. In some instances, the nucleic acid barcode molecule is coupled to the surface of the cell via a cell coupling agent. In some instances, the cell coupling agent comprises a peptide or polypeptide.

In some instances, the peptide or polypeptide is coupled to an antigen on the surface of the cell. In some instances, the peptide or polypeptide is coupled to a carbohydrate group on a cell membrane of the cell. In some instances, the cell coupling agent comprises a lipid molecule, wherein the lipid molecule is embedded into a cell membrane of the cell.

In some instances, the cell coupling agent comprises a disulfide bond. In some instances, the capture sequence comprises a poly-T sequence. In some instances, the capture sequence comprises a template switching oligonucleotide sequence. In some instances, the capture sequence comprises a poly-G sequence.

In another aspect, provided are compositions, comprising: a plurality of cells comprising a plurality of nucleic acid barcode molecules coupled thereto, wherein a cell of the plurality of cells comprises a nucleic acid barcode molecule of the plurality of nucleic acid barcode molecules coupled to a surface of the cell, wherein the nucleic acid barcode molecule comprises (i) a barcode sequence unique to the cell among the plurality of cells, and (ii) a capture sequence configured to capture an analyte.

In some instances, the plurality of cells are provided in bulk solution. In some instances, the plurality of cells are provided in a plurality of partitions. In some instances, the plurality of partitions are a plurality of droplets. In some instances, the plurality of partitions are a plurality of wells. In some instances, the plurality of partitions are microwells or nanowells. In some instances, the plurality of partitions are nanowells in a nanowell array. In some instances, the plurality of partitions are microwells from a 96-well plate or a 384-well plate. In some instances, a partition of the plurality of partitions comprises the cell. In some instances, the nucleic acid barcode molecule is coupled to the surface of the cell via a cell coupling agent. In some instances, the cell coupling agent comprises a peptide or polypeptide. In some instances, the peptide or polypeptide is coupled to an antigen on the surface of the cell. In some instances, the peptide or polypeptide is coupled to a carbohydrate group on a cell membrane of the cell. In some instances, the cell coupling agent comprises a lipid molecule, wherein the lipid molecule is embedded into a cell membrane of the cell. In some instances, the cell coupling agent comprises a disulfide bond. In some instances, the capture sequence comprises a poly-T sequence. In some instances, the capture sequence comprises a template switching oligonucleotide sequence.

In some instances, the capture sequence comprises a poly-G sequence. In some instances, the plurality of partitions of (a) and the additional plurality of partitions of (d) are from a same set of partitions. In some instances, the plurality of partitions of (a) and the additional plurality of partitions of (d) are from different sets of partitions.

In an aspect, also provided are methods of cellular analysis, comprising: (a) generating a plurality of cell beads from a plurality of cells, wherein the plurality of cell beads is configured to physically retain an analyte derived from a cell in the cell bead; (b) partitioning the plurality of cell beads and a plurality of nucleic acid barcode molecules comprising barcode sequences into a plurality of partitions, wherein a partition of the plurality of partitions comprises two or more cell beads, including a first cell bead, and nucleic acid barcode molecules comprising a first barcode sequence, wherein the first barcode sequence is different than other barcode sequences in other partitions of the plurality of partitions; (c) in the partition, attaching the first barcode sequence to an analyte derived from the first cell bead; (d) pooling cell beads from the plurality of cell beads, including the first cell bead, from the plurality of partitions; and (e) performing (b)-(d) N times to introduce N number of different barcode sequences to the first cell bead, wherein N is an integer greater than or equal to 2, to generate a composite barcode

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of a microfluidic channel structure for partitioning individual biological particles.

FIG. 2 shows an example of a microfluidic channel structure for delivering barcode carrying beads to droplets.

FIG. 3 shows an example of a microfluidic channel structure for co-partitioning biological particles and reagents.

FIG. 4 shows an example of a microfluidic channel structure for the controlled partitioning of beads into discrete droplets.

FIG. 5 shows an example of a microfluidic channel structure for increased droplet generation throughput.

FIG. 6 shows another example of a microfluidic channel structure for increased droplet generation throughput.

FIG. 7A shows a cross-section view of another example of a microfluidic channel structure with a geometric feature for controlled partitioning. FIG. 7B shows a perspective view of the channel structure of FIG. 7A.

FIG. 8 illustrates an example of a barcode carrying bead.

FIG. 9 illustrates an example of a barcode carrying cell.

FIG. 10 shows an example of a microfluidic channel structure for partitioning individual surface-barcoded cells for analysis of intracellular analytes.

FIG. 11 shows the split-pool workflow for generation of surface-barcoded cells.

FIG. 12 shows a computer system that is programmed or otherwise configured to implement methods provided herein.

FIG. 13 illustrates a process flow of iterative cell bead barcoding.

FIG. 14 schematically illustrates an example microwell array.

FIG. 15 schematically illustrates an example workflow for processing nucleic acid molecules.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The term “barcode,” as used herein, generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads.

The term “real time,” as used herein, can refer to a response time of less than about 1 second, a tenth of a second, a hundredth of a second, a millisecond, or less. The response time may be greater than 1 second. In some instances, real time can refer to simultaneous or substantially simultaneous processing, detection or identification.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses).

The term “genome,” as used herein, generally refers to genomic information from a subject, which may be, for example, at least a portion or an entirety of a subject's hereditary information. A genome can be encoded either in DNA or in RNA. A genome can comprise coding regions (e.g., that code for proteins) as well as non-coding regions. A genome can include the sequence of all chromosomes together in an organism. For example, the human genome ordinarily has a total of 46 chromosomes. The sequence of all of these together may constitute a human genome.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

As used herein, the term “barcoded nucleic acid molecule” generally refers to a nucleic acid molecule that results from, for example, the processing of a nucleic acid barcode molecule with a nucleic acid sequence (e.g., nucleic acid sequence complementary to a nucleic acid primer sequence encompassed by the nucleic acid barcode molecule). The nucleic acid sequence may be a targeted sequence or a non-targeted sequence. For example, in the methods and systems described herein, hybridization and reverse transcription of a nucleic acid molecule (e.g., a messenger RNA (mRNA) molecule) of a cell with a nucleic acid barcode molecule (e.g., a nucleic acid barcode molecule containing a barcode sequence and a nucleic acid primer sequence complementary to a nucleic acid sequence of the mRNA molecule) results in a barcoded nucleic acid molecule that has a sequence corresponding to the nucleic acid sequence of the mRNA and the barcode sequence (or a reverse complement thereof). A barcoded nucleic acid molecule may serve as a template, such as a template polynucleotide, that can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the mRNA.

The term “sample,” as used herein, generally refers to a biological sample of a subject. The biological sample may comprise any number of macromolecules, for example, cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The term “biological particle,” as used herein, generally refers to a discrete biological system derived from a biological sample. The biological particle may be a macromolecule. The biological particle may be a small molecule. The biological particle may be a virus. The biological particle may be a cell or derivative of a cell. The biological particle may be an organelle. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell, but may not include other constituents of the cell. An example of such constituents is a nucleus or an organelle. A cell may be a live cell. The live cell may be capable of being cultured, for example, being cultured when enclosed in a gel or polymer matrix, or cultured when comprising a gel or polymer matrix.

The term “macromolecular constituent,” as used herein, generally refers to a macromolecule contained within or from a biological particle. The macromolecular constituent may comprise a nucleic acid. In some cases, the biological particle may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “partition,” as used herein, generally, refers to a space or volume that may be suitable to contain one or more species or conduct one or more reactions. A partition may be a physical compartment, such as a droplet or well. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

The term “coupled to,” as used herein, generally refers to a physical association between objects. The physical association may be reversible or substantially irreversible. For example, the physical association may be temporary or substantially permanent. For example, a first object coupled to a second object may be releasable from the first object. In some cases, the first object can be released from the second object, or vice versa, upon application of a stimulus, which stimulus may comprise a photostimulus (e.g., ultraviolet light), a thermal stimulus, a chemical stimulus (e.g., reducing agent), or any other useful stimulus. In an example, a nucleic acid molecule is coupled to a particle or support (e.g., bead). In another example, a nucleic acid molecule is coupled to another nucleic acid molecule. Coupling may encompass immobilization (e.g., as described herein). Similarly, coupling may encompass attachment, such as attachment. Coupling may comprise any interaction that affects a physical association between two objects, including, for example, a covalent bond, a non-covalent interaction (e.g., electrostatic interaction [e.g., hydrogen bonding, ionic interaction, and halogen bonding], π-interaction [e.g., π-π interaction, polar-π interaction, cation-π interaction, and anion-π interaction], van der Waals force-based interactions [e.g., dipole-dipole interactions, dipole-induced dipole interactions, and induced dipole-induced dipole interactions], hydrophobic interaction), a magnetic interaction (e.g., magnetic dipole-dipole interaction, indirect dipole-dipole coupling), an electromagnetic interaction, adsorption, or any other useful interaction. For example, a nucleic acid molecule may be coupled to a support via a covalent interaction or non-covalent interaction. A coupling between a first object and a second object may comprise a labile moiety, such as a moiety comprising an ester, vicinal diol, phosphodiester, peptidic, glycosidic, sulfone, Diels-Alder, or similar linkage. The strength of a coupling between a first object and a second object may be indicated by a dissociation constant, Kd, that indicates the inclination of a coupled object comprising a first object and a second object to dissociate into the uncoupled first and second objects, and may be expressed as a ratio of dissociated (e.g., uncoupled) objects to coupled objects. A smaller dissociation constant is generally indicative of a stronger coupling between coupled objects. In some cases, coupled objects may exist in dynamic equilibrium with the corresponding uncoupled components. Where a first object is coupled to a second object, the first object may be coupled directly to the second object, via an interaction of a component of the first object (e.g., a first sequence segment) and a component of the second object (e.g., a second sequence segment). Alternatively or in addition, a first object may be coupled indirectly to the second object via an interaction of one or both of the first object and the second object with one or more intermediary objects, such as an additional molecule. In an example, a first nucleic acid molecule is coupled to a second nucleic acid molecule via a splint molecule.

In some cases, coupled objects and their corresponding uncoupled components may exist in dynamic equilibrium with one another. For example, a solution comprising a plurality of coupled objects each comprising a first object and a second object may also include a plurality of first objects and a plurality of second objects, unassociated with each other. At a given point in time, a given first object and a given second object may be coupled to one another or the objects may be uncoupled; the relative concentrations of coupled and uncoupled components throughout the solution will depend upon the strength of the coupling between the first and second objects (reflected in the dissociation constant).

### Molecular Barcoding of Cells and Cell Beads

The methods provided herein, in some embodiments, allow for profiling of intracellular analytes from single cells quickly and inexpensively. Using a split-pool method for combinatorial labeling, individual cells may first be labeled, or molecularly indexed, on their outer surface with a composite barcode (e.g., comprising molecular indexes or molecular barcodes). Individual cells which are molecularly indexed may then be encapsulated in partitions (e.g., droplets, wells), where they can be lysed. The molecular barcodes released from their surfaces (e.g., prior to, during, or subsequent to such lysing) can then be used to tag intracellular analytes contained within the cell. Each cell may be uniquely barcoded so that it and its contents are distinguishable from other cells and their contents. The molecular barcodes on the outer surface of the cell may be generated using a split-pool combinatorial mechanism, with serial partitioning and pooling, as described elsewhere herein.

In other aspects, provided are methods for iteratively barcoding the intracellular analytes of single cells at high throughput by utilizing cell beads that can encapsulate cells and physically retain the intracellular analytes between serial partitioning and barcoding processes of these cell beads, and overloading the cell beads during each partitioning iteration. At each iteration, the cell beads may be split into different partitions, partition-specific barcodes delivered to the cell beads to barcode intracellular analytes encapsulated by the cell beads, and then pooled back together. At each iteration of partitioning, cell beads may be overloaded to enable processing of multiple cells per partition at greater throughput then can be usefully achieved with normal sub-Poisson cell loading. That is, in a partition, more than one cell-bead may be present. After n iterations, the intracellular analytes of each cell bead may comprise a unique combination of barcodes that uniquely identifies the cell of origin from a pool of source cells.

In some instances, the cell beads themselves may be barcoded in accordance with methods for barcoding cells, described herein. In some instances, the methods for barcoding cells may additionally overload cells during iterative partitioning operations to increase throughput.

Included within this disclosure are cell compositions that may include diverse sets of reagents, such as diverse libraries of cells attached to large numbers of oligonucleotides containing barcode sequences, and methods of making and using the same. The present disclosure provides methods, compositions, devices and kits for the generation of cells with covalent or non-covalent attachment of polynucleotides. Such cells may be used for any application, including analysis of the intracellular analytes.

This disclosure provides methods, systems, and compositions useful in the processing of sample materials through the controlled delivery of reagents to subsets of sample components, followed by analysis of those sample components employing, in part, the delivered reagents. In many cases, the methods and compositions are employed for processing of intracellular analytes and for nucleic acid analysis applications.

For example, certain aspects are directed to systems and methods for labeling cellular analytes within partitions, such as droplets or wells. In one set of embodiments, the cellular analytes and/or labels may comprise nucleic acids. In some cases, the labels may comprise ‘barcodes’ or unique sequences that can be used to distinguish analytes in a partition (e.g., droplet) from those in another partition, for instance after the partition contents are pooled together. In some cases, the unique sequences can be introduced into droplets using surface-indexed cells and attached to the analytes contained within the droplet (for example, released from lysed cells).

Certain aspects of the disclosure relate to systems and methods for containing or encapsulating analytes and oligonucleotide tags within microfluidic droplets or other suitable compartments (such as wells of a microwell array) and covalently bonding them together. In some cases, the analytes may arise from lysed cells and the methods may involve partitioning a cell with oligonucleotide tags, and then lysing the cell to release the analytes into the partition with the oligonucleotide tags. The oligonucleotide tags within a partition, such as a droplet (or attached to a cell) can be distinguishable from oligonucleotide tags in other partitions (or attached to other cells), e.g., within a plurality of partitions (or plurality of cells). For instance, the oligonucleotide tags can contain one or more unique sequences or barcodes that are different between the various partitions (or cells), and thus the analytes within each partition, such as a droplet (or cell) can be uniquely identified by determining the barcodes associated with the analyte.

Certain aspects of this disclosure relate to systems and methods of introducing oligonucleotide tags into the partitions by initially attaching them to a cell surface, than subsequently releasing them from the cell surface after the cell has been partitioned into a partition (e.g., droplet). It will be appreciated that while systems and methods described herein use “droplets” as examples of partitions, any other type of partition or suitable compartment, such as wells, may apply. The cells may be prepared such that most or all of the cells may contain only one set of uniquely distinguishable oligonucleotide tag, relative to other cells having other distinguishable oligonucleotide tags. If the cells are present within the droplets at a density of 1 cell/droplet (or less), then once the oligonucleotide tags are released from the cell surface, most or all of the droplets will contain one unique oligonucleotide tag or no oligonucleotide tag, thus allowing each droplet (and the analytes contained therein) to be uniquely labeled.

Certain aspects of this disclosure provide systems and methods for creating a surface-barcoded cell carrying an oligonucleotide tag (e.g., each surface bound oligonucleotide comprising a barcode, a primer, and/or other functional sequences suitable for, e.g., capture, amplification and/or sequencing of nucleic acids) coupled (e.g., covalently bound) on the surface of the cell. The cells may be prepared such that a given cell's surface can be decorated with only one uniquely distinguishable oligonucleotide tag, relative to other cells having other distinguishable oligonucleotide tags. In some cases, the surface-indexed cell is encapsulated into a droplet and the oligonucleotide tags are released from the surface of the cell. In some cases, the cell is lysed to release the contents of the cell and the unique oligonucleotides can be incorporated into the released cellular analytes. In some instances, cells can be present within the droplets at a density of 1 cell/droplet (or less), so that the unique oligonucleotide tags released from the cell can allow each droplet (and the analytes contained there) to be uniquely identified.

Certain aspects of this disclosure provide systems and methods for increasing throughput by overloading a partition with multiple cells or cell beads (e.g., per partition) during combinatorial barcode indexing of the cells or cell beads, or barcoding of contents (e.g., analytes) thereof.

### Surface Barcoding

In certain embodiments, provided herein are methods for the creation of a surface-barcoded cell or a surface-indexed cell, wherein the cell surface is modified to carry a plurality of nucleic acid molecules comprising a barcode sequence (also referred to as “oligonucleotide tags”). The cell surface may carry a high concentration of (e.g., 1 to 100 micromolar) oligonucleotide tags A given cell surface-associated oligonucleotide tag may comprise a composite barcode sequence, derived from a combination of at least 2 or more individual constituent barcode molecules, each comprising a partial barcode sequence. All or most of the nucleic acid fragments on a given surface-indexed cell may comprise the same composite barcode sequence.

The systems, methods, and kits described herein, may comprise or use a mixture comprising a plurality of cells whose respective surfaces carry a plurality of oligonucleotide tags. In some instances, an oligonucleotide tag comprising a composite barcode sequence can comprise one or more of: cell coupling agent, a linker molecule attaching the cell coupling agent to an oligonucleotide tag, and one or more functional sequences such as an adapter sequence, a primer or primer binding sequence (e.g., a sequencing primer or sequencing primer binding site), a unique molecular index (UMI), a sequence configured to attach to the flow cell of a sequencer (e.g., an Illumina P5, P7, or partial sequence thereof), a capture sequence configured to hybridize to a specific sequence or molecule (e.g., poly-T sequence configured to attach to a poly-A containing molecule, such as an mRNA), etc.

Methods of generating surface-indexed cells can generally include functionalizing the cell surface with a cell coupling agent that may be attached (1) directly to oligonucleotide sequences (e.g., comprising a universal adapter for partial barcode attachment) or (2) indirectly to oligonucleotide sequences through the use of linkers. In other embodiments, the oligonucleotide may be attached directly to the surface of the cell through a covalent bond. For example, in some instances, a cell is contacted with an agent (e.g. a chemical agent) that generates functional groups on the surface of a cell (e.g., on proteins on the surface of the cell). In some embodiments, a component on the surface of the cell (e.g., a surface protein or carbohydrate) is modified to comprise a —COOH (carboxylic acid), —NH₂ (amine), —CH₂Cl (chloromethyl), —CONH₂ (amide), —CONHNH₂ (hydrazide), —CHO (aldehyde) —OH (hydroxyl) —SH (thiol), —COC— (epoxy), click chemistry functional group, or maleimido group. An oligonucleotide (e.g., containing a universal adapter sequence as described elsewhere herein) can then be reacted with the functional group to attach the oligonucleotide to the cell surface. In some instances, the oligonucleotide comprises a functional group that reacts with the functional group on the cell surface. In some embodiments, the oligonucleotide to be conjugated to the cell surface comprises an amino modification, a thiol modification, or an acrydite modification. In some embodiments, the oligonucleotide and the cell surface are each functionalized with a click chemistry functional group (such as constituents of a copper-free click chemistry, e.g., SPAAC) as described in, e.g., Takayama Y, et al., Click Chemistry as a Tool for Cell Engineering and Drug Delivery; Molecules 2019, 24(1), 172. In some embodiments, chemical cross-linkers are utilized to couple the oligonucleotide to component on the cell surface. In some instances, commercial conjugation kits and chemistries (such as Lightning-Link®, Thunder-Link®, or Protein-Oligonucleotide Conjugation Kit (HyNic/4FB conjugation—Solulink) are utilized to attach the oligonucleotide to a component on the cell surface.

A cell coupling agent is an agent or molecule capable of associating with the surface of a cell. In some instances, the cell coupling agent may comprise a material, chemical, molecule, or moiety that is capable of attaching or binding to the surface of a cell or a component thereof or is capable of inserting into the plasma membrane. A cell coupling agent may also be capable of binding or attaching to a natural or modified nucleotide. Natural or modified oligonucleotides may be conjugated (covalent attachment by chemical and biological methods) to cell coupling agents, such as antibodies or their fragments, liposomal components, saccharides, hormones, proteins and peptides, toxins, fluorophores or photoprobes, inhibitors, enzymes, growth factors, and vitamins. The cell coupling agent may be a natural or synthetic ligand. The ligand may be a protein, polypeptide, carbohydrate, lipid or a combination thereof, which has functional groups that are exposed sufficiently to be recognized by a cell surface structure. The cell coupling agent may be a molecule which can insert or anchor into the lipid bilayer of cellular membranes. Cell coupling agents may be peptides, fatty acids, or other molecules which have an ability to insert into cellular plasma membranes. The cell coupling agent may also be a component of a biological organism such as a virus, cells (e.g., mammalian, bacterial, protozoan) or artificial carriers such as liposomes. Cell coupling agents may include nanoparticles, poly(lactic-co-glycolic acid) (PLGA) microspheres, lipidoids, lipoplex, liposome, carbohydrates (including simple sugars), cationic lipids, fibrin and derivatives of fibrin, polymers, thrombin, rapidly eliminated lipid nanoparticles (reLNPs) and combinations thereof. Various embodiments of cell coupling agents are described herein.

The cell coupling agent may be a natural or synthetic ligand which specifically binds a cellular surface structure. The cell coupling agent may also be a receptor or receptor-like molecule, such as an antibody or an analogue of an antibody, i.e., single chain antibody, which binds a cellular surface structure such as a ligand (e.g., antigen). The cell surface structure may be a receptor, e.g., the asialoglycoprotein receptor of hepatocytes. The cell coupling agent used may vary according to the target cell type to be indexed. For example, for hepatocytes, glycoproteins having exposed terminal carbohydrate groups such as asialoglyco-protein (galactose-terminal) can be used, although other ligands such as polypeptide hormones may also be employed. Other types of ligands may be used for targeting the molecular conjugate to cell-surface receptors, such as mannose for macrophages (lymphoma), mannose-6-phosphate glycoproteins for fibroblasts (fibrosarcoma), intrinsic factor-vitamin B12 for enterocytes and insulin for fat cells. The cell coupling agent may be a cell adhesion molecule or a functional fragment thereof. In some cases, the cell coupling agent may be an antibody to an adhesion molecule. Examples of an adhesion molecule include but are not limited to cadherins, immunoglobulin-like superfamily such as ICAM1, ICAM2, VCAM, integrins, etc.

The cell coupling agent (e.g., a cell coupling agent conjugated to an oligonucleotide “oligonucleotide conjugate”) may be selectively directed to target the oligonucleotide conjugate to an appropriate cell, using, e.g., a blocking agent. For example, the cell coupling agent may be in an inactive form, wherein the cell coupling agent may be masked by cleavably linking the cell coupling agent to a masking or blocking agent, thereby blocking the oligonucleotide-conjugate from inserting into the cellular plasma membrane or otherwise associated with a molecule on the cell surface. The blocking agent can be a bulky or an electrically charged moiety. It may be an amino acid, peptide or protein. The blocking agent may be an antibody or a ligand for a cell surface receptor, e.g., a viral antigen receptor, which can serve to target the oligonucleotide-conjugate to a desired cell or cell population in a plurality of cells. For example, monoclonal antibodies specific for tumor-associated cell surface antigens expressed on tumor cells or for virus-specific antigens expressed on the surface of virus-infected cells may be used to direct the oligonucleotide complex to a specific target cell in a given plurality of cells. See Pastan, I. et al. (1986) Cell 47:641-648; Vitetta, E. S. et al. (1987) Science 238:1098-1104, each of which are entirely incorporated herein by reference. Other examples are interleukin-2 which binds interleukin-2 receptors, and recombinant soluble CD4 antigen which binds to envelope glycoprotein gp120 of HIV-1 expressed on the surface of virions and HIV-1-infected cells. The blocking agent may be short peptides or amino acids. The short peptides may be Cys-Glu, or Cys-Glu-Glu. In addition, two or more cell coupling agents can be interlinked.

Cell coupling agents may be peptides that are capable of insertion into cellular membranes. Examples include fusogenic polypeptides such as peptidic segments from the fusion proteins of syncitia-forming viruses, ion-channel forming polypeptides and other peptides with affinity for membrane lipids. Another example is the hydrophobic C-terminal peptidic segment of cytochrome b5. Other examples include the transmembrane region of membrane bound IgE.

Attachment of the cell coupling agent (e.g., oligonucleotide-conjugate) to the cell surface may involve modulation of the cell surface pH. For example, acidity at the cell surface may be used in conjunction with the pH (low) insertion peptides (pHLIPs) where the insertion mechanism is triggered by the protonation of negatively charged residues of the peptide at low pH, leading to an increase of peptide's hydrohobicity thus shifting the equilibrium toward partitioning of the peptide into the bilayer. Lipid, polymer or metal based nanomaterials decorated with the pHLIP-oligonucleotide complex may be used as biocompatible nanocarriers for targeting to the cell surface. In some examples, liposomes and niosomes in conjunction with the phLIP-oligonucleotide complex may be directing the complex to the cell membrane. In some cases, pHLIP bundles (conjugates comprising two or more pHLIP peptides linked by polyethylene glycol) or Var3 pHLIPs comprising either the nonstandard amino acid, γ-carboxyglutamic acid, or a glycine-leucine-leucine motif may be complexed with the oligonucleotide to direct the complex to the cell membrane.

The cell coupling agent may be directed to the cell membrane using an Escherichia coli enzyme biotin ligase (BirA) that can sequence-specifically ligate a ketone isostere of biotin to a 15-amino-acid acceptor peptide (AP). AP-fused recombinant cell surface proteins may be tagged with the ketone probe and then specifically conjugated to a nucleic acid molecule functionalized with a hydrazide- or hydroxylamine moieties.

The cell coupling agent may be designed as a self-assembling peptide. Peptide amphiphile nanospheres decorated with cell surface binding peptide (e.g., KRSR) may be complexed with oligonucleotides for non-toxic delivery to the cell surface.

Peptide/oligonucleotide conjugates may be prepared by solid phase synthesis, or by solution phase conjugation of the peptide to the oligonucleotide followed by purification. A variety of linkages may be used for conjugation of peptides with oligonucleotides, including amide, thioether, thiol-maleimide, ester, and disulfide linkages. Peptides conjugates with non-charged oligonucleotides may be purified by reversed phase HPLC while conjugates with charged oligonucleotides may be purified by ion exchange or by large scale PAGE. See Juliano et al., Acc Chem Res. 2012 July 17; 45(7): 1067-76, which is entirely incorporated herein by reference.

A cell coupling agent may be a membrane-anchoring moiety such as a hydrophobic moiety that can dissolve in the hydrophobic core of the cellular membrane. The membrane-anchoring moiety of the membrane attachment complex may embed itself within the hydrophobic region of the membrane, leaving the oligonucleotide exposed as a projection extending outwardly from the surface. The hydrophobic anchoring moiety may be, for example, steroids, fatty acids, hydrophobic peptides and lipids. The hydrophobic anchoring moiety may be a cholesterol or a derivative thereof In some embodiments, the oligonucleotide (e.g. nucleic acid barcode molecule or barcoded oligonucleotide) is coupled to a lipophilic molecule (i.e. a cell coupling agent), and labeling cells in comprises delivering the nucleic acid barcode molecule to a cell membrane or a nuclear membrane by the lipophilic molecule (see, e.g. WO2019113533, which is entirely incorporated herein by reference) . Lipophilic molecules can insert into lipid membranes such as cell membranes and nuclear membranes. In some cases, the insertion can be reversible. In some cases, the nucleic acid barcode molecule or barcoded oligonucleotide comprising the nucleic acid barcode molecule can enter into the intracellular space and/or a cell nucleus. Non-limiting examples of lipophilic molecules that can be used in embodiments herein include sterol lipids such as cholesterol, tocopherol, and derivatives thereof, lignoceric acid, and palmitic acid. Other such exemplary lipophilic molecules comprise amphiphilic molecules wherein the headgroup (e.g., charge, aliphatic content, and/or aromatic content) and/or fatty acid chain length (e.g., C12, C14, C16, or C18) can be varied. For instance, fatty acid side chains (e.g., C12, C14, C16, or C18) can be coupled to glycerol or glycerol derivatives (e.g., 3-t-butyldiphenylsilylglycerol), which can also comprise, e.g., a cationic head group. The oligonucleotides (e.g. nucleic acid barcode molecules for cell-surface indexing) disclosed herein can then be couples (either directly or indirectly) to these amphiphilic molecules.

In some instances, an oligonucleotide (e.g. a nucleic acid barcode molecule) is attached to a lipophilic moiety (e.g., a cholesterol molecule). In some embodiments, the oligonucleotide (e.g. nucleic acid barcode molecule) is attached to the lipophilic moiety via a linker, such as a tetra-ethylene glycol (TEG) linker. Other exemplary linkers include, but are not limited to Amino Linker C6, Amino Linker C12, Spacer C3, Spacer C6, Spacer C12, Spacer 9, Spacer 18. In some instances, the oligonucleotide (e.g. a nucleic acid barcode molecule) is attached to the lipophilic moiety or the linker on the 5′ end of the nucleic acid barcode molecule. In some instances, the oligonucleotide (e.g. a nucleic acid barcode molecule) is attached to the lipophilic moiety or the linker on the 3′ end of the nucleic acid barcode molecule. In some instances, a first oligonucleotide (e.g. a nucleic acid barcode molecule) is attached to the lipophilic moiety or the linker at the 5′ end of the oligonucleotide (e.g. a nucleic acid barcode molecule) and a second oligonucleotide (e.g. a nucleic acid barcode molecule) is attached to the lipophilic moiety or the linker at the 3′ of the nucleic acid barcode molecule. The linker may be a glycol or derivative thereof. In some instances, the linker is tetra-ethylene glycol (TEG) or polyethylene glycol (PEG). In some instances, the oligonucleotide (e.g. a nucleic acid barcode molecule) is releasably attached to the linker or lipophilic moiety (e.g., as described elsewhere herein for releasable attachment of nucleic acid molecules) such that the oligonucleotide (e.g. a nucleic acid barcode molecule) or a portion thereof can be released from the lipophilic molecule.

In some instances, cells can be contacted with one or more additional agents along with moiety-conjugated oligonucleotides (e.g., the lipophilic molecules described herein). For example, in some embodiments, cells are contacted with a lipophilic moiety-conjugated barcode molecule and one or more additional moiety (e.g., lipophilic moiety) conjugated “anchor” molecules. In some instances, a cell is contacted with (1) a lipophilic-moiety conjugated to a first nucleic acid molecule comprising a capture sequence (e.g., a poly-A sequence), a barcode or indexing sequence, and a primer sequence; and (2) an anchor molecule comprising a lipophilic moiety conjugated to a second nucleic acid molecule comprising a sequence complementary to the primer sequence. In other instances, a cell is contacted with (1) a lipophilic-moiety conjugated to a first nucleic acid molecule comprising a capture sequence (e.g., a poly-A sequence), a barcode or indexing sequence, and a primer sequence; (2) an anchor molecule comprising a lipophilic moiety conjugated to a second nucleic acid molecule comprising an anchor sequence and a sequence complementary to the primer sequence; and (3) a co-anchor molecule comprising a lipophilic moiety conjugated to a third nucleic acid molecule comprising a sequence complementary to the anchor sequence. Moiety-conjugated oligonucleotides can comprise any number of modifications, such as modifications which prevent extension by a polymerase and other such modifications described elsewhere herein.

The structure of the moiety-attached barcode oligonucleotides may include a number of sequence elements in addition to the barcode or indexing sequence. The oligonucleotide may include functional sequences that are used in subsequent processing, which may include one or more of a sequencer specific flow cell attachment sequence, e.g., a P5 or P7 sequence for Illumina sequencing systems, as well as sequencing primer sequences, e.g., a R1 or R2 sequencing primer sequence for Illumina sequencing systems. A specific priming and/or capture sequence, such as poly-A sequence, may be also included in the oligonucleotide structure.

As described above, moiety-attached barcode oligonucleotides can be processed to attach a cell barcode sequence. In some embodiments, cell barcode oligonucleotides (which can be attached to a bead) comprise a poly-T sequence designed to hybridize and capture poly-A containing moiety-attached barcode oligonucleotides. In some embodiments, the poly-T cell barcode molecules comprise an anchoring sequence segment to ensure that the poly-T sequence hybridizes to the poly-A sequence of the moiety-attached barcode oligonucleotides. This anchoring sequence can include a random short sequence of nucleotides, e.g., 1-mer, 2-mer, 3-mer or longer sequence. An additional sequence segment may be included within the cell barcode oligonucleotide molecules. In some cases, this additional sequence provides a unique molecular identifier (UMI) sequence segment, e.g., as a random sequence (e.g., such as a random N-mer sequence) that varies across individual oligonucleotides (e.g., cell barcode molecules coupled to a single bead), whereas the cell barcode sequence is constant among the oligonucleotides (e.g., cell barcode molecules coupled to a single bead). This unique sequence may serve to provide a unique identifier of the starting nucleic acid molecule that was captured, in order to allow quantitation of the number of original molecules present (e.g., the number of moiety-conjugated nucleic acid barcode molecules).

For example, a cell coupling agent may be cholesterol, a fatty acid, a hydrophobic peptide, ergosterol, alkyl chain, di-o-alkyl-rac-glycerol, fullerene, or adamantane. Cell coupling agents can also be long chain fatty acids, for e.g., myristic acid (tetradecanoic acid), palmitic acid (hexadecanoic acid), or other fatty acids of varied lengths. Oligonucleotides may be conjugated to small lipophilic molecules. See Gosse. et. al, J. Phys. Chem. B 2004, 108: 6485-6497, which is entirely incorporated herein by reference. The cell coupling agent may comprise a fusogenic lipid, cholesterol and a PEG lipid. In some instances, the formulation may have a molar ratio 50:10:38.5:1.5-3.0 (cationic lipid:fusogenic lipid:cholesterol:PEG lipid). The PEG lipid may be selected from, but is not limited to PEG-c-DOMG, PEG-DMG. The fusogenic lipid may be DSPC.

In some instances, the cell coupling agent may comprise a nanoparticle with at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG and PEGylated lipids. In another aspect, the lipid may be a cationic lipid such as, but not limited to, DLin-DMA, DLin-D-DMA, DLin-MC3-DMA, DLin-KC2-DMA and DODMA. The lipid to the oligonucleotide ratio may be between 10:1 and 30:10. The PDI of the nanoparticle formulation comprising the modified mRNA may be between 0.03 and 0.15. The zeta potential of the lipid may be from −10 to +10 at a pH of 7.4.

Several methods for the conjugation of lipids with oligonucleotides are known in the art. For example, the oligonucleotide may have two or more hydrophobic anchoring moieties contact a lipid membrane, thereby accomplishing a direct attachment of the oligonucleotide by the moieties at adjacent sites on the same membrane. Synthetic oligonucleotides with a lipidic phosphoramidite may be chemically synthesized that can be passively incorporated into cell membranes as described in Selden et. al, JACS. 2011 28;134(2):765-8, which is entirely incorporated herein by reference. The oligonucleotide-lipid conjugates may be prepared in interaction with micelles such as with 1-octyl-β-d-glucopyranoside micelles or egg phosphatidylcholine vesicles. In some examples, hydrophobic derivatives such as steroid derivatives of oligonucleotides may be attached to the cell surface through non-covalent interactions. For example, palmitoyl derivatives of thiophosphoryl oligonucleotides may be used in complex with low-density lipoproteins to target the cell surface. In some examples, lipid domains may be designed for use as membrane-anchoring moieties with oligonucleotide-derivatives to target the cell surface (Borisenko et. al, Nucleic Acids Research, 2009, which is entirely incorporated herein by reference).

Oligonucleotides/cationic polymer nanoparticles (polyplexes) may be used to deliver the oligonucleotide-conjugate to the surface of the cell. For example, PEG hydrogels loaded with DNA/PEI polyplexes through may be used to deliver oligonucleotides to the cell surface. Hyaluronic acid or fibrin hydrogels loaded with unaggregated polyplexes may also be used to deliver oligonucleotide-complexes to the cell surface.

Oligonucleotides may be chemically modified before conjugation to a cell coupling agent (e.g., lipids), to protect the oligonucleotides from degradation, or to alter the stability and other desirable properties of the oligonucleotides. Chemical modifications may occur at three different sites: (i) at phosphate groups, (ii) on the sugar moiety, and/or (iii) on the entire backbone structure of the oligonucleotide as described herein. In other examples, nucleotide analogues may be modified to improve the amphiphilic properties of the oligonucleotide-complex and subsequently enhance membrane uptake. For example, the oligonucleotide may be modified on the sugar-phosphate background to include thio-phosphoryl and thiophosphoramidate.

The cell coupling agent may be a nanoparticle. The nanoparticle may be metal nanoparticles, carbon-based nanoparticles, ceramic nanoparticle, semin-conductor nanoparticles, polymeric nanoparticles or lipid based nanoparticles. In some examples, the nanoparticle may be a Quantum Dot (QD). QDs may be functionalized for conjugation with oligonucleotides (e.g., universal primer) using several strategies such as discussed in Banerjee et. al, 2016, Ineterface Focus, 6 (6):20160064, which is entirely incorporated herein by reference. For example, the natural affinity of functional groups on chemically modified DNA such as thiol, polyhistidine and phosphorothioate modifications to the inorganic shells of QDs can be used for non-covalent attachment of oligonucleotides to the surface of QDs. For example, QD-DNA conjugation can involve ligand exchange of QDs first with mercaptopropionic acid (MPA) followed by displacement of MPA by thiol-functionalized DNA (DNA-SH) directly. In other examples, polyanionic phosphodiester backbone of DNA can be adsorbed by cationic surface coatings on the QDs via electrostatic interactions. Another variation of this strategy may involve the affinity of polyhistidine tagged DNA directly to the surface of QDs.

Lipid oligonucleotide conjugates (LONs) may be embedded within the amphipathic capping layer on QDs by hydrophobic interactions. See Aime et.al, 2013 Bioconjug. Chem. 24, 1345-1355, which is entirely incorporated herein by reference. In this method of conjugation of DNA, the oligonucleotides may be first conjugated to the amphiphilic lipids and then added in the overall encapsulating formulation to directly display conjugated DNA on QDs. QDs may be surface-functionalized with specific organic or biomolecular ligands for conjugation with oligonucleotides. Commercial bifunctional linkers such as SMCC, SPDP, MBS etc. may be used to covalently to form QD-oligonucleotide conjugates. In another example, amine-functionalized DNA (DNA-NH2) may be coupled to QDs by reaction with 1-ethyl-3-(3-(dimethylamino)-propyl)carbodiimide hydrochloride (EDC) and N-hydroxysuccinimide (NHS). In one example, Ni-NTA-modified QDs may be complexed with polyhistidine-modified DNA. Alternately, QDs can be functionalized with streptavidin first, followed by addition of a biotinylated DNA. QDs conjugated with oligonucleotides may be linked with membrane receptor proteins for directing an oligonucleotide-QD complex to the cell surface. Encapsulated or aqueous QDs may be derivatized to proteins or peptides and other biomolecules using straightforward bioconjugation methods used routinely in the art. QDs may be conjugated to membrane receptor proteins as has been shown previously with NGF receptors, acetylcholine receptor, acetylcholine receptor or GPCRs. Peptides that serve as ligands for membrane-bound receptors have been conjugated to QDs for cell membrane targeting. See Barroso et al., J Histochem Cytochem. 2011 March; 59(3): 237-251, which is entirely incorporated herein by reference.

Cell coupling agents comprising oligonucleotides conjugated to streptavidin-linked QDs may be directed to the cell surface using a biotinylated cell surface protein. For example, cells of interest may carry a cell surface protein genetically encoded to carry the Acceptor Peptide (AP), GLNDIFEAQKIEWHE at the N terminus or C terminus of the protein of interest. The Acceptor Peptide may be biotinylated in the presence of ATP and biotin by recombinantly expressed Biotin ligase (BirA) and excess biotin removed by washing. In other examples, the cell membrane may be engineered with biotin groups by incubating the cells of interest with cholesterol-PEG2k-biotin. QD-oligonucleotide conjugates functionalized with avidin may then be actively recruited to the cell surface.

The cell coupling agent may also be a carbohydrate. Oligonucleotide-carbohydrate conjugates may have monovalent ligands (one carbohydrate group), binary oligo-saccharides (two carbohydrate groups attached via a linkage), trinary oligosaccharides (three carbohydrate groups each attached via a linkage to the scaffold) or more for higher affinity. Oligonucleotide-carbohydrate conjugates (glyco-oligo conjugates) may target the carbohydrate recognition domains (CRD) found on receptors such as the asiologlycoprotein-receptor (ASGP-R). Examples of carbohydrate moieties that are recognized by the carbohydrate recognition domains (CRD) found on the asiologlycoprotein-receptor (ASGP-R) include Galactose and lactose, mannose, sialic acid etc.

In some examples, sugars may be attached to small peptides to mimic multivalent N-linked oligosaccharides that can be recognized by carbohydrate-recognition domains on the cell surface. See Haensler et al., Bioconjugate Chem., 1993, 4, 85, which is entirely incorporated herein by reference. In some cases, cell surface oligosaccharides may be chemically modified to incorporate a functional azido group and the surface sialic azides may be conjugated via covalent linkage with synthetic oligonucleotides modified with phosphine group via Staudinger ligation.

Oligonucleotides-carbohydrate complexes may be targeted to cells via a carrier or high affinity ligand for surface carbohydrate-type receptors that mediate uptake of various ligands. Carrier or high affinity ligands that may be used for glycotargeting of surface carbohydrate-type receptors include glycoproteins or neo glycoproteins, glycopeptides or neoglycopeptides or glycosylated polymers.

Membrane attachment complexes carrying specific cell surface receptor ligands such as carbohydrates may be selectively directed towards cells that have surface receptors recognizing the ligand. For example, glycoproteins having exposed terminal carbohydrate groups such as asialoglyco-protein (galactose-terminal) can be used to target hepatocytes, although other ligands such as polypeptide hormones may also be employed. Such ligands can be formed by chemical or enzymatic desialylation of glycoproteins that possess terminal sialic acid and penultimate galactose residues. Alternatively, asialoglycoprotein ligands can be formed by coupling galactose terminal carbohydrates such as lactose or arabinogalactan to non-galactose bearing proteins by reductive lactosamination. Other types of ligands may be used for targeting the molecular conjugate to cell-surface receptors, such as mannose for macrophages (lymphoma), mannose-6-phosphate glycoproteins for fibroblasts (fibrosarcoma).

In other cases, the cell coupling agent may comprise a protein, or a functional domain or functional fragment thereof that can bind to carbohydrate motifs expressed on the cell surface. For example, the cell coupling agent may be selected from, but not limited to Lectins, L-selectins, E-selectins, P-selectins or a functional domain or functional fragment thereof. Examples of Lectins include but are not limited to Concanavilin A, Griffonia simplicifolia lectin 4, wheat germ agglutinin, Ricin, Galectin-1, Mannose-binding protein, Influenza Virus hemagglutinin, Polyoma virus protein 1, Enterotoxin, Cholera toxin, etc.

Non-covalently linked oligonucleotide-neoglycoprotein complexes may be used for cell surface targeting, for example biotinylated oligonucleotides may be non-covalently linked with mannosylated streptavidin. Oligonucleotides may be non-covalently linked with asialoglycoprotein-polylysine conjugates for more efficient transmembrane uptake. See Bunnel et al., Somatic Cell Molecular Genetics, 1992, 18, 559; Reinis et al., J. Virol. Meth., 1993, 42, 99, each of which is entirely incorporated by reference. For example, oligonucleotides conjugated to synthetic neoglycoproteins with target affinity for lectins such as ASGP-R, containing galactopyranosyl residues at non-reducing terminal positions may be used to target the cell surface.

Carbohydrate-oligonucleotide conjugates may be prepared through the preparation of carbohydrate containing phosphoramidites. Click chemistry may be used to synthesize oligonucleotide glycoconjugates including branched structures. See Pourceau et. al, J. Org. Chem., 2009, 74 (3), pp 1218-22, which is entirely incorporated herein by reference. Glycoconjugates may be delivered to a specific cell type via targeting the asialoglycoprotein receptor, a cell surface lectin found on hepatocytes. See Akinc et. al, Mol Ther. 2010 July; 18(7): 1357-1364, which is entirely incorporated herein by reference. Carbohydrate ligands may be linked to the oligonucleotide or oligonucleoside via linker molecules as described in U.S. Pat. No. 6,660,720, which is entirely incorporated herein by reference.

In some instances, small molecule ligands with high affinity to specific cell surface receptors may be used as cell coupling agents for the design of oligonucleotide conjugates targeted to the cell surface. For example, solid phase DNA synthesis may be used to prepare monovalent or trivalent anisamide-oligonucleotide conjugates, anisamide being a ligand for the sigma receptor. Phosphoramidite version of anisamide, a ligand for the sigma receptor may be converted from N-[2-(2-hydroxyethoxy)ethyl]-4-methoxybenzamide, a derivative of anisamide with a reactive hydroxyl group. See Nakagawa et. al, J Am Chem Soc. 2010 Jul. 7; 132(26):8848-49, which is entirely incorporated herein by reference. In another example, analogs of clozapine and CNO may be reacted with 1,1′-carbonyldiimidazole and conjugated to a single-stranded oigonucleotide having a 5′-aminolinker to produce small-molecule Oligonucleotide conjugates directed to a specific GPCR that responded to clozapine or clozapine-N-oxide. See Alam et al., Bioorg Med Chem. 2013 Oct. 15; 21(20): 6217-6223, which is entirely incorporated herein by reference.

In some cases, the cell coupling agent may be an oligonucleotide binding agent such as a polycation. Suitable polycations are polylysine, polyarginine, polyornithine, and basic proteins such as histones, avidin, protamines and the like. In some examples, complexes comprising of DNA, polycation, and a polysaccharide (e.g Schizophyllan) may be used to induce cellular uptake of the complex by a target cell of interest. In some instances, laterally stabilized complexes of DNA with linear reducible polycations, induced by coating with PHPMA, may be targeted to cell surface receptors for specific transmembrane uptake. See Oupicky et. al, J. Am. Chem. Soc., 2002, 124 (1): 8-9, which is entirely incorporated herein by reference.

Cell coupling agents may be conjugated with oligonucleotides through functional groups introduced into the cell coupling agent, the oligonucleotide or both. In some examples, a disulfide bond may be created between a thiol modified oligonucleotide and active electrophilic S atoms introduced by exposing the N-succinimidyl 3-(2-pyridyldithio) propionate (SPDP). The active electrophilic S bonds may be introduced into the cell coupling agents or the cell surface directly. For example, free thiols on the cell surface can then react with free thiols of an oligonucleotide comprising another disulfide bond (e.g., via thiol-disulfide exchange) such that the oligonucleotide-conjugate can be linked to the cells (e.g., via a generated disulfide bond). In some cases, free thiols of the cells may react with any other suitable group. For example, free thiols on the cell surface may react with an oligonucleotide comprising an acrydite moiety. The free thiol groups of the cell surface can react with the acrydite via Michael addition chemistry, such that the oligonucleotides comprising the acrydite may be linked to the cell surface. In some cases, uncontrolled reactions can be prevented by inclusion of a thiol capping agent such as ethylmalieamide or iodoacetate.

Conjugation of oligonucleotides with cell coupling agents may involve chemical modifications of oligonucleotides. Chemically modified nucleotides may contain active groups to which a cell coupling agent can then be linked. In some cases, one or more nucleotides of the oligonucleotide can be modified to improve the oligonucleotide's cellular uptake, cell-permeability, or for improved conjugation with the cell coupling agent. Several methods have been developed for syntheses of oligonucleotide conjugates using chemically modified oligonucleotides, including, for example, incorporation of modified phosphoramidites, step-wise (in-line) solid-phase synthesis, on-support fragment coupling, and solution-phase coupling.

The oligonucleotides disclosed herein can comprise one or more chemical modifications at various locations, including at a sugar moiety, a phosphodiester linkage, and/or a base. The oligonucleotide can comprise natural, chemically modified, biochemically modified, non-natural, synthetic or derivatized nucleotide bases. For example, the oligonucleotides can comprise a backbone that comprises phosphoramide, phosphorothioate, phosphorodithioate, boranophosphate linkage, O-methylphosphoramidite linkages, and/or peptide nucleic acids. The oligonucleotides can comprise a 2′fluoro-arabino nucleic acid, tricycle-DNA (tc-DNA), peptide nucleic acid, cyclohexene nucleic acid (CeNA), locked nucleic acid (LNA), a locked nucleic acid (LNA) nucleotide comprising a methylene bridge between the 2′ and 4′ carbons of the ribose ring, bridged nucleic acids (BNA), ethylene-bridged nucleic acid (ENA), a phosphodiamidate morpholino, or a combination thereof.

The oligonucleotides can comprise one or more non-naturally occurring nucleotides or nucleotide analogs, e.g., a nucleotide with phosphorothioate linkage, boranophosphate linkage, a locked nucleic acid (LNA) nucleotide comprising a methylene bridge between the 2′ and 4′ carbons of the ribose ring, or bridged nucleic acids (BNA). The non-naturally occurring nucleotides or nucleotide analogs can be 2′-0-methyl analogs, 2′-deoxy analogs, 2-thiouridine analogs, N6-methyladenosine analogs, or 2′-fluoro analogs.

The oligonucleotides can comprise one or more modified bases. The one or more modified bases can be 2-aminopurine, 5-bromo-uridine, pseudouridine (Ψ), N\hat{ }methylpseudouridine (mel P), 5-methoxyuridine(5moU), inosine, or 7-methylguanosine.

The oligonucleotides can comprise a sugar moiety. The sugar moieties can be natural, unmodified sugar, e.g., monosaccharide (e.g., pentose, e.g., ribose, deoxyribose), modified sugars, or sugar analogs. In some cases, the sugar moiety can have or more hydroxyl groups replaced with a halogen, a heteroatom, an aliphatic group, or the one or more hydroxyl groups can be functionalized as an ether, an amine, a thiol, or the like.

The oligonucleotides can comprise one or more modifications at a 2′ position of a ribose. The 2′ moiety can be H, OR, R, halo, SH, SR, H2, HR, R2 or ON, wherein R is Ci-C6 alkyl, alkenyl or alkynyl and halo is F, CI, Br or I. Examples of sugar modifications include 2′-deoxy-2′-fluoro-oligoribonucleotide (2′-fluoro-2′-deoxycytidine-5′-triphosphate, 2′-fluoro-2′-deoxyuridine-5′-triphosphate), 2′-deoxy-2′-deamine oligoribonucleotide (2′-amino-2′-deoxycytidine-5′-triphosphate, 2′-amino-2′-deoxyuridine-5′-triphosphate), 2′-0-alkyl oligoribonucleotide, 2′-deoxy-2′-C-alkyl oligoribonucleotide (2′-O-methylcytidine-5′-triphosphate, 2′-methyluridine-5′-triphosphate), 2′-C-alkyl oligoribonucleotide, and isomers thereof (2′-aracytidine-5′-triphosphate, 2′-arauridine-5′-triphosphate), azidotriphosphate (2′-azido-2′-deoxycytidine-5′-triphosphate, 2′-azido-2′-deoxyuridine-5′-triphosphate), and combinations thereof. The sugar-modified ribonucleotides can have the 2′ OH group replaced by a H, alkoxy (or OR), R or alkyl, halogen, SH, SR, amino (such as NH2, NHR, NR2), or CN group, wherein R is lower alkyl, alkenyl, or alkynyl.

The oligonucleotides may comprise one or more nucleobase-modified ribonucleotides. The one or more modified ribonucleotides may contain a non-naturally occurring base (instead of a naturally occurring base), such as uridines or cytidines modified at the 5′-position, e.g., 5′ (2-amino)propyl uridine or 5′-bromo uridine; adenosines and guanosines modified at the 8-position, e.g., 8-bromo guanosine; deaza nucleotides, e.g., 7-deaza-adenosine; and N-alkylated nucleotides, e.g., N6-methyl adenosine.

The nucleobase-modified nucleotides may be m5C (5-methylcytidine), m5U (5-methyluridine), m6A (N6-methyladenosine), s2U (2-thiouridine), Um (2′-0-methyluridine), m1A (1-methyl adenosine), m2A (2-methyladenosine), Am (2-1-O-methyladenosine), ms2m6A (2-methylthio-N6-methyladenosine), i6A (N6-isopentenyl adenosine), ms2i6A (2-methylthio-N6isopentenyladenosine), io6A (N6-(cis-hydroxyisopentenyl) adenosine), ms2io6A (2-methylthio-N6-(cis-hydroxyisopentenyl)adenosine), g6A (N6-glycinylcarbamoyladenosine), t6A (N6-threonyl carbamoyladenosine), ms2t6A (2-methylthio-N6-threonyl carbamoyladenosine), m6t6A (N6-methyl-N6-threonylcarbamoyladenosine), hn6A (N6.-hydroxynorvalylcarbamoyl adenosine), ms2hn6A (2-methylthio-N6-hydroxynorvalyl carbamoyladenosine), Ar(p) (2′-0-ribosyladenosine(phosphate)), I (inosine), mi 1 (1-methylinosine), m′lm (1,2′-0-dimethylinosine), m3C (3-methylcytidine), Cm (2T-0-methylcytidine), s2C (2-thiocytidine), ac4C (N4-acetylcytidine), f5C (5-fonnylcytidine), m5Cm (5,2-O-dimethylcytidine), ac4Cm (N4acetyl2TOmethylcytidine), k2C (lysidine), m1G (1-methylguanosine), m2G (N2-methylguanosine), m7G (7-methylguanosine), Gm (2′-0-methylguanosine), m22G (N2,N2-dimethylguanosine), m2Gm (N2,2′-0-dimethylguanosine), m22Gm (N2,N2,2′-0-trimethylguanosine), Gr(p) (2′-0-ribosylguanosine(phosphate)), yW (wybutosine), o2yW (peroxywybutosine), OHyW (hydroxywybutosine), OHyW* (undermodified hydroxywybutosine), imG (wyosine), mimG (methylguanosine), Q (queuosine), oQ (epoxyqueuosine), galQ (galtactosyl-queuosine), manQ (mannosyl-queuosine), preQo (7-cyano-7-deazaguanosine), preQi (7-aminomethyl-7-deazaguanosine), G (archaeosine), D (dihydrouridine), m5Um (5,2′-0-dimethyluridine), s4U (4-thiouridine), m5s2U (5-methyl-2-thiouridine), s2Um (2-thio-2′-0-methyluridine), acp3U (3-(3-amino-3- carboxypropyl)uridine), ho5U (5-hydroxyuridine), mo5U (5-methoxyuridine), cmo5U (uridine 5-oxyacetic acid), mcmo5U (uridine 5-oxyacetic acid methyl ester), chm5U (5-(carboxyhydroxymethyl)uridine)), mchm5U (5-(carboxyhydroxymethyl)uridine methyl ester), mcm5U (5-methoxycarbonyl methyluridine), mcm5Um (S-methoxycarbonylmethyl-2-O-methyluridine), mcm5s2U (5-methoxycarbonylmethyl-2-thiouridine), nm5s2U (5-aminomethyl-2-thiouridine), mnm5U (5-methylaminomethyluridine), mnm5s2U (5-methylaminomethyl-2-thiouridine), mnm5se2U (5-methylaminomethyl-2-selenouridine), ncm5U (5-carbamoylmethyl uridine), ncm5Um (5-carbamoylmethyl-2′-0-methyluridine), cmnm5U (5-carboxymethylaminomethyluridine), cnmm5Um (5-carboxymethylaminomethyl-2-L-Omethyluridine), cmnm5s2U (5-carboxymethylaminomethyl-2-thiouridine), m62A (N6,N6-dimethyladenosine), Tm (2′-0-methylinosine), m4C (N4-methylcytidine), m4Cm (N4,2-0-dimethylcytidine), hm5C (5-hydroxymethylcytidine), m3U (3-methyluridine), cm5U (5-carboxymethyluridine), m6Am (N6,T-0-dimethyladenosine), rn62Am (N6,N6,0-2-trimethyladenosine), m2′7G (N2,7-dimethylguanosine), m2′2′7G (N2,N2,7-trimethylguanosine), m3Um (3,2T-0-dimethyluridine), m5D (5-methyldihydrouridine), f5Cm (5-formyl-2′-0-methylcytidine), m1Gm (1,2′-0-dimethylguanosine), m′Am (1,2-0-dimethyl adenosine)irinomethyluridine), tm5s2U (S-taurinomethyl-2-thiouridine)), imG-14 (4-demethyl guanosine), imG2 (isoguanosine), or ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(Ci-C6)-alkyluracil, 5-methyluracil, 5-(C2-C6)-alkenyluracil, 5-(C2-C6)-alkynyluracil, 5-(hydroxymethyl)uracil, 5-chlorouracil, 5-fluorouracil, 5-bromouracil, 5-hydroxy cytosine, 5-(Ci-C6)-alkylcytosine, 5-methylcytosine, 5-(C2-C6)-alkenylcytosine, 5-(C2-C6)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, and combinations thereof.

The nucleobase-modified nucleotides can be Aminopurine, 2,6-Diaminopurne (2-Amino-dA), 5-Bromo dU, deoxyuridine, Inverted dT, Inverted Dideoxy-T, dideoxy-C, 5-Methyl dC, Super (T), Super (G), 5-Nitroindole, 2′-O-Methyl RNA Bases, Hydroxymetyl dC, Iso dG, Iso dC, Fluoro C, Fluoro U, Fluoro A, Fluoro G, 2-MethoxyEthoxy MeC, 2-MethoxyEthoxy G, or 2-MethoxyEthoxyT.

In some cases, the 3′ and 5′ termini of the oligonucleotides can be substantially protected from nucleases, e.g., by modifying the 3′ or 5′ linkages. For example, the oligonucleotides can be made resistant by the inclusion of one or more blocking groups. The one or more end-blocking groups can be a cap structure (e.g., a 7-methylguanosine cap), inverted nucleomonomer, e.g., with 3′-3′ or 5′-5′ end inversions, methylphosphonate, phosphoramidite, non-nucleotide groups (e.g., non-nucleotide linkers, amino linkers, conjugates) and the like. The 3′ terminal nucleomonomer can comprise a modified sugar moiety. For example, the 3′-hydroxyl can be esterified to a nucleotide through a 3′→3′ internucleotide linkage. For example, the alkyloxy radical can be methoxy, ethoxy, or isopropoxy. Optionally, the 3′→3′ linked nucleotide at the 3′ terminus can be linked by a substitute linkage. To reduce nuclease degradation, the 5′ most 3′→5′ linkage can be a modified linkage, e.g., a phosphorothioate or a P-alkyloxyphosphotriester linkage.

The oligonucleotide may be linked with the cell coupling agent by a linkage. In some cases, a chemical moiety linked to an oligonucleotide can comprise a labile bond, such as chemically, thermally, or photo-sensitive bond e.g., disulfide bond, UV sensitive bond, or the like. Acrydite moieties or other moieties comprising a labile bond may be incorporated into a cell coupling agent or the cell surface. The labile bond may be, for example, useful in reversibly linking (e.g., covalently linking) species (e.g., barcodes, primers, etc.) to a cell. In some cases, a thermally labile bond may include a nucleic acid hybridization based attachment, e.g., where an oligonucleotide is hybridized to a complementary sequence that is attached to the cell, such that thermal melting of the hybrid releases the oligonucleotide, e.g., a barcode containing sequence, from the cell or microcapsule.

The linkage may comprise photocleavable bonds sensitive to light radiations. Oligonucleotides can be synthesized with the incorporation of modified nucleotides containing a photolabile group, that is susceptible to cleavage by specific wavelengths of light. For instance, the appropriate conditions can be exposure to UV light. A photolabile group can be introduced into the oligonucleotide by phosphoramidite chemistry. Selective reaction of PC-aminotag phosphoramidites with the free 5′-OH group of a growing oligonucleotide chain, followed by cleavage from the support and deprotection, can result in the introduction of a phosphodiester group linked to a primary aliphatic amino group through a photocleavable linker. This amino group may then be used to introduce a variety of photocleavable markers through a postsynthetic modification reaction with amine reactive reagents.

In addition to thermally cleavable bonds, disulfide bonds and UV sensitive bonds, other non-limiting examples of labile bonds that may be coupled to a cells include an ester linkage (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)).

In some instances, a cleavable linker may be introduced into the oligonucleotide using a modified internucleoside linkage. The modified internucleoside linkage can be an internucleotide linkage that has a phosphorus atom or those that do not have a phosphorus atom. Internucleoside linkages containing a phosphorus atom therein include, for example, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, P-ethyoxyphosphodiester, P-ethoxyphosphodiester, P-alkyloxyphosphotriester, methylphosphonate, thionophosphoramidates, thionoalkylphosphonates, thionoalkylphosphotriesters, selenophosphates and boranophosphates, and nonphosphorus containing linkages, e.g., acetals and amides, such as are known in the art, having normal 3′-5′ linkages, 2′-5′ linked analogs of these, and those having inverted polarity wherein one or more internucleotide linkages is a 3′ to 3′, 5′ to 5′ or 2′ to 2′ linkage. Polynucleotides having inverted polarity can comprise a single 3′ to 3′ linkage at the 3′-most internucleotide linkage i.e. a single inverted nucleoside residue which may be abasic (the nucleobase is missing or has a hydroxyl group in place thereof).

Non-phosphorus containing internucleoside linkages include short chain alkyl, cycloalkyl, mixed heteroatom alkyl, mixed heteroatom cycloalkyl, one or more short chain heteroatomic and one or more short chain heterocyclic. These internucleoside linkages include but are not limited to siloxane, sulfide, sulfoxide, sulfone, acetyl, formacetyl, thioformacetyl, methylene formacetyl, thioformacetyl, alkeneyl, sulfamate; methyleneimino, methylenehydrazino, sulfonate, sulfonamide, amide and others having mixed N, O, S and CH2 component parts. Other modified internucleoside linkages that do not contain a phosphorus atom therein include, —CH₂—NH—O—CH₂—, —CH₂—N(CH₃)—O—CH₂— (known as a methylene (methylimino)backbone), —CH₂—O—N(CH₃)—CH₂—, —CH₂—N(CH₃)—N(CH₃)—CH₂— and —O—N(CH₃)—CH₂—CH₂—.

The cleavable linker can be non-nucleotide in nature. A “non-nucleotide” can refer to any group or compound that can be incorporated into a polynucleotide chain in the place of one or more nucleotide units, including either sugar and/or phosphate substitutions. The group or compound can be abasic in that it does not contain a commonly recognized nucleotide base, such as adenosine, guanine, cytosine, uracil or thymine, for example at the C1 position of the sugar.

Non-nucleotidic linkers can be e.g., abasic residues (dSpacer), oligoethyleneglycol, such as triethyleneglycol (spacer 9) or hexaethylenegylcol (spacer 18), or alkane-diol, such as butanediol. The spacer units can be preferably linked by phosphodiester or phosphorothioate bonds. The linker units may appear just once in the molecule or may be incorporated several times, e.g., via phosphodiester, phosphorothioate, methylphosphonate, or amide linkages. Further preferred linkers are alkylamino linkers, such as C3, C6, C12 aminolinkers, and also alkylthiol linkers, such as C3 or C6 thiol linkers. In some examples, heterobifunctional and homobifunctional linking moieties may be used to conjugate peptides and proteins to nucleotides. Examples include 5′-Amino-Modifier C6 and 3′-Amino-Modifier C6 reagents.

The oligonucleotide may also be conjugated with a variety of molecules such as steroids, reporter molecules, reporter enzymes, vitamins, non-aromatic lipophilic molecules, chelators, porphyrins, intercalators, peptides and proteins through an intermediate linking group, such as ω-aminoalkoxy and ω-aminoalkylamino groups, to enable better transmembrane uptake. Conjugation has been reported at the 3′-, 5′-, 2′-, internucleotide linkage and nucleobase positions of oligonucleotides. See, e.g., Manoharan et al. PCT Application WO 93/07883, which is entirely incorporated herein by reference.

One or more cell coupling agents that target a cell can be used to generate a cell comprising a plurality of cell coupling agents. In some instances, the plurality of the cell coupling agents are the same. In some instances, the plurality of the cell coupling agents are the different. For example, a cell may comprise, couples to the surface of the cell, a first cell coupling agent (e.g. comprising an antibody) and a second cell coupling agent (e.g. comprising a lipophilic molecule). As another example, a cell may comprise, couples to the surface of the cell, a first type of cell coupling agent comprising a first lipophilic molecule and a second cell coupling agent comprising a second lipophilic molecule, wherein the first and second lipophilic molecules are different. In some instances, 1, 2, 3, 4, 5, 6, 7, 8, 9, 10, or more different cell coupling agents are utilized.

### Split Pool for Barcode Extension

In some cases, the nucleic acid sequence of an oligonucleotide tag can be generated on the surface of the cell using a split pool method, such that in each cycle of split pooling, a portion (e.g., partial barcode sequence) of the oligonucleotide tag is generated and/or appended. The split-pool cycles may be repeated or iterated two times or more, for any number of suitable cycles for highly multiplexed generation of barcode sequences associated with the surface of a cell.

The nucleotide or oligonucleotide sequence on the surface of the cell is a molecular barcode. The molecular barcode may be a composite barcode sequence composed of at least 2 individual constituent barcodes (partial barcodes or barcode subsequences).

A method for preparing a plurality of surface-barcoded cells wherein the barcode comprises a unique nucleic acid sequence can comprise: iterative extension of an oligonucleotide on the surface of the plurality of cells (e.g., an oligonucleotide coupled to a cell coupling agent as described elsewhere herein) in a pool-and-split process, such that in each cycle, the cells are split into a plurality of partitions where, in each partition, the cell surface is subjected to a nucleic acid reaction (such as a ligation or extension reaction) comprising the extension of the barcode with an additional oligonucleotide sequence; and repeating the pool-and-split process for any number of cycles. For example, there may be at least 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000 or more cycles.

An individual cell surface may be modified (e.g., chemically, enzymatically, or via a cell coupling agent as described elsewhere herein) to be coupled to any number of individual nucleic acid molecules, for example, from one to tens to hundreds of thousands or even millions of individual nucleic acid molecules. The individual nucleic acid molecules can comprise both common sequence segments or relatively common sequence segments and variable or unique sequence segments between different individual nucleic acid molecules coupled to the same cell. For example, individual oligonucleotide tags attached to a given cell may all comprise a common barcode sequence (e.g., constituent barcode sequence comprised of split-pool generated partial barcode sequences), but also comprise a variable unique molecule index (UMI).

Disclosed herein, in some embodiments, are methods for simultaneously preparing a plurality of indexed cells comprising: forming a mixture comprising a plurality of cells wherein the cell surface carries a cell coupling agent (e.g., as described herein) linked to a nucleic acid molecule comprising a universal adapter sequence; separating the cells into partitions; extending the sequence of the nucleic acid molecule on the surface of the cells by appending a first partial barcode sequence to the nucleic acid molecule (e.g., via ligation to the universal adapter sequence); pooling the cells from the partitions into a single common pool; and repeating the partitioning and partial barcode addition multiple times to produce, combinatorially through an iterative split pool approach, a plurality of oligonucleotide tags comprising a composite barcode sequence linked or attached to the respective cell surfaces of the cells (indexed cells).

Appending or attaching a first barcode nucleic acid barcode molecule, or sequence thereof, can be achieved by 5′ or 3′ addition to a second barcode nucleic acid molecule. In some instances, an appending or attaching is achieved by ligation, hybridization, or any combination thereof. For example, a first barcode molecule and a second barcode may be directly coupled. In another example, a splint oligonucleotide molecule may be used to attach a first barcode molecule and a second barcode molecule. The splint oligonucleotide molecule may comprise a double-stranded region and one or more single-stranded regions. For example, the splint oligonucleotide molecule may comprise a central portion that is a double-stranded region and adjacent regions on either side that are single-stranded regions. The single-stranded regions of the molecule may be on the same or different strands. The single-stranded region on one or both ends of the molecule may comprise 1, 2, 3, 4, 5, 6, or more nucleotides. This single-stranded region may be referred to as comprising an “overhang” sequence. A first overhang sequence may comprise a sequence that is complementary to a portion of a first barcode molecule and a second overhang sequence may comprise a sequence that is complementary to a portion of a second barcode molecule. The complementary sequence may comprise any useful length and base composition. Similarly, the double-stranded region may comprise any useful length and composition. The first barcode molecule may hybridize to the first overhang sequence and ligated. The second barcode molecule may hybridize to the second overhang sequence and ligated. In some cases, the splint oligonucleotide molecule may comprise a barcode sequence for combinatorial assembly, such that a nth barcode molecule is itself a splint oligonucleotide molecule. An assembled, double-stranded molecule comprising a composite barcode sequence may be converted to a single strand by denaturing to remove a strand and retaining a single-stranded nucleic acid barcode molecule attached to the cell surface. Alternatively, denaturation may be performed after the double-stranded nucleic acid barcode molecule is decoupled (e.g., released) from the surface. Methods for assembling barcode sequences and other functional sequences while attached to a surface, including via a splint oligonucleotide, are described further in US2020/0063191, which is entirely incorporated herein by reference. A splint oligonucleotide can comprises one or more functional sequences such as an adapter sequence, a primer or primer binding sequence (e.g., a sequencing primer or sequencing primer binding site), a unique molecular index (UMI), a sequence configured to attach to the flow cell of a sequencer (e.g., an Illumina P5, P7, or partial sequence thereof), a capture sequence configured to hybridize to a specific sequence or molecule (e.g., poly-T sequence configured to attach to a poly-A containing molecule, such as an mRNA), etc. Additionally, the one or more functional sequences can be added after any round of split-pool, during the assembly of a composite barcode molecule. In some instances, the one or more functional sequences are added to at the final round of split-pool.

In some instances, ligation (e.g. enzymatic or chemical) is used to couple a first barcode molecule and a second barcode molecule. In some instances, a hybridization is used to couple a first barcode molecule and a second barcode molecule. In certain instances, splint oligonucleotides are utilized to couple a first and a second barcode molecule.

Furthermore, provided herein in some embodiments are methods for detecting the occurrence of a particular cellular surface constituent. For example, a oligonucleotides tag comprising a composite barcode sequence as described elsewhere herein is linked to a labeling ligand such as a cell coupling agent (e.g., antibody), wherein the presence of the composite barcode sequence indicates the presence of the cellular constituent that the labeling ligand specifically binds. In some cases, the composite barcode sequence can be linked to an antibody for a specific cellular constituent. If the cellular constituent is present in a sample, the antibody will bind and the barcode sequence can be detected. If the cellular constituent is not present in a sample, the antibody will not bind and the barcode will not be detected above background.

In some instances, a composite barcode sequence may range from 8 to 1000 nucleotides in length. In some instances, the composite barcode sequence can include from about 8 to about 20, 30, 40, 50, 60, 70, 80, 90, 100 or more nucleotides. In some cases, the length of a composite barcode sequence may be about 8, 9, 10, 11, 12, 13, 14, 15, 16, 17, 18, 19, 20 nucleotides or longer. In some cases, the length of a composite barcode sequence may be at least about 8, 9, 10, 11, 12, 13, 14, 15, 16, 17, 18, 19, 20 nucleotides or longer. In some cases, the length of a composite barcode sequence may be at most about 8, 9, 10, 11, 12, 13, 14, 15, 16, 17, 18, 19, or 20 nucleotides. These nucleotides of a composite barcode sequence may be completely contiguous, i.e., in a single stretch of adjacent nucleotides, or they may be separated (i.e., non-contiguous) into two or more subsequences (partial barcode sequences) that are separated by 1 or more nucleotides. In some cases, barcode subsequences are separated by about 2 to about 16 nucleotides. In some cases, barcode subsequences are separated by about 2, 3, 4, 5, 6, 7, 8, 9, 10, 11, 12, 13, 14, 15, 16, 17, 18, 19, 20 nucleotides or more. In some cases, barcode subsequences are separated by at least about 2, 3, 4, 5, 6, 7, 8, 9, 10, 11, 12, 13, 14, 15, 16, 17, 18, 19, 2 nucleotides or more. In some cases, barcode subsequences are separated by at most about 2, 3, 4, 5, 6, 7, 8, 9, 10, 11, 12, 13, 14, 15, 16, 17, 18, 19, 20 nucleotides or more. In some cases, a barcode subsequence may be about 4, 5, 6, 7, 8, 9, 10, 11, 12, 13, 14, 15, 16 nucleotides in length or longer. In some cases, a barcode subsequence may be at least about 4, 5, 6, 7, 8, 9, 10, 11, 12, 13, 14, 15, 16 nucleotides in length or longer. In some cases, the barcode subsequence may be at most about 4, 5, 6, 7, 8, 9, 10, 11, 12, 13, 14, 15, 16 nucleotides in length or shorter.

Accordingly, in some cases, one or more separate “pools” of barcode elements in each partition can then be joined together to produce the final barcode, e.g., using a split-and-pool approach. For example, a first pool may contain x1 elements and a second pool may contain x2 elements; forming a barcode containing an element from the first pool and an element from the second pool may yield, e.g., x1*x2 possible barcodes that could be used, where x1 and x2 may or may not be equal. This process may be repeated any number of times; for example, the barcode may include elements from a first pool, a second pool, and a third pool (e.g., producing x1*x2*x3 possible barcodes), or from a first pool, a second pool, a third pool, and a fourth pool (e.g., producing x1*x2*x3*x4 possible barcodes), etc. There may also be 5, 6, 7, 8, or any other suitable number of pools. Accordingly, even a relatively small number of barcode elements may be used to produce a much larger number of distinguishable barcodes. By way of example, 3 rounds of split-pool with 96 partitions will yield 96\hat{ }3 different barcodes; 4 rounds of split-pool with 96 partitions will yield 96\hat{ }4 different barcodes; 3 rounds of split-pool with 384 partitions will yield 384^3 different barcodes

During a split pooling workflow or any step therein, a partition can comprise a single cell or a plurality of cells. In some instances, a partition comprises a single cell. In some instance, a partition comprises a plurality of cells. Cells may be partitioned such that at least one cell (or cell bead) is present in each partition of a plurality of partitions. Cells may be partitioned such that at least 1; 2; 3; 4; 5; 10; 20; 50; 100; 500; 1,000; 5,000; 10,000; 100,000; 1,000,000; or more cells are present in a single partition. Cells may be partitioned such that at most 1,000,000; 100,000; 10,000; 5,000; 1,000; 500; 100; 50; 20; 10; 5; 4; 3; 2; or 1 cell is present in a single partition. Cells may be partitioned in a random configuration.

The partitions may be any of a variety of different types of partitions, e.g., wells, microwells, tubes, vials, microcapsules, droplets (e.g., aqueous droplets) within an emulsion. During each partitioning cycle, a plurality of cells may be partitioned into a plurality of partitions. In some instances, the plurality of partitions may comprise partitions each having a single cell encapsulated therein. In some instances, the plurality of partitions may comprise partitions each having no cell encapsulated therein. In some instances, the plurality of partitions may comprise partitions each having multiple cells encapsulated therein, where the cells are overloaded. In such cases, beneficially, high throughput may be achieved.

A partition may comprise one or more unique identifiers, such as barcodes. For example, such partition-specific barcodes may be the individual barcode subsequences that, when combined with other subsequences of other partitions, eventually form the composite barcode sequence. Barcodes may be previously, subsequently or concurrently delivered to the partitions that hold the compartmentalized or partitioned cell. For example, barcodes may be delivered into wells previous to, subsequent to, or concurrently with partitioning of cells into the well. In another example, barcodes may be injected into droplets previous to, subsequent to, or concurrently with droplet generation. The delivery of the barcodes to a particular partition allows for the later attribution of the characteristics of the individual cells to the particular partition. Barcodes may be delivered, for example on a nucleic acid molecule (e.g., an oligonucleotide), to a partition via any suitable mechanism. Barcoded nucleic acid molecules can be delivered to a partition via a microcapsule. A microcapsule, in some instances, can comprise a bead (e.g., gel bead). Beads are described in further detail herein.In some cases, barcoded nucleic acid molecules can be initially associated with the microcapsule and then released from the microcapsule for attachment with the cell surface. Release of the barcoded nucleic acid molecules can be passive (e.g., by diffusion out of the microcapsule). In addition or alternatively, release from the microcapsule can be upon application of a stimulus which allows the barcoded nucleic acid nucleic acid molecules to dissociate or to be released from the microcapsule. Such stimulus may disrupt the microcapsule, an interaction that couples the barcoded nucleic acid molecules to or within the microcapsule, or both. Such stimulus can include, for example, a thermal stimulus, photo-stimulus, chemical stimulus (e.g., change in pH or use of a reducing agent(s)), a mechanical stimulus, a radiation stimulus; a biological stimulus (e.g., enzyme), or any combination thereof.

The method may further comprise generating a composite barcode sequence for each cell by a split pool ligation method, wherein the universal handle linked to the cell surface comprises a ligation handle sequence configured to produce a DNA overhang capable of hybridization to a complementary overhang on a first barcode nucleotide sequence, wherein the first barcode nucleotide sequence further comprises a second overhang complementary to a subsequent barcode sequence (which may or may not be a final barcode sequence), wherein the subsequent barcode sequence comprises overhangs complementary to the first barcode sequence and to the next subsequent barcode sequence (which may or may not be a final barcode sequence), wherein an nth subsequent barcode sequence comprises overhangs complementary to the preceding barcode sequence and to the n+1th subsequent barcode sequence, and wherein the final barcode sequence has an overhang complementary to the preceding barcode sequence. In some instances, an nth barcode sequence is selected from a plurality of unique sequences comprising compatible DNA overhangs and 6 to 30 base pairs of unique sequence.

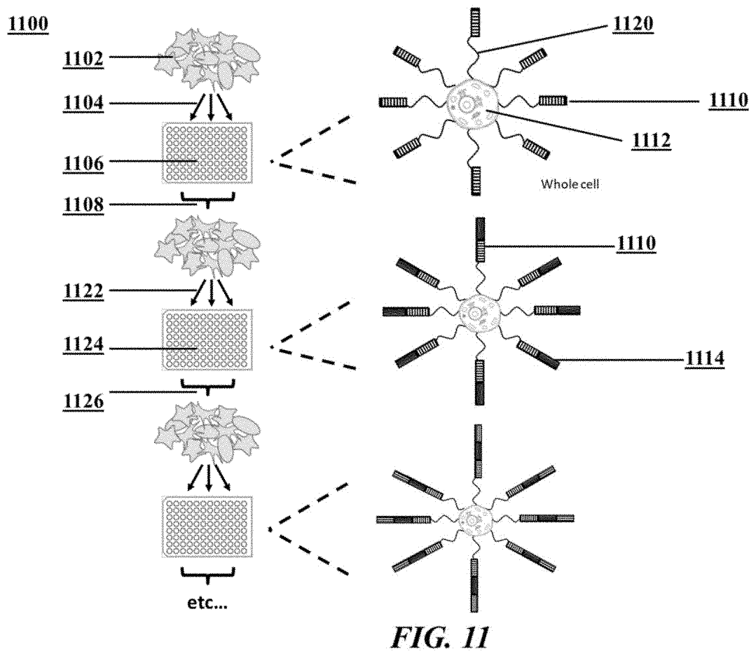

Unique composite barcode sequences may be generated by a split pool method. FIG. 11 shows an exemplary split-pool workflow 1100 for generation of surface-barcoded (indexed) cells. A plurality of cells is surface functionalized using a cell coupling agent (as described elsewhere herein) such that at least some cells each comprise a cell surface-associated nucleic acid molecule comprising a universal adapter sequence (surface-functionalized cells 1102). In some instances, cells are contacted with a coupling agent linked (e.g., covalently) to the nucleic acid molecule comprising the universal adapter sequence 1120. In other instances, cells are first contacted with a coupling agent that associates the coupling agent with the cell surface followed by attachment (e.g., chemical conjugation) of the nucleic acid molecule comprising the universal adapter to the coupling agent thereby generating surface-functionalized cells 1102. Surface-functionalized cells 1102 are then partitioned 1104 (i.e., split) into separate partitions 1106 (e.g., wells of a microwell array), each containing a unique first partial barcode sequence 1110; attaching (e.g., ligating) the first partial barcode sequence 1110 to the universal adapter sequence; pooling 1108 the discrete partitions containing cells (e.g. a plurality of cell 1112); optionally, splitting 1122 the pool of cells into separate partitions 1124 each containing a unique second partial barcode sequence 1114; attaching (e.g., ligating) the second partial barcode sequence to the first partial barcode sequence 1110; and pooling 1126 the cells. The method may comprise optionally repeating the split-pool steps a sufficient number of times to generate the desired barcode diversity. For example, the method may further comprise splitting the pool of cells comprising the first and second partial barcode sequences into partitions containing a third partial barcode sequence; and attaching (e.g., ligating or hybridizing) the third partial barcode sequence to the second partial barcode sequence. This split-pool process can be repeated, whereby after the iterative rounds of split-pool partial barcode addition, each cell surface comprises a unique composite barcode sequence comprised of the partial barcode sequences. The addition of additional barcode sequences and/or attaching barcode sequence (e.g. partial barcode sequences) can be achieved by the methods disclosed herein and methods know in the art. For example, attaching a first barcode sequence (e.g. a partial barcode sequence) can be achieved by ligation, hybridization, or a combination of thereof. In some embodiments, attaching barcode sequences (e.g. partial barcode sequences) comprises hybridizing a first and a second nucleic acid sequence, wherein the first and the nucleic acid sequences are complementary to one another. In some embodiments, attaching barcode sequences (e.g. partial barcode sequences) comprises ligating a first and a second nucleic acid barcode molecule, wherein ligation can be achieved enzymatically or chemically (e.g., chemical ligation, such as using a click chemistry reaction or enzymatic ligation such as using a ligase).

The composite barcode sequence may have at least 2 constituent barcode sequences up to 10000 contiguous barcode sequences or more. In some instances, the number of barcodes may be selected such that the probability of having identical composite barcode sequences on any two single cells approaches zero.

In some instances, the ligation handle sequence may comprise a restriction site for producing an overhang complementary to a first barcode sequence overhang, and the method may comprise digestion with a restriction enzyme. The ligation handle may comprise a sequence complementary with a ligation primer sequence. In some instances, the overhang may be generated by hybridization of the ligation primer to the ligation handle. In some instances, the ligation handle may comprise a double stranded DNA portion that already includes the overhang needed for barcode ligation.

Any suitable number of molecular tag molecules (e.g., primer, barcoded oligonucleotide) can be associated with a cell such that, upon release from the cell, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing nucleic acid molecule (e.g., oligonucleotide) bearing cells.

Cells may contain one or more attached composite barcode molecules comprising barcode sequences. The barcode sequences attached to a single cell may be identical or comprise identical sequences in part. In some cases, the barcode sequences attached to a single cell may be different or comprise different (e.g., unique) sequences in part. In some cases, a cell may be attached to about 1, 5, 10, 50, 100, 500, 1000, 5000, 10000, 20000, 50000, 100000, 500000, 1000000, 5000000, 10000000, 50000000, 100000000, 500000000, 1000000000, 5000000000, 10000000000, 50000000000, or 100000000000 composite barcode molecules. In some cases, a cell may be attached to at least about 1, 5, 10, 50, 100, 500, 1000, 5000, 10000, 20000, 50000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, 2000000000, 3000000000, 4000000000, 5000000000, 6000000000, 7000000000, 8000000000, 9000000000, 10000000000, 20000000000, 30000000000, 40000000000, 50000000000, 60000000000, 70000000000, 80000000000, 90000000000, 100000000000 or more composite barcode molecules. In some cases, a cell may be attached to less than about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 500000, 1000000, 5000000, 10000000, 50000000, 1000000000, 5000000000, 10000000000, 50000000000, or 100000000000 identical and/or different composite barcode molecules.

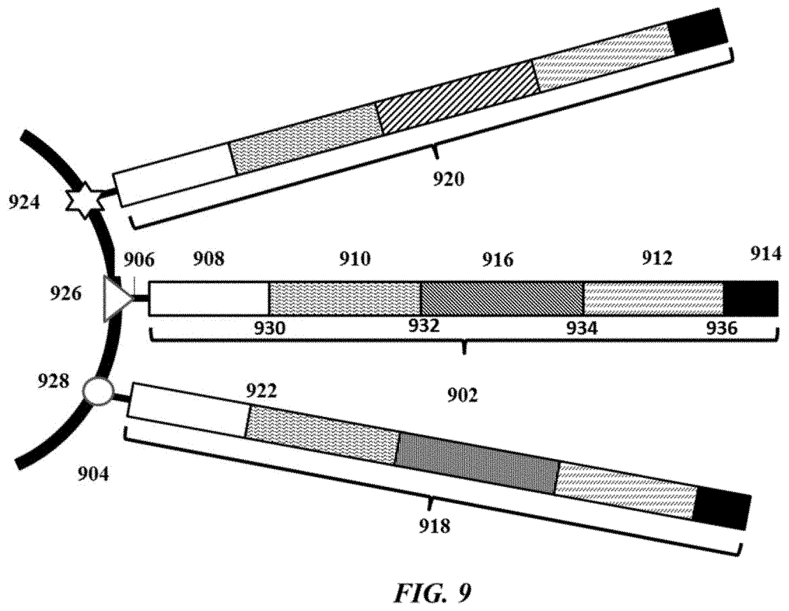

FIG. 9 illustrates an example of a barcode carrying cell. A nucleic acid molecule 902, comprising a universal handle sequence, can be coupled to a cell surface 904 by a releasable linkage 906, such as, for example, a disulfide linker. The nucleic acid molecule 902, can be coupled to a cell surface 904, by conjugation with a cell coupling agent 926, among cell coupling agents 924, 926, 928. The same cell surface 904 may be coupled (e.g., via releasable linkage) to one or more other nucleic acid molecules 918, 920 by conjugation to other cell coupling agents 928, 924, respectively. The nucleic acid molecule 902 may be or comprise a barcode. As noted elsewhere herein, the structure of the barcode may comprise a number of sequence elements. The nucleic acid molecule 902 may comprise a functional sequence 908 that may be used in subsequent processing. For example, the functional sequence 908 may include a universal handle molecule such as one or more of a sequencer specific flow cell attachment sequence (e.g., a P5 sequence for Illumina® sequencing systems) and a sequencing primer sequence (e.g., a R1 primer for Illumina® sequencing systems). The nucleic acid molecule 902 may comprise a barcode sequence 910 for use in barcoding the sample (e.g., DNA, RNA, protein, etc.). In some cases, the barcode sequence 910 can be cell-specific such that the barcode sequence 910 is common to all nucleic acid molecules (e.g., including nucleic acid molecule 902) coupled to the same cell 904. Alternatively or in addition, the barcode sequence 910 can be partition-specific such that the barcode sequence 910 is common to all nucleic acid molecules coupled to one or more cell that are partitioned into the same partition. In some instances, the barcode sequence 910 may be or comprise the composite barcode sequence described elsewhere herein. For example, the barcode sequence 910 may be generated by the split pool ligation methods described herein, and comprise two or more conjugate barcode subsequences, each barcode subsequence added on in each iterative split and pool operation. Other barcode sequences described herein (e.g., 916, 912, 914, 908) may or may not be part of the composite barcode sequence described herein.

In some instances, a nucleic acid molecule attached to the cell (e.g., 902, 918, 920) may comprise a cell coupling agent label, such as an agent barcode sequence which identifies the cell coupling agent or the molecule(s) and/or moiet(ies) (e.g., protein) that the cell coupling agent is configured to bind to. Beneficially, upon sequencing, such agent barcode sequence may identify presence and/or relative quantity of the moiet(ies) on the cell surface of the cell. Any of the functional sequences described herein may be added with a split and pool operation, or independently of any split and pool operation (e.g., such as in bulk).

In some instances, the barcode sequences 908 can comprise a ligation handle sequence 930 for ligation to 910. The barcode sequences 910 can comprise a ligation handle sequence 932 for ligation to 916. The barcode sequence 912 can comprise a ligation handle sequence 936 for ligation to 914. The nucleic acid molecule 902 may comprise a specific priming sequence 912, such as an mRNA specific priming sequence (e.g., poly-T sequence), a targeted priming sequence, and/or a random priming sequence. The nucleic acid molecule 902 may comprise an anchoring sequence 914 to ensure that the specific priming sequence 912 hybridizes at the sequence end (e.g., of the mRNA). For example, the anchoring sequence 914 can include a random short sequence of nucleotides, such as a 1-mer, 2-mer, 3-mer or longer sequence, which can ensure that a poly-T segment is more likely to hybridize at the sequence end of the poly-A tail of the mRNA. The specific priming sequence and/or the anchoring sequence may function as a capture sequence configured to capture an analyte of interest (e.g., mRNA or cDNA derivative thereof).

In some instances, the nucleic acid molecule may comprise one of a cell coupling agent label and a capture sequence. In some instances, the nucleic acid molecule may comprise both a cell coupling agent label and a capture sequence.

The nucleic acid molecule 902 may comprise a unique molecular identifying sequence 916 (e.g., unique molecular identifier (UMI)). In some cases, the unique molecular identifying sequence 916 may comprise from about 5 to about 9 nucleotides. Alternatively, the unique molecular identifying sequence 916 may compress less than about 5 or more than about 9 nucleotides. The unique molecular identifying sequence 916 may be a unique sequence that varies across individual nucleic acid molecules (e.g., 902, 919, 920, etc.) coupled to a single cell (e.g., cell 904). In some cases, the unique molecular identifying sequence 916 may be a random sequence (e.g., such as a random N-mer sequence). For example, the UMI may provide a unique identifier of the starting mRNA molecule that was captured, in order to allow quantitation of the number of original expressed RNA. As will be appreciated, although FIG. 9 shows three nucleic acid molecules 902, 919, 920 coupled to the surface of the cell 904, an individual cell may be coupled to any number of individual nucleic acid molecules, for example, from one to tens to hundreds of thousands or even millions of individual nucleic acid molecules. The respective barcodes for the individual nucleic acid molecules can comprise both common sequence segments or relatively common sequence segments (e.g., 909, 910, 912, etc.) and variable or unique sequence segments (e.g., 916) between different individual nucleic acid molecules coupled to the same cell.

In operation, the barcode bearing cell 904 can be partitioned into a droplet. In some instances, the cell 904 may be lysed. The barcoded nucleic acid molecules 902, 919, 920 can be released from the surface of the cell 904 in the partition. Analytes contained in the cell may also be released. By way of example, in the context of analyzing sample RNA, the poly-T segment (e.g., 912) of one of the released nucleic acid molecules (e.g., 902) can hybridize to the poly-A tail of an mRNA molecule. Reverse transcription may result in a cDNA transcript of the mRNA, but which transcript includes each of the sequence segments 909, 910, 916 of the nucleic acid molecule 902. Because the nucleic acid molecule 902 comprises an anchoring sequence 914, it will more likely hybridize to and prime reverse transcription at the sequence end of the poly-A tail of the mRNA. Within any given partition, all of the cDNA transcripts of the individual mRNA molecules may include a common barcode sequence segment 910. However, the transcripts made from the different mRNA molecules within a given partition may vary at the unique molecular identifying sequence 912 segment (e.g., UMI segment). Beneficially, even following any subsequent amplification of the contents of a given partition, the number of different UMIs can be indicative of the quantity of mRNA originating from a given partition, and thus from the biological particle (e.g., cell). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences may also be used in priming the reverse transcription reaction. Likewise, although described as releasing the barcoded oligonucleotides into the partition, in some cases, the nucleic acid molecules bound to the cell may be used to hybridize and capture the mRNA on the solid phase of the cell, for example, in order to facilitate the separation of the RNA from other cell contents.

Appending or attaching a nucleic acid barcode molecule, or sequence thereof, can be achieved by 5′ or 3′ addition to analyte. In some instances, an appending or attaching is achieved by ligation, hybridization, or any combination thereof. In some cases, template switching can be used to increase the length of a cDNA. In some cases, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner. Switch oligos can include sequences complementary to the additional nucleotides, e.g., polyG. The additional nucleotides (e.g., polyC) on the cDNA can hybridize to the complementary sequence (e.g., polyG) on the switch oligo, whereby the switch oligo can be used by the reverse transcriptase as template to further extend the cDNA. Template switching oligonucleotides may comprise a hybridization region and a template region. The hybridization region can comprise any sequence capable of hybridizing to the target. In some cases, as previously described, the hybridization region comprises a series of G bases to complement the overhanging C bases at the 3′ end of a cDNA molecule. The series of G bases may comprise 1 G base, 2 G bases, 3 G bases, 4 G bases, 5 G bases or more than 5 G bases. The template region can comprise any sequence to be incorporated into the cDNA. In some cases, the template region comprises at least 1 (e.g., at least 2, 3, 4, 5 or more) tag sequences and/or functional sequences. Switch oligos may comprise deoxyribonucleic acids; ribonucleic acids; modified nucleic acids including 2-Aminopurine, 2,6-Diaminopurine (2-Amino-dA), inverted dT, 5-Methyl dC, 2′-deoxyInosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), or any combination. With reference to FIG. 9, a capture sequence (e.g., 912 and/or 914) of a barcode molecule coupled to a surface of the cell may comprise a switch oligo sequence or other sequence configured to capture a cDNA (e.g., comprising polyC sequence) derived from an mRNA.

The cells that can be barcoded include bacteria, archaebacteria, or eukaryotic cells and can constitute a homogeneous cell line or mixed culture. Suitable mammalian cells include those from, e.g., mouse, rat, hamster, primate, and human, both cell lines and primary cultures. Such cells include stem cells, including embryonic stem cells and hemopoietic stem cells, zygotes, fibroblasts, lymphocytes, Chinese hamster ovary (CHO), mouse fibroblasts (NIH3T3), kidney, liver, muscle, and skin cells. Other eukaryotic cells of interest include plant cells, such as maize, rice, wheat, cotton, soybean, sugarcane, tobacco, and arabidopsis; fish, algae, fungi (penicillium, aspergillus, podospora, neurospora, saccharomyces), insect (e.g., baculo lepidoptera), yeast (picchia and saccharomyces, Schizosaccharomyces pombe). Also of interest are many bacterial cell types, both gram-negative and gram-positive, such as Bacillus subtilis, B. licehniformis, B. cereus, Escherichia coli, Streptomyces, Pseudomonas, Salmonella, Actinomycetes and Erwinia.

In some instances, the cells can be stored for long periods of time and used as a reagent for subsequent applications.

In some instances, the composite barcode may comprise an oligonucleotide sequence which facilitates downstream reactions. For example, the downstream reactions are for the evaluation of intracellular analytes of the surface-barcoded cell. The intracellular analyte may comprise a nucleic acid. In some cases, the intracellular analyte may be a macromolecule. The intracellular analyte may comprise DNA. The intracellular analyte may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The intracellular analyte may comprise a protein. The intracellular analyte may comprise a peptide. The intracellular analyte may comprise a polypeptide.

The downstream reactions may comprise, for example reverse transcription of mature mRNAs, capturing specific portions of the transcriptome, priming for DNA polymerases and/or similar enzymes, and the like. For example, the composite barcode sequence may comprise the anchoring sequence 914 with respect to FIG. 9.

In some instances, the downstream molecular biological reactions are for the evaluation of intracellular protein analytes in the cell. In another example, the downstream molecular biological reactions can involve analysis of proteins, protein complexes, proteins with translational modifications, and protein/nucleic acid complexes. Protein targets include peptides, and also include enzymes, hormones, structural components such as viral capsid proteins, and antibodies.

In some cases, the nucleic acid molecule can comprise a functional sequence, for example, for attachment to a sequencing flow cell, such as, for example, a P5 sequence for Illumina® sequencing. In some cases, the nucleic acid molecule or derivative thereof (e.g., oligonucleotide or polynucleotide generated from the nucleic acid molecule) can comprise another functional sequence, such as, for example, a P7 sequence for attachment to a sequencing flow cell for Illumina sequencing. In some cases, the nucleic acid molecule can comprise a barcode sequence. In some cases, the primer can further comprise a unique molecular identifier (UMI). In some cases, the primer can comprise an R1 primer sequence for Illumina sequencing. In some cases, the primer can comprise an R2 primer sequence for Illumina sequencing. Examples of such nucleic acid molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is entirely incorporated herein by reference.

A surface-barcoded cell may be introduced into a partition, such as a droplet of an emulsion or a well, such that the surface-barcoded cell is lysed within the partition and any associated species (e.g., oligonucleotides) are released within the droplet when the appropriate stimulus is applied. The free species (e.g., oligonucleotides, nucleic acid molecules) may interact with other reagents contained in the partition. For example, a surface-barcoded cell comprising an attachment complex that includes a lipid linked, via a disulfide bond, to a universal barcode sequence, may be combined with a reducing agent within a droplet of a water-in-oil emulsion. Within the droplet, the reducing agent can break the various disulfide bonds, resulting in cell degradation and release of the barcode sequence into the aqueous, inner environment of the droplet. In another example, heating of a droplet comprising a cell-bound barcode sequence in basic solution may also result in cell degradation and release of the attached barcode sequence into the aqueous, inner environment of the droplet.

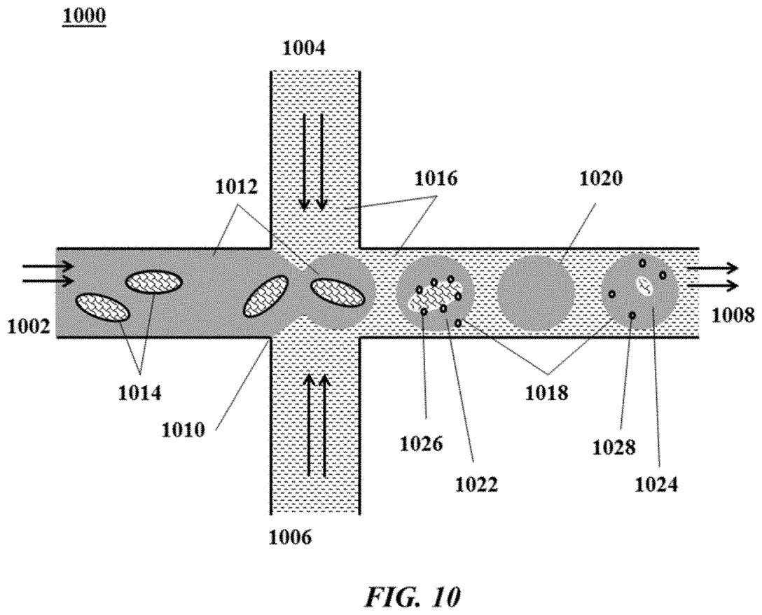

Microfluidic systems of the present disclosure, such as the system 1000 shown in FIG. 10, may be readily used in partitioning surface-indexed cells. In particular, and with reference to FIG. 10, the aqueous fluid 1012 comprising the cells 1014 is flowed into channel junction 1010, where it is partitioned into droplets 1018, 1020 through the flow of non-aqueous fluid 1016. The partitions may also comprise reagents such as lysis solutions 1022 that can cause lysis 1024 of the cells in the respective partitions. Surface-attached barcodes 1026 will therefore be released from the cell and attach to intracellular analyte of the cells. Beneficially, by delivering barcodes on the cell that contains therein the analytes, each partition including the barcoded cell is guaranteed to comprise both the analytes and the barcodes. Further, this prevents the partitioning of only barcodes (without cells) or only cells (without barcodes) in partitions which can result from Poissonian loading, preventing the significant waste of expensive resources (e.g., reagents) and loss of efficiency.

Any suitable number of molecular tag molecules (e.g., primer, barcoded oligonucleotide) can be associated with a surface-barcoded cell such that, upon release from the cell, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing nucleic acid molecule (e.g., oligonucleotide) bearing cells.

A surface-barcoded cell injected or otherwise introduced into a partition may comprise releasably, cleavably, or reversibly attached barcodes. Barcodes can be releasably, cleavably or reversibly attached to the surface-barcoded cells such that barcodes can be released or be releasable through cleavage of a linkage between the barcode molecule and the cell, or released through degradation of the underlying cell itself, allowing the barcodes to be accessed or be accessible by other reagents, or both. In non-limiting examples, cleavage may be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein.

The addition of multiple types of labile bonds to a cell may result in the generation of a cell capable of responding to varied stimuli. Each type of labile bond may be sensitive to an associated stimulus (e.g., chemical stimulus, light, temperature, enzymatic, etc.) such that release of species attached to a cell via each labile bond may be controlled by the application of the appropriate stimulus. Such functionality may be useful in controlled release of species from a cell surface.

As will be appreciated, barcodes that are releasably, cleavably or reversibly attached to the cells described herein include barcodes that are released or releasable through cleavage of a linkage between the barcode molecule and the cell coupling agent, or that are released through degradation of the underlying cell itself, allowing the barcodes to be accessed or accessible by other reagents, or both.

Species may be partitioned in a partition (e.g., droplet) during or subsequent to partition formation. Such species may include, for example, nucleic acid molecules (e.g., oligonucleotides), reagents for a nucleic acid amplification reaction (e.g., primers, polymerases, dNTPs, co-factors (e.g., ionic co-factors), buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species may include one or more enzymes described herein, including without limitation, polymerase, reverse transcriptase, restriction enzymes (e.g., endonuclease), transposase, ligase, proteinase K, DNAse, etc. Such species may include one or more reagents described elsewhere herein (e.g., lysis agents, inhibitors, inactivating agents, chelating agents, stimulus). Alternatively, such species may be introduced into the partition by encapsulation in a bead or linked to a bed.

In some instances, alternative or in addition to barcoding cells (e.g., surfaces thereof), cell beads encapsulating cells (e.g., surfaces thereof) may be barcoded iteratively in split and pool methods described herein.

Functionalized nucleic acid molecules can be attached to cell beads. For example, a cell bead can may comprise any suitable functionalized sequence, such as those described elsewhere herein. For example, functionalized nucleic acid molecules may comprise a sequence configured to hybridize to a nucleic acid molecule (e.g., a poly-T sequence, a random N-mer sequence, a sequence complementary to a cellular nucleic acid sequence), a primer sequence, a template switching oligonucleotide (TSO) sequence, a barcode sequence, a unique molecular index (UMI) sequence, a sequencing primer sequence (or a partial sequencing primer sequence, such as a partial R1 and/or R2 sequence), and/or one or more adaptor sequences, such as a sequence configured to attach to the flow cell of a sequencer (e.g., P5, P7), etc. In some embodiments, the nucleic acid molecules attached to a cell bead are single-stranded nucleic acid molecules. In some embodiments, the nucleic acid molecules attached to a cell bead are double-stranded nucleic acid molecules. In some embodiments, the nucleic acid molecules attached to a cell bead are partially double-stranded nucleic acid molecules.

In some instances, alternative or in addition to barcoding cells (e.g., surfaces thereof), analytes therein may be barcoded iteratively in split and pool methods described herein. Such systems and methods may utilize cell beads. A cell may be processed to generate a cell bead. This may be performed by providing the cell in a partition, such as a droplet. Systems and methods for generating a cell bead, such as by encapsulating and polymerizing a cell, are described elsewhere herein. The cell may be lysed to release one or more analytes in the cell bead, such as a first analyte, a second analyte, and a third analyte. Alternatively, the cell may be lysed prior to formation of the cell bead, and a cell bead generated with the lysed contents thereafter. For example, the cell is provided in a droplet and lysed, and the droplet is subsequently subjected to polymerization to generate the cell bead. A plurality of such cell beads may be partitioned into a plurality of droplets such as to allow for overloading of cell beads, wherein a partition may co-partition multiple cell beads. A plurality of partition specific barcodes may be introduced into each partition, such that for a partition, the analytes of each respective cell bead in the partition receives the partition specific barcode for that partition. The partition specific barcode may be appended or otherwise attached to the analytes. In some instances, the partition specific barcode may have binding specificity to one or more analytes of interest (e.g., protein, nucleic acids, etc.). Then, the contents of the partitions may be pooled to provide the plurality of cell beads. In a next iteration, the plurality of cell beads may be split into partitions again, such as to allow for overloading of cell beads. A plurality of partition specific barcodes (for the second round of partitions) may be introduced into each partition, such that for a partition, the analytes of each respective cell bead in the partition receives the partition specific barcode for that partition. The partition specific barcode may be appended or otherwise attached to the analytes or to the already appended barcode (received in the first round). In some instances, the partition specific barcode may have binding specificity to one or more analytes of interest (e.g., protein, nucleic acids, etc.) and/or to another barcode molecule (e.g., the partition specific barcode molecule of the first round).

The above split and pool (with overloading) of cell beads may be repeated for any number of cycles, wherein a partition-specific barcode is provided to a cell bead at each round of partitioning. In some instances, the number of cycles may be selected to minimize a probability that a final combination of barcodes resulting in a cell bead is not unique among a plurality of cell beads. The overloading of cell beads in each iteration enables multiple cells to be processed per partition, and at greater throughput than can usefully be achieved with normal sub-Poisson cell loading. The multiple rounds of partitioning and barcoding may enable high multiplexing with unique cell identifiability based on the combination of barcodes. The analytes in the cell bead may be attached to a partition-specific barcode received at each split and pool cycle. For example, the analytes may be attached to a composite barcode sequence comprising conjugated barcode subsequences, as described elsewhere herein. The barcode subsequences may be adjacent in an order of partitioning. In another example, the analytes may be attached to a combination of barcode molecules, such as at different locations (e.g., at opposing ends of a nucleic acid sequence, etc.).

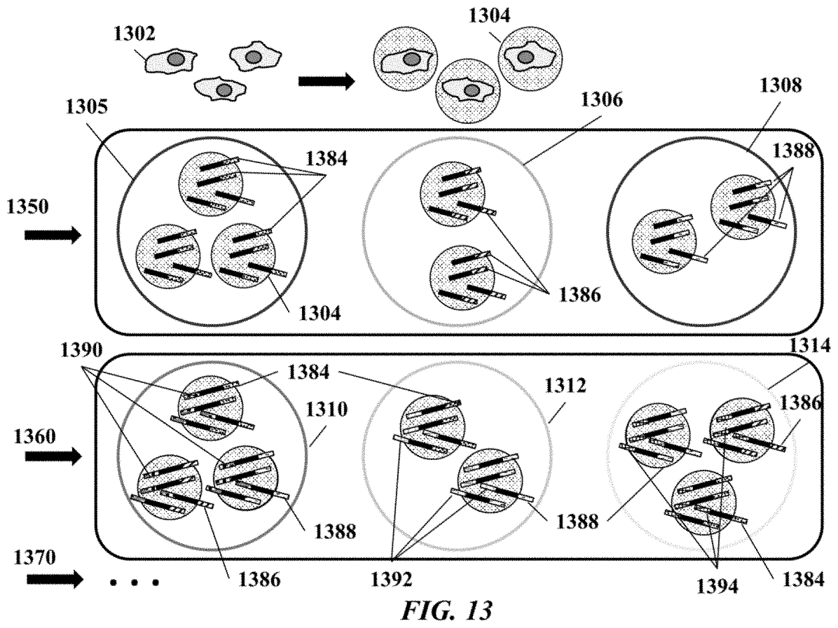

Subsequent to split pooling at least 40%, 50%, 60%, 70%, 80%, 90%, 95% of respective cells of a plurality of cells that are processed may comprise a unique composite barcode sequence. In certain instances, the percent of processed cells that comprise a unique composite barcode sequence is about 10% to about 100%. In certain instances, the percent of processed cells that comprise a unique composite barcode sequence is about 10% to about 25%, about 10% to about 50%, about 10% to about 60%, about 10% to about 70%, about 10% to about 80%, about 10% to about 90%, about 10% to about 100%, about 25% to about 50%, about 25% to about 60%, about 25% to about 70%, about 25% to about 80%, about 25% to about 90%, about 25% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100%. In certain instances, the percent of processed cells that comprise a unique composite barcode sequence is about 10%, about 25%, about 50%, about 60%, about 70%, about 80%, about 90%, or about 100%. In certain instances, the percent of processed cells that comprise a unique composite barcode sequence is at least about 10%, about 25%, about 50%, about 60%, about 70%, about 80%, or about 90%. FIG. 13 illustrates a process flow of iterative cell bead barcoding. A plurality of cells 1302 can be processed to generate a plurality of cell beads 1304. In some instances, the cells may be lysed to release one or more analytes in the cell beads. In some instances, the cells may be lysed prior to formation of the cell beads, and cell beads generated with the lysed contents thereafter. A cell bead may physically retain contents (e.g., analytes) therein. The plurality of cell beads 1304 may be partitioned 1350 into a first plurality of partitions, such as a first partition 1305, a second partition 1306, and a third partition 1308. Partitions may be overloaded with cell beads such that a partition contains more than one cell bead of the plurality of cell beads 1304. Partition specific barcodes may be delivered to the respective cell beads in each partition. For example, three cell beads encapsulated in the first partition 1305 may each receive a first partition specific barcode 1384. Analytes in the three cell beads may be attached to the first partition specific barcode 1384. Two cell beads encapsulated in the second partition 1306 may each receive a second partition specific barcode 1386. Analytes in the two cell beads may be attached to the second partition specific barcode 1386. Two cell beads encapsulated in the third partition 1308 may each receive a third partition specific barcode 1388. Analytes in the two cell beads may be attached to the third partition specific barcode 1388.

Thereafter, the first plurality of partitions, or cell beads therein, may be pooled and re-partitioned 1360 into a second plurality of partitions, such as including a fourth partition 1310, a fifth partition 1312, and a sixth partition 1314. Partitions may be overloaded with cell beads such that a partition contains more than one cell bead of the plurality of cell beads 1304. Partition specific barcodes may be delivered to the respective cell beads in each partition. For example, three cell beads encapsulated in the fourth partition 1310, derived from each of the first partition 1305, the second partition 1306, and the third partition 1308, may each receive a fourth partition specific barcode 1390. Analytes in the three cell beads may be attached to the fourth partition specific barcode 1390, such that analytes in the cell bead that derived from the first partition 1305 is attached to both the first partition specific barcode 1384 and the fourth partition specific barcode 1390, analytes in the cell bead that derived from the second partition 1306 is attached to both the second partition specific barcode 1386 and the fourth partition specific barcode 1390, and analytes in the cell bead that derived from the third partition 1308 is attached to both the third partition specific barcode 1388 and the fourth partition specific barcode 1390. For example, two cell beads encapsulated in the fifth partition 1312, derived from each of the first partition 1305 and the third partition 1308, may each receive a fifth partition specific barcode 1392. Analytes in the two cell beads may be attached to the fifth partition specific barcode 1392, such that analytes in the cell bead that derived from the first cell bead 1305 is attached to both the first partition specific barcode 1384 and the fifth partition specific barcode 1392, and analytes in the cell bead that derived from the third partition 1308 is attached to both the third partition specific barcode 1388 and the fifth partition specific barcode 1392. For example, three cell beads encapsulated in the sixth partition 1314, derived from each of the first partition 1305, the second partition 1306, and the third partition 1308, may each receive a sixth partition specific barcode 1394. Analytes in the three cell beads may be attached to the sixth partition specific barcode 1394, such that analytes in the cell bead that derived from the first partition 1305 is attached to both the first partition specific barcode 1384 and the sixth partition specific barcode 1394, analytes in the cell bead that derived from the second partition 1306 is attached to both the second partition specific barcode 1386 and the sixth partition specific barcode 1394, and analytes in the cell bead that derived from the third partition 1308 is attached to both the third partition specific barcode 1388 and the sixth partition specific barcode 1394. Thereafter, the second plurality of partitions, or cell beads therein, may be pooled and re-partitioned 1370 any number of times into a subsequent plurality of partitions such that analytes in a cell bead receive a partition specific barcode at each cycle of overloaded partitioning. Beneficially, the unique combination of barcodes attached to the analytes (common to the analytes in the same cell bead) may distinguish the analytes from analytes derived from other cell beads (or cells).

Cell beads may contain one or more attached composite barcode molecules comprising barcode sequences. The barcode sequences associated with a single cell bead may be identical or comprise identical sequences in part. In some cases, the barcode sequences associated with a single cell bead may be different or comprise different (e.g., unique) sequences in part. In some cases, a cell bead may be attached to about 1, 5, 10, 50, 100, 500, 1000, 5000, 10000, 20000, 50000, 100000, 500000, 1000000, 5000000, 10000000, 50000000, 100000000, 500000000, 1000000000, 5000000000, 10000000000, 50000000000, or 100000000000 composite barcode molecules. In some cases, a cell bead may be associated with at least about 1, 5, 10, 50, 100, 500, 1000, 5000, 10000, 20000, 50000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, 2000000000, 3000000000, 4000000000, 5000000000, 6000000000, 7000000000, 8000000000, 9000000000, 10000000000, 20000000000, 30000000000, 40000000000, 50000000000, 60000000000, 70000000000, 80000000000, 90000000000, 100000000000 or more composite barcode molecules. In some cases, a cell bead may be associated with less than about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 500000, 1000000, 5000000, 10000000, 50000000, 1000000000, 5000000000, 10000000000, 50000000000, or 100000000000 composite barcode molecules.

An individual barcode library, e.g., a barcoded library of cells or cell beads, may comprise one or more barcoded cells or cell beads. In some cases, an individual barcode library may comprise about 1, 5, 10, 50, 100, 500, 1000, 5000, 10000, 20000, 50000, 100000, 500000, 1000000, 5000000, 10000000, 50000000, 100000000, 500000000, 1000000000, 5000000000, 10000000000, 50000000000, or 100000000000 individual barcoded cells or cell beads. In some cases, a library may comprise at least about 1, 5, 10, 50, 100, 500, 1000, 5000, 10000, 20000, 50000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, 2000000000, 3000000000, 4000000000, 5000000000, 6000000000, 7000000000, 8000000000, 9000000000, 10000000000, 20000000000, 30000000000, 40000000000, 50000000000, 60000000000, 70000000000, 80000000000, 90000000000, 100000000000 or more individual barcoded cells or cell beads. In some cases, each library may comprise less than about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 500000, 1000000, 5000000, 10000000, 50000000, 1000000000, 5000000000, 10000000000, 50000000000, or 100000000000 individual barcoded cells or cell beads. The barcoded cells or cell beads within the library may have the same sequences or different sequences.

In some cases, cells or cell beads in the barcode library may have a unique barcode sequence. However, the number of cells or cell beads with unique barcode sequences within a barcode library may be limited by combinatorial limits. For example, using four different nucleotides, if a barcode is 12 nucleotides in length, then the number of unique constructs may be limited to 412=16777216 unique constructs. If barcode libraries comprise many more cells than 16777216, in such cases there may be some libraries with multiple copies of the same barcode. In some cases, the percentage of multiple copies of the same barcode within a given library may be 1%, 2%, 3%, 4%, 5%, 6%, 7%, 8%, 9%, 10%, 15%, 20%, 25%, 30%, 40%, or 50%. In some cases, the percentage of multiple copies of the same barcode within a given library may be more than 1%, 2%, 3%, 4%, 5%, 6%, 7%, 8%, 9%, 10%, 15%, 20%, 25%, 30%, 40%, 50% or more. In some cases, the percentage of multiple copies of the same barcode within a given library may be less than 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, or 50%.

In some cases, each cell or cell bead may comprise one unique barcode sequence but multiple different random N-mers. In some cases, each cell or cell bead may have one or more different random N-mers. Again, the number of cells or cell beads with different random N-mers within a barcode library may be limited by combinatorial limits. For example, using four different nucleotides, if an N-mer sequence is 12 nucleotides in length, then the number of different constructs may be limited to 412=16777216 different constructs. If barcode libraries may comprise many more cells or cell beads than 16777216, in such cases there may be some libraries with multiple copies of the same N-mer sequence. In some cases, the percentage of multiple copies of the same N-mer sequence within a given library may be 1%, 2%, 3%, 4%, 5%, 6%, 7%, 8%, 9%, 10%, 15%, 20%, 25%, 30%, 40%, or 50%. In some cases, the percentage of multiple copies of the same N-mer sequence within a given library may be more than 1%, 2%, 3%, 4%, 5%, 6%, 7%, 8%, 9%, 10%, 15%, 20%, 25%, 30%, 40%, 50% or more. In some cases, the percentage of multiple copies of the same N-mer sequence within a given library may be less than 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, or 50%.

Accordingly, provided herein are methods of processing cells, comprising: (a) partitioning a plurality of cells and a plurality of nucleic acid barcode molecules comprising barcode sequences into a plurality of partitions, wherein a partition of the plurality of partitions comprises a first cell of the plurality of cells and a first barcode molecule of the plurality of nucleic acid barcode molecules, wherein the first barcode molecule comprises a first barcode sequence that is unique to the partition among the plurality of partitions; (b) in the partition, attaching the first barcode molecule to a surface of the first cell, wherein the first barcode sequence is different than other barcode sequences in other partitions of the plurality of partitions; (c) pooling cells from the plurality of cells, including the first cell, from the plurality of partitions; (d) re-partitioning the plurality of cells and an additional plurality of nucleic acid barcode molecules into an additional plurality of partitions, wherein a partition of the additional plurality of partitions comprises the first cell and an additional barcode molecule comprising an additional barcode sequence that is unique to the partition of the additional plurality of partitions; (e) in the partition of (d), coupling the additional barcode molecule to the first barcode molecule, thereby indexing the first cell with a nucleic acid composite barcode molecule comprising a composite barcode sequence comprising the first barcode sequence and additional barcode sequence, wherein the nucleic acid composite barcode molecule comprises a capture sequence configured to capture an analyte.

In some instances, subsequent to (e), (c)-(e) are repeated N times, wherein N is an integer greater than or equal to 1, and wherein the composite barcode sequence comprises the first barcode sequence and N+1 additional barcode sequences. The method of claim 2, wherein a (N+1)th barcode molecule is configured to couple to a Nth barcode nucleic acid molecule. In some instances, the method further comprises, prior to (a), coupling a cell coupling agent to the surface of the first cell, wherein the cell coupling agent is coupled to an oligonucleotide configured to couple to the first barcode molecule. In some instances, prior to (a), a plurality of cell coupling agents are coupled to the surface of the first cell, wherein the plurality of cell coupling agents comprises the cell coupling agent.

In some instances, the first barcode molecule is configured to couple to a second barcode molecule. In some instances, the first barcode molecule is configured to couple to one or more splint molecules, wherein the one or more splint molecules are configured to couple to the second barcode molecule. In some instances, the cell coupling agent comprises a peptide or polypeptide. In some instances, the peptide or polypeptide is configured to couple to an antigen on the cell surface of the first cell. In some instances, the peptide or polypeptide is configured to couple to a carbohydrate group on a cell membrane of the first cell. In some instances, the cell coupling agent comprises a lipid molecule, wherein the lipid molecule is configured to embed into a cell membrane of the first cell and the oligonucleotide is configured to couple to the first barcode molecule. In some instances, the cell coupling agent comprises a disulfide bond.

In some instances, the method further comprises, subsequent to indexing the first cell with the nucleic acid composite barcode molecule comprising the composite barcode sequence, partitioning the first cell into a third partition. In some instances, the method further comprises coupling the nucleic acid composite barcode molecule comprising the composite barcode sequence to the analyte, wherein the analyte is a cellular analyte of the first cell, thereby generating a barcoded analyte. In some instances, the method further comprises, determining a sequence of the barcoded analyte, wherein the determined sequence of the barcoded analyte comprises the composite barcode sequence or complement thereof. In some instances, the method further comprises, using the composite barcode sequence or complement thereof to identify the analyte as a cellular analyte of the first cell. In some instances, the method further comprises, lysing the cell in the third partition to release the analyte. In some instances, the analyte is selected from a ribonucleic acid (RNA) molecule, a DNA molecule, a gDNA molecule, a protein, or any combination thereof In some instances, the RNA molecule is a messenger RNA (mRNA) molecule.

In some instances, the method further comprises, releasing the cell coupling agent from the cell surface or releasing the oligonucleotide from the cell coupling agent. In some instances, the releasing the cell coupling agent comprises cleaving a disulfide bond. In some instances, the partition is a droplet. In some instances, the partition is a well. In some instances, the partition is a microwell or a nanowell. In some instances, the partition is the nanowell, wherein the nanowell is from a nanowell array. In some instances, the microwell is from a 96-well plate or a 384-well plate. In some instances, subsequent to (a), the partition comprises more than one cell.

In some instances, subsequent to (e), (c)-(e) are repeated 2 times, and wherein in (d) the additional plurality of partitions comprises at least 96 partitions. In some instances, subsequent to (e), (c)-(e) are repeated 3 times. In some instances, (a)-(e) are performed for each cell of the plurality of cells, and wherein subsequent to (e), at least 99% of respective cells of the plurality of cells each comprises a respective composite barcode sequence that is unique to the respective cells among the plurality of cells.

### Systems and Methods for Sample Compartmentalization

In an aspect, the systems and methods described herein provide for the compartmentalization, depositing, or partitioning of one or more particles (e.g., biological particles, intracellular analytes of biological particles, beads, reagents, etc.) into discrete compartments or partitions (referred to interchangeably herein as partitions), where each partition maintains separation of its own contents from the contents of other partitions. The partition can be a droplet in an emulsion. A partition may comprise one or more other partitions.

A partition may include one or more particles. A partition may include one or more types of particles. For example, a partition of the present disclosure may comprise one or more biological particles and/or intracellular analytes thereof. A partition may comprise one or more gel beads. A partition may comprise one or more cell beads. A partition may include a single gel bead, a single cell bead, or both a single cell bead and single gel bead. A partition may include one or more reagents. Alternatively, a partition may be unoccupied. For example, a partition may not comprise a bead. A cell bead can be a biological particle and/or one or more of its intracellular analytes encased inside of a gel or polymer matrix, such as via polymerization of a droplet containing the biological particle and precursors capable of being polymerized or gelled. Unique identifiers, such as barcodes, may be injected into the droplets previous to, subsequent to, or concurrently with droplet generation, such as via a microcapsule (e.g., bead), as described elsewhere herein. Microfluidic channel networks (e.g., on a chip) can be utilized to generate partitions as described herein. Alternative mechanisms may also be employed in the partitioning of individual biological particles, including porous membranes through which aqueous mixtures of cells are extruded into non-aqueous fluids.

The partitions can be flowable within fluid streams. The partitions may comprise, for example, micro-vesicles that have an outer barrier surrounding an inner fluid center or core. In some cases, the partitions may comprise a porous matrix that is capable of entraining and/or retaining materials within its matrix. The partitions can be droplets of a first phase within a second phase, wherein the first and second phases are immiscible. For example, the partitions can be droplets of aqueous fluid within a non-aqueous continuous phase (e.g., oil phase). In another example, the partitions can be droplets of a non-aqueous fluid within an aqueous phase. In some examples, the partitions may be provided in a water-in-oil emulsion or oil-in-water emulsion. A variety of different vessels are described in, for example, U.S. Patent Application Publication No. 2014/0155295, which is entirely incorporated herein by reference for all purposes. Emulsion systems for creating stable droplets in non-aqueous or oil continuous phases are described in, for example, U.S. Patent Application Publication No. 2010/0105112, which is entirely incorporated herein by reference for all purposes.

In the case of droplets in an emulsion, allocating individual particles to discrete partitions may in one non-limiting example be accomplished by introducing a flowing stream of particles in an aqueous fluid into a flowing stream of a non-aqueous fluid, such that droplets are generated at the junction of the two streams. Fluid properties (e.g., fluid flow rates, fluid viscosities, etc.), particle properties (e.g., volume fraction, particle size, particle concentration, etc.), microfluidic architectures (e.g., channel geometry, etc.), and other parameters may be adjusted to control the occupancy of the resulting partitions (e.g., number of biological particles per partition, number of beads per partition, etc.). For example, partition occupancy can be controlled by providing the aqueous stream at a certain concentration and/or flow rate of particles. To generate single biological particle partitions, the relative flow rates of the immiscible fluids can be selected such that, on average, the partitions may contain less than one biological particle per partition in order to ensure that those partitions that are occupied are primarily singly occupied. In some cases, partitions among a plurality of partitions may contain at most one biological particle (e.g., bead, DNA, cell or cellular material). In some cases, the various parameters (e.g., fluid properties, particle properties, microfluidic architectures, etc.) may be selected or adjusted such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

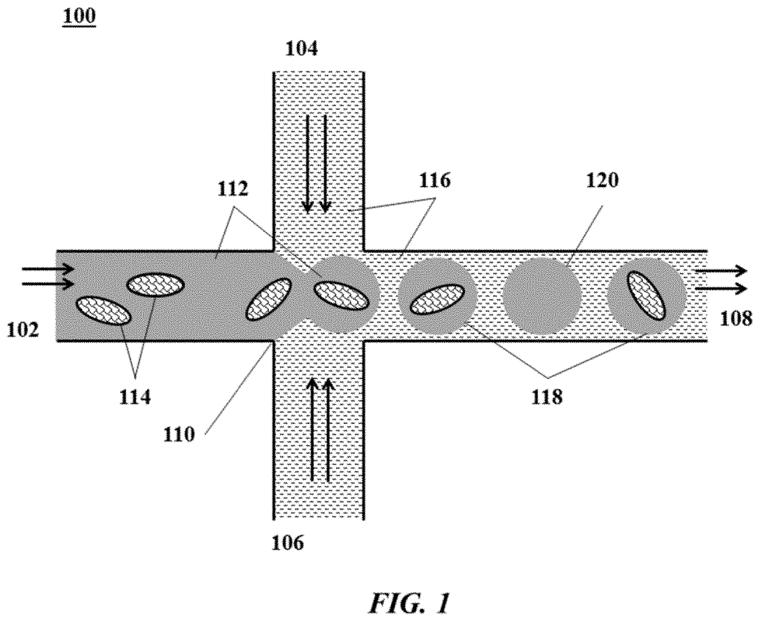

FIG. 1 shows an example of a microfluidic channel structure 100 for partitioning individual biological particles. The channel structure 100 can include channel segments 102, 104, 106 and 108 communicating at a channel junction 110. In operation, a first aqueous fluid 112 that includes suspended biological particles (or cells) 114 may be transported along channel segment 102 into junction 110, while a second fluid 116 that is immiscible with the aqueous fluid 112 is delivered to the junction 110 from each of channel segments 104 and 106 to create discrete droplets 118, 120 of the first aqueous fluid 112 flowing into channel segment 108, and flowing away from junction 110. The channel segment 108 may be fluidically coupled to an outlet reservoir where the discrete droplets can be stored and/or harvested. A discrete droplet generated may include an individual biological particle 114 (such as droplets 118). A discrete droplet generated may include more than one individual biological particle 114 (not shown in FIG. 1). A discrete droplet may contain no biological particle 114 (such as droplet 120). Each discrete partition may maintain separation of its own contents (e.g., individual biological particle 114) from the contents of other partitions.

The second fluid 116 can comprise an oil, such as a fluorinated oil, that includes a fluorosurfactant for stabilizing the resulting droplets, for example, inhibiting subsequent coalescence of the resulting droplets 118, 120. Examples of particularly useful partitioning fluids and fluorosurfactants are described, for example, in U.S. Patent Application Publication No. 2010/0105112, which is entirely incorporated herein by reference for all purposes.

As will be appreciated, the channel segments described herein may be coupled to any of a variety of different fluid sources or receiving components, including reservoirs, tubing, manifolds, or fluidic components of other systems. As will be appreciated, the microfluidic channel structure 100 may have other geometries. For example, a microfluidic channel structure can have more than one channel junction. For example, a microfluidic channel structure can have 2, 3, 4, or 5 channel segments each carrying particles (e.g., biological particles, cell beads, and/or gel beads) that meet at a channel junction. Fluid may be directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

The generated droplets may comprise two subsets of droplets: (1) occupied droplets 118, containing one or more biological particles 114, and (2) unoccupied droplets 120, not containing any biological particles 114. Occupied droplets 118 may comprise singly occupied droplets (having one biological particle) and multiply occupied droplets (having more than one biological particle). As described elsewhere herein, in some cases, the majority of occupied partitions can include no more than one biological particle per occupied partition and some of the generated partitions can be unoccupied (of any biological particle). In some cases, though, some of the occupied partitions may include more than one biological particle. In some cases, the partitioning process may be controlled such that fewer than about 25% of the occupied partitions contain more than one biological particle, and in many cases, fewer than about 20% of the occupied partitions have more than one biological particle, while in some cases, fewer than about 10% or even fewer than about 5% of the occupied partitions include more than one biological particle per partition.

In some cases, it may be desirable to minimize the creation of excessive numbers of empty partitions, such as to reduce costs and/or increase efficiency. While this minimization may be achieved by providing a sufficient number of biological particles (e.g., biological particles 114) at the partitioning junction 110, such as to ensure that at least one biological particle is encapsulated in a partition, the Poissonian distribution may expectedly increase the number of partitions that include multiple biological particles. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, the flow of one or more of the biological particles (e.g., in channel segment 102), or other fluids directed into the partitioning junction (e.g., in channel segments 104, 106) can be controlled such that, in many cases, no more than about 50% of the generated partitions, no more than about 25% of the generated partitions, or no more than about 10% of the generated partitions are unoccupied. These flows can be controlled so as to present a non-Poissonian distribution of single-occupied partitions while providing lower levels of unoccupied partitions. The above noted ranges of unoccupied partitions can be achieved while still providing any of the single occupancy rates described above. For example, in many cases, the use of the systems and methods described herein can create resulting partitions that have multiple occupancy rates of less than about 25%, less than about 20%, less than about 15%, less than about 10%, and in many cases, less than about 5%, while having unoccupied partitions of less than about 50%, less than about 40%, less than about 30%, less than about 20%, less than about 10%, less than about 5%, or less.

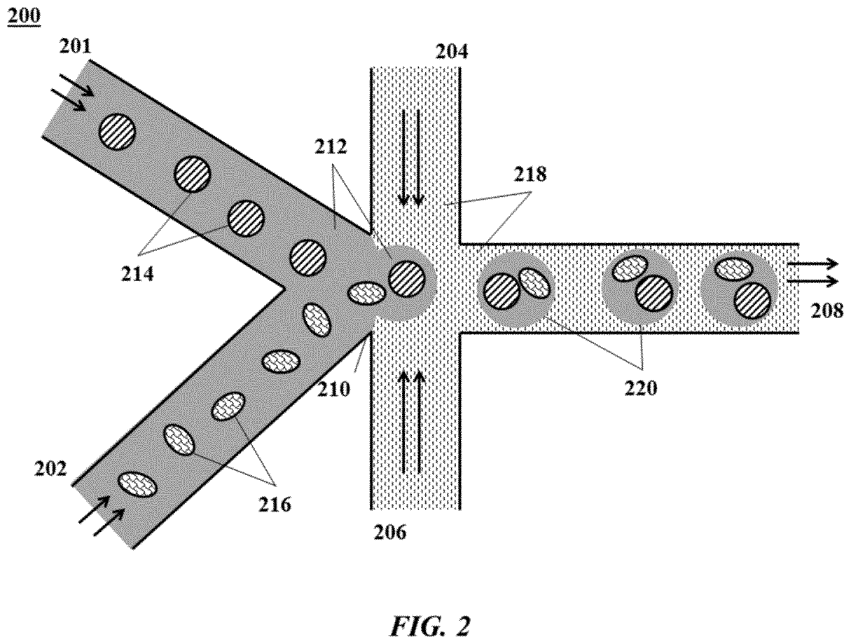

As will be appreciated, the above-described occupancy rates are also applicable to partitions that include both biological particles and additional reagents, including, but not limited to, microcapsules or beads (e.g., gel beads) carrying barcoded nucleic acid molecules (e.g., oligonucleotides) (described in relation to FIG. 2). The occupied partitions (e.g., at least about 10%, 20%, 30%, 40%, 50%, 60%, 70%, 80%, 90%, 95%, or 99% of the occupied partitions) can include both a microcapsule (e.g., bead) comprising barcoded nucleic acid molecules and a biological particle.

In another aspect, in addition to or as an alternative to droplet based partitioning, biological particles may be encapsulated within a microcapsule that comprises an outer shell, layer or porous matrix in which is entrained one or more individual biological particles or small groups of biological particles. The microcapsule may include other reagents. Encapsulation of biological particles may be performed by a variety of processes. Such processes may combine an aqueous fluid containing the biological particles with a polymeric precursor material that may be capable of being formed into a gel or other solid or semi-solid matrix upon application of a particular stimulus to the polymer precursor. Such stimuli can include, for example, thermal stimuli (e.g., either heating or cooling), photo-stimuli (e.g., through photo-curing), chemical stimuli (e.g., through crosslinking, polymerization initiation of the precursor (e.g., through added initiators)), mechanical stimuli, or a combination thereof.

Preparation of microcapsules comprising biological particles may be performed by a variety of methods. For example, air knife droplet or aerosol generators may be used to dispense droplets of precursor fluids into gelling solutions in order to form microcapsules that include individual biological particles or small groups of biological particles. Likewise, membrane based encapsulation systems may be used to generate microcapsules comprising encapsulated biological particles as described herein. Microfluidic systems of the present disclosure, such as that shown in FIG. 1, may be readily used in encapsulating cells as described herein. In particular, and with reference to FIG. 1, the aqueous fluid 112 comprising (i) the biological particles 114 and (ii) the polymer precursor material (not shown) is flowed into channel junction 110, where it is partitioned into droplets 118, 120 through the flow of non-aqueous fluid 116. In the case of encapsulation methods, non-aqueous fluid 116 may also include an initiator (not shown) to cause polymerization and/or crosslinking of the polymer precursor to form the microcapsule that includes the entrained biological particles. Examples of polymer precursor/initiator pairs include those described in U.S. Patent Application Publication No. 2014/0378345, which is entirely incorporated herein by reference for all purposes.

For example, in the case where the polymer precursor material comprises a linear polymer material, such as a linear polyacrylamide, PEG, or other linear polymeric material, the activation agent may comprise a cross-linking agent, or a chemical that activates a cross-linking agent within the formed droplets. Likewise, for polymer precursors that comprise polymerizable monomers, the activation agent may comprise a polymerization initiator. For example, in certain cases, where the polymer precursor comprises a mixture of acrylamide monomer with a N,N′-bis-(acryloyl)cystamine (BAC) comonomer, an agent such as tetraethylmethylenediamine (TEMED) may be provided within the second fluid streams 116 in channel segments 104 and 106, which can initiate the copolymerization of the acrylamide and BAC into a cross-linked polymer network, or hydrogel.

Upon contact of the second fluid stream 116 with the first fluid stream 112 at junction 110, during formation of droplets, the TEMED may diffuse from the second fluid 116 into the aqueous fluid 112 comprising the linear polyacrylamide, which will activate the crosslinking of the polyacrylamide within the droplets 118, 120, resulting in the formation of gel (e.g., hydrogel) microcapsules, as solid or semi-solid beads or particles entraining the cells 114. Although described in terms of polyacrylamide encapsulation, other ‘activatable’ encapsulation compositions may also be employed in the context of the methods and compositions described herein. For example, formation of alginate droplets followed by exposure to divalent metal ions (e.g., Ca²⁺ ions), can be used as an encapsulation process using the described processes. Likewise, agarose droplets may also be transformed into capsules through temperature based gelling (e.g., upon cooling, etc.).

In some cases, encapsulated biological particles can be selectively releasable from the microcapsule, such as through passage of time or upon application of a particular stimulus, that degrades the microcapsule sufficiently to allow the biological particles (e.g., cell), or its other contents to be released from the microcapsule, such as into a partition (e.g., droplet). For example, in the case of the polyacrylamide polymer described above, degradation of the microcapsule may be accomplished through the introduction of an appropriate reducing agent, such as DTT or the like, to cleave disulfide bonds that cross-link the polymer matrix. See, for example, U.S. Patent Application Publication No. 2014/0378345, which is entirely incorporated herein by reference for all purposes.

The biological particle can be subjected to other conditions sufficient to polymerize or gel the precursors. The conditions sufficient to polymerize or gel the precursors may comprise exposure to heating, cooling, electromagnetic radiation, and/or light. The conditions sufficient to polymerize or gel the precursors may comprise any conditions sufficient to polymerize or gel the precursors. Following polymerization or gelling, a polymer or gel may be formed around the biological particle. The polymer or gel may be diffusively permeable to chemical or biochemical reagents. The polymer or gel may be diffusively impermeable to intracellular analytes of the biological particle. In this manner, the polymer or gel may act to allow the biological particle to be subjected to chemical or biochemical operations while spatially confining the intracellular analytes to a region of the droplet defined by the polymer or gel. The polymer or gel may include one or more of disulfide cross-linked polyacrylamide, agarose, alginate, polyvinyl alcohol, polyethylene glycol (PEG)-diacrylate, PEG-acrylate, PEG-thiol, PEG-azide, PEG-alkyne, other acrylates, chitosan, hyaluronic acid, collagen, fibrin, gelatin, or elastin. The polymer or gel may comprise any other polymer or gel.

The polymer or gel may be functionalized to bind to targeted analytes, such as nucleic acids, proteins, carbohydrates, lipids or other analytes. The polymer or gel may be polymerized or gelled via a passive mechanism. The polymer or gel may be stable in alkaline conditions or at elevated temperature. The polymer or gel may have mechanical properties similar to the mechanical properties of the bead. For instance, the polymer or gel may be of a similar size to the bead. The polymer or gel may have a mechanical strength (e.g., tensile strength) similar to that of the bead. The polymer or gel may be of a lower density than an oil. The polymer or gel may be of a density that is roughly similar to that of a buffer. The polymer or gel may have a tunable pore size. The pore size may be chosen to, for instance, retain denatured nucleic acids. The pore size may be chosen to maintain diffusive permeability to exogenous chemicals such as sodium hydroxide (NaOH) and/or endogenous chemicals such as inhibitors. The polymer or gel may be biocompatible. The polymer or gel may maintain or enhance cell viability. The polymer or gel may be biochemically compatible. The polymer or gel may be polymerized and/or depolymerized thermally, chemically, enzymatically, and/or optically.

The polymer may comprise poly(acrylamide-co-acrylic acid) crosslinked with disulfide linkages. The preparation of the polymer may comprise a two-step reaction. In the first activation step, poly(acrylamide-co-acrylic acid) may be exposed to an acylating agent to convert carboxylic acids to esters. For instance, the poly(acrylamide-co-acrylic acid) may be exposed to 4-(4,6-dimethoxy-1,3,5-triazin-2-yl)-4-methylmorpholinium chloride (DMTMM). The polyacrylamide-co-acrylic acid may be exposed to other salts of 4-(4,6-dimethoxy-1,3,5-triazin-2-yl)-4-methylmorpholinium. In the second cross-linking step, the ester formed in the first step may be exposed to a disulfide crosslinking agent. For instance, the ester may be exposed to cystamine (2,2′-dithiobis(ethylamine)). Following the two steps, the biological particle may be surrounded by polyacrylamide strands linked together by disulfide bridges. In this manner, the biological particle may be encased inside of or comprise a gel or matrix (e.g., polymer matrix) to form a “cell bead.” A cell bead can contain biological particles (e.g., a cell) or intracellular analytes (e.g., RNA, DNA, proteins, etc.) of biological particles. A cell bead may include a single cell or multiple cells, or a derivative of the single cell or multiple cells. For example after lysing and washing the cells, inhibitory components from cell lysates can be washed away and the intracellular analytes can be bound as cell beads. Systems and methods disclosed herein can be applicable to both cell beads (and/or droplets or other partitions) containing biological particles and cell beads (and/or droplets or other partitions) containing intracellular analytes of biological particles.

Encapsulated biological particles can provide certain potential advantages of being more storable and more portable than droplet-based partitioned biological particles. Furthermore, in some cases, it may be desirable to allow biological particles to incubate for a select period of time before analysis, such as in order to characterize changes in such biological particles over time, either in the presence or absence of different stimuli. In such cases, encapsulation may allow for longer incubation than partitioning in emulsion droplets, although in some cases, droplet partitioned biological particles may also be incubated for different periods of time, e.g., at least 10 seconds, at least 30 seconds, at least 1 minute, at least 5 minutes, at least 10 minutes, at least 30 minutes, at least 1 hour, at least 2 hours, at least 5 hours, or at least 10 hours or more. The encapsulation of biological particles may constitute the partitioning of the biological particles into which other reagents are co-partitioned. Alternatively or in addition, encapsulated biological particles may be readily deposited into other partitions (e.g., droplets) as described above.

described herein, one or more processes may be performed in a partition, which may be a well. The well may be a well of a plurality of wells of a substrate, such as a microwell of a microwell array or plate, or the well may be a microwell or microchamber of a device (e.g., microfluidic device) comprising a substrate. The well may be a well of a well array or plate, or the well may be a well or chamber of a device (e.g., fluidic device). Accordingly, the wells or microwells may assume an “open” configuration, in which the wells or microwells are exposed to the environment (e.g., contain an open surface) and are accessible on one planar face of the substrate, or the wells or microwells may assume a “closed” or “sealed” configuration, in which the microwells are not accessible on a planar face of the substrate. In some instances, the wells or microwells may be configured to toggle between “open” and “closed” configurations. For instance, an “open” microwell or set of microwells may be “closed” or “sealed” using a membrane (e.g., semi-permeable membrane), an oil (e.g., fluorinated oil to cover an aqueous solution), or a lid, as described elsewhere herein.

The well may have a volume of less than 1 milliliter (mL). For instance, the well may be configured to hold a volume of at most 1000 microliters (μL), at most 100 μL, at most 10 μL, at most 1 μL, at most 100 nanoliters (nL), at most 10 nL, at most 1 nL, at most 100 picoliters (pL), at most 10 (pL), or less. The well may be configured to hold a volume of about 1000 μL, about 100 μL, about 10 μL, about 1 μL, about 100 nL, about 10 nL, about 1 nL, about 100 pL, about 10 pL, etc. The well may be configured to hold a volume of at least 10 pL, at least 100 pL, at least 1 nL, at least 10 nL, at least 100 nL, at least 1 μL, at least 10 μL, at least 100 μL, at least 1000 μL, or more. The well may be configured to hold a volume in a range of volumes listed herein, for example, from about 5 nL to about 20 nL, from about 1 nL to about 100 nL, from about 500 pL to about 100 μL, etc. The well may be of a plurality of wells that have varying volumes and may be configured to hold a volume appropriate to accommodate any of the partition volumes described herein.

In some instances, a microwell array or plate comprises a single variety of microwells. In some instances, a microwell array or plate comprises a variety of microwells. For instance, the microwell array or plate may comprise one or more types of microwells within a single microwell array or plate. The types of microwells may have different dimensions (e.g., length, width, diameter, depth, cross-sectional area, etc.), shapes (e.g., circular, triangular, square, rectangular, pentagonal, hexagonal, heptagonal, octagonal, nonagonal, decagonal, etc.), aspect ratios, or other physical characteristics. The microwell array or plate may comprise any number of different types of microwells. For example, the microwell array or plate may comprise 1, 2, 3, 4, 5, 6, 7, 8, 9, 10, 20, 30, 40, 50, 60, 70, 80, 90, 100, 200, 300, 400, 500, 600, 700, 800, 900, 1000 or more different types of microwells. A well may have any dimension (e.g., length, width, diameter, depth, cross-sectional area, volume, etc.), shape (e.g., circular, triangular, square, rectangular, pentagonal, hexagonal, heptagonal, octagonal, nonagonal, decagonal, other polygonal, etc.), aspect ratios, or other physical characteristics described herein with respect to any well.

In certain instances, the microwell array or plate comprises different types of microwells that are located adjacent to one another within the array or plate. For instance, a microwell with one set of dimensions may be located adjacent to and in contact with another microwell with a different set of dimensions. Similarly, microwells of different geometries may be placed adjacent to or in contact with one another. The adjacent microwells may be configured to hold different articles; for example, one microwell may be used to contain a cell, cell bead, or other sample (e.g., cellular components, nucleic acid molecules, etc.) while the adjacent microwell may be used to contain a microcapsule, droplet, bead, or other reagent. In some cases, the adjacent microwells may be configured to merge the contents held within, e.g., upon application of a stimulus, or spontaneously, upon contact of the articles in each microwell.

As is described elsewhere herein, a plurality of partitions may be used in the systems, compositions, and methods described herein. For example, any suitable number of partitions (e.g., wells or droplets) can be generated or otherwise provided. For example, in the case when wells are used, at least about 1,000 wells, at least about 5,000 wells, at least about 10,000 wells, at least about 50,000 wells, at least about 100,000 wells, at least about 500,000 wells, at least about 1,000,000 wells, at least about 5,000,000 wells at least about 10,000,000 wells, at least about 50,000,000 wells, at least about 100,000,000 wells, at least about 500,000,000 wells, at least about 1,000,000,000 wells, or more wells can be generated or otherwise provided. Moreover, the plurality of wells may comprise both unoccupied wells (e.g., empty wells) and occupied wells.

A well may comprise any of the reagents described herein, or combinations thereof. These reagents may include, for example, barcode molecules, enzymes, adapters, and combinations thereof. The reagents may be physically separated from a sample (e.g., a cell, cell bead, or cellular components, e.g., proteins, nucleic acid molecules, etc.) that is placed in the well. This physical separation may be accomplished by containing the reagents within, or coupling to, a microcapsule or bead that is placed within a well. The physical separation may also be accomplished by dispensing the reagents in the well and overlaying the reagents with a layer that is, for example, dissolvable, meltable, or permeable prior to introducing the polynucleotide sample into the well. This layer may be, for example, an oil, wax, membrane (e.g., semi-permeable membrane), or the like. The well may be sealed at any point, for example, after addition of the microcapsule or bead, after addition of the reagents, or after addition of either of these components. The sealing of the well may be useful for a variety of purposes, including preventing escape of beads or loaded reagents from the well, permitting select delivery of certain reagents (e.g., via the use of a semi-permeable membrane), for storage of the well prior to or following further processing, etc.

A well may comprise free reagents and/or reagents encapsulated in, or otherwise coupled to or associated with, microcapsules, beads, or droplets. Any of the reagents described in this disclosure may be encapsulated in, or otherwise coupled to, a microcapsule, droplet, or bead, with any chemicals, particles, and elements suitable for sample processing reactions involving biomolecules, such as, but not limited to, nucleic acid molecules and proteins. For example, a bead or droplet used in a sample preparation reaction for DNA sequencing may comprise one or more of the following reagents: enzymes, restriction enzymes (e.g., multiple cutters), ligase, polymerase, fluorophores, oligonucleotide barcodes, adapters, buffers, nucleotides (e.g., dNTPs, ddNTPs) and the like.

Additional examples of reagents include, but are not limited to: buffers, acidic solution, basic solution, temperature-sensitive enzymes, pH-sensitive enzymes, light-sensitive enzymes, metals, metal ions, magnesium chloride, sodium chloride, manganese, aqueous buffer, mild buffer, ionic buffer, inhibitor, enzyme, protein, polynucleotide, antibodies, saccharides, lipid, oil, salt, ion, detergents, ionic detergents, non-ionic detergents, oligonucleotides, nucleotides, deoxyribonucleotide triphosphates (dNTPs), dideoxyribonucleotide triphosphates (ddNTPs), DNA, RNA, peptide polynucleotides, complementary DNA (cDNA), double stranded DNA (dsDNA), single stranded DNA (ssDNA), plasmid DNA, cosmid DNA, chromosomal DNA, genomic DNA, viral DNA, bacterial DNA, mtDNA (mitochondrial DNA), mRNA, rRNA, tRNA, nRNA, siRNA, snRNA, snoRNA, scaRNA, microRNA, dsRNA, ribozyme, riboswitch and viral RNA, polymerase, ligase, restriction enzymes, proteases, nucleases, protease inhibitors, nuclease inhibitors, chelating agents, reducing agents, oxidizing agents, fluorophores, probes, chromophores, dyes, organics, emulsifiers, surfactants, stabilizers, polymers, water, small molecules, pharmaceuticals, radioactive molecules, preservatives, antibiotics, aptamers, and pharmaceutical drug compounds. As described herein, one or more reagents in the well may be used to perform one or more reactions, including but not limited to: cell lysis, cell fixation, permeabilization, nucleic acid reactions, e.g., nucleic acid extension reactions, amplification, reverse transcription, transposase reactions (e.g., tagmentation), etc.

The wells may be provided as a part of a kit. For example, a kit may comprise instructions for use, a microwell array or device, and reagents (e.g., beads). The kit may comprise any useful reagents for performing the processes described herein, e.g., nucleic acid reactions, barcoding of nucleic acid molecules, sample processing (e.g., for cell lysis, fixation, and/or permeabilization).

In some cases, a well comprises a microcapsule, bead, or droplet that comprises a set of reagents that has a similar attribute (e.g., a set of enzymes, a set of minerals, a set of oligonucleotides, a mixture of different barcode molecules, a mixture of identical barcode molecules). In other cases, a microcapsule, bead, or droplet comprises a heterogeneous mixture of reagents. In some cases, the heterogeneous mixture of reagents can comprise all components necessary to perform a reaction. In some cases, such mixture can comprise all components necessary to perform a reaction, except for 1, 2, 3, 4, 5, or more components necessary to perform a reaction. In some cases, such additional components are contained within, or otherwise coupled to, a different microcapsule, droplet, or bead, or within a solution within a partition (e.g., microwell) of the system.

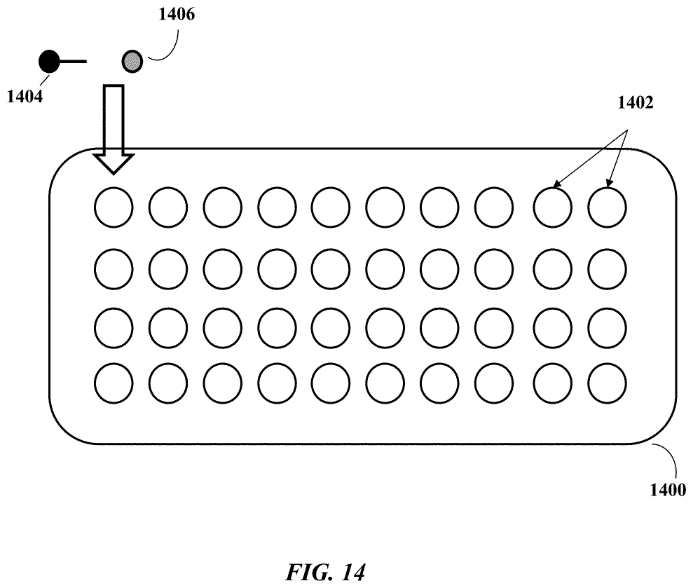

FIG. 14 schematically illustrates an example of a microwell array. The array can be contained within a substrate 1400. The substrate 1400 comprises a plurality of wells 1402. The wells 1002 may be of any size or shape, and the spacing between the wells, the number of wells per substrate, as well as the density of the wells on the substrate 1400 can be modified, depending on the particular application. In one such example application, a sample molecule 1406, which may comprise a cell or cellular components (e.g., nucleic acid molecules) is co-partitioned with a bead 1004, which may comprise a nucleic acid barcode molecule coupled thereto. The wells 1002 may be loaded using gravity or other loading technique (e.g., centrifugation, liquid handler, acoustic loading, optoelectronic, etc.). In some instances, at least one of the wells 1402 contains a single sample molecule 1406 (e.g., cell) and a single bead 1404.

Reagents may be loaded into a well either sequentially or concurrently. In some cases, reagents are introduced to the device either before or after a particular operation. In some cases, reagents (which may be provided, in certain instances, in microcapsules, droplets, or beads) are introduced sequentially such that different reactions or operations occur at different steps. The reagents (or microcapsules, droplets, or beads) may also be loaded at operations interspersed with a reaction or operation step. For example, microcapsules (or droplets or beads) comprising reagents for fragmenting polynucleotides (e.g., restriction enzymes) and/or other enzymes (e.g., transposases, ligases, polymerases, etc.) may be loaded into the well or plurality of wells, followed by loading of microcapsules, droplets, or beads comprising reagents for attaching nucleic acid barcode molecules to a sample nucleic acid molecule. Reagents may be provided concurrently or sequentially with a sample, e.g., a cell or cellular components (e.g., organelles, proteins, nucleic acid molecules, carbohydrates, lipids, etc.). Accordingly, use of wells may be useful in performing multi-step operations or reactions.

As described elsewhere herein, the nucleic acid barcode molecules and other reagents may be contained within a microcapsule, bead, or droplet. These microcapsules, beads, or droplets may be loaded into a partition (e.g., a microwell) before, after, or concurrently with the loading of a cell, such that each cell is contacted with a different microcapsule, bead, or droplet. This technique may be used to attach a unique nucleic acid barcode molecule to nucleic acid molecules obtained from each cell. Alternatively or in addition to, the sample nucleic acid molecules may be attached to a support. For instance, the partition (e.g., microwell) may comprise a bead which has coupled thereto a plurality of nucleic acid barcode molecules. The sample nucleic acid molecules, or derivatives thereof, may couple or attach to the nucleic acid barcode molecules on the support. The resulting barcoded nucleic acid molecules may then be removed from the partition, and in some instances, pooled and sequenced. In such cases, the nucleic acid barcode sequences may be used to trace the origin of the sample nucleic acid molecule. For example, polynucleotides with identical barcodes may be determined to originate from the same cell or partition, while polynucleotides with different barcodes may be determined to originate from different cells or partitions.

The samples or reagents may be loaded in the wells or microwells using a variety of approaches. The samples (e.g., a cell, cell bead, or cellular component) or reagents (as described herein) may be loaded into the well or microwell using an external force, e.g., gravitational force, electrical force, magnetic force, or using mechanisms to drive the sample or reagents into the well, e.g., via pressure-driven flow, centrifugation, optoelectronics, acoustic loading, electrokinetic pumping, vacuum, capillary flow, etc. In certain cases, a fluid handling system may be used to load the samples or reagents into the well. The loading of the samples or reagents may follow a Poissonian distribution or a non-Poissonian distribution, e.g., super Poisson or sub-Poisson. The geometry, spacing between wells, density, and size of the microwells may be modified to accommodate a useful sample or reagent distribution; for instance, the size and spacing of the microwells may be adjusted such that the sample or reagents may be distributed in a super-Poissonian fashion.

In one particular non-limiting example, the microwell array or plate comprises pairs of microwells, in which each pair of microwells is configured to hold a droplet (e.g., comprising a single cell) and a single bead (such as those described herein, which may, in some instances, also be encapsulated in a droplet). The droplet and the bead (or droplet containing the bead) may be loaded simultaneously or sequentially, and the droplet and the bead may be merged, e.g., upon contact of the droplet and the bead, or upon application of a stimulus (e.g., external force, agitation, heat, light, magnetic or electric force, etc.). In some cases, the loading of the droplet and the bead is super-Poissonian. In other examples of pairs of microwells, the wells are configured to hold two droplets comprising different reagents and/or samples, which are merged upon contact or upon application of a stimulus. In such instances, the droplet of one microwell of the pair can comprise reagents that may react with an agent in the droplet of the other microwell of the pair. For instance, one droplet can comprise reagents that are configured to release the nucleic acid barcode molecules of a bead contained in another droplet, located in the adjacent microwell. Upon merging of the droplets, the nucleic acid barcode molecules may be released from the bead into the partition (e.g., the microwell or microwell pair that are in contact), and further processing may be performed (e.g., barcoding, nucleic acid reactions, etc.). In cases where intact or live cells are loaded in the microwells, one of the droplets may comprise lysis reagents for lysing the cell upon droplet merging.

A droplet or microcapsule may be partitioned into a well. The droplets may be selected or subjected to pre-processing prior to loading into a well. For instance, the droplets may comprise cells, and only certain droplets, such as those containing a single cell (or at least one cell), may be selected for use in loading of the wells. Such a pre-selection process may be useful in efficient loading of single cells, such as to obtain a non-Poissonian distribution, or to pre-filter cells for a selected characteristic prior to further partitioning in the wells. Additionally, the technique may be useful in obtaining or preventing cell doublet or multiplet formation prior to or during loading of the microwell.

In some instances, the wells can comprise nucleic acid barcode molecules attached thereto. The nucleic acid barcode molecules may be attached to a surface of the well (e.g., a wall of the well). The nucleic acid barcode molecule (e.g., a partition barcode sequence) of one well may differ from the nucleic acid barcode molecule of another well, which can permit identification of the contents contained with a single partition or well. In some cases, the nucleic acid barcode molecule can comprise a spatial barcode sequence that can identify a spatial coordinate of a well, such as within the well array or well plate. In some cases, the nucleic acid barcode molecule can comprise a unique molecular identifier for individual molecule identification. In some instances, the nucleic acid barcode molecules may be configured to attach to or capture a nucleic acid molecule within a sample or cell distributed in the well. For example, the nucleic acid barcode molecules may comprise a capture sequence that may be used to capture or hybridize to a nucleic acid molecule (e.g., RNA, DNA) within the sample. In some instances, the nucleic acid barcode molecules may be releasable from the microwell. For instance, the nucleic acid barcode molecules may comprise a chemical cross-linker which may be cleaved upon application of a stimulus (e.g., photo-, magnetic, chemical, biological, stimulus). The released nucleic acid barcode molecules, which may be hybridized or configured to hybridize to a sample nucleic acid molecule, may be collected and pooled for further processing, which can include nucleic acid processing (e.g., amplification, extension, reverse transcription, etc.) and/or characterization (e.g., sequencing). In such cases, the unique partition barcode sequences may be used to identify the cell or partition from which a nucleic acid molecule originated.

Characterization of samples within a well may be performed. Such characterization can include, in non-limiting examples, imaging of the sample (e.g., cell, cell bead, or cellular components) or derivatives thereof. Characterization techniques such as microscopy or imaging may be useful in measuring sample profiles in fixed spatial locations. For instance, when cells are partitioned, optionally with beads, imaging of each microwell and the contents contained therein may provide useful information on cell doublet formation (e.g., frequency, spatial locations, etc.), cell-bead pair efficiency, cell viability, cell size, cell morphology, expression level of a biomarker (e.g., a surface marker, a fluorescently labeled molecule therein, etc.), cell or bead loading rate, number of cell-bead pairs, etc. In some instances, imaging may be used to characterize live cells in the wells, including, but not limited to: dynamic live-cell tracking, cell-cell interactions (when two or more cells are co-partitioned), cell proliferation, etc. Alternatively or in addition to, imaging may be used to characterize a quantity of amplification products in the well.

In operation, a well may be loaded with a sample and reagents, simultaneously or sequentially. When cells or cell beads are loaded, the well may be subjected to washing, e.g., to remove excess cells from the well, microwell array, or plate. Similarly, washing may be performed to remove excess beads or other reagents from the well, microwell array, or plate. In the instances where live cells are used, the cells may be lysed in the individual partitions to release the intracellular components or cellular analytes. Alternatively, the cells may be fixed or permeabilized in the individual partitions. The intracellular components or cellular analytes may couple to a support, e.g., on a surface of the microwell, on a solid support (e.g., bead), or they may be collected for further downstream processing. For instance, after cell lysis, the intracellular components or cellular analytes may be transferred to individual droplets or other partitions for barcoding. Alternatively, or in addition to, the intracellular components or cellular analytes (e.g., nucleic acid molecules) may couple to a bead comprising a nucleic acid barcode molecule; subsequently, the bead may be collected and further processed, e.g., subjected to nucleic acid reaction such as reverse transcription, amplification, or extension, and the nucleic acid molecules thereon may be further characterized, e.g., via sequencing. Alternatively, or in addition to, the intracellular components or cellular analytes may be barcoded in the well (e.g., using a bead comprising nucleic acid barcode molecules that are releasable or on a surface of the microwell comprising nucleic acid barcode molecules). The barcoded nucleic acid molecules or analytes may be further processed in the well, or the barcoded nucleic acid molecules or analytes may be collected from the individual partitions and subjected to further processing outside the partition. Further processing can include nucleic acid processing (e.g., performing an amplification, extension) or characterization (e.g., fluorescence monitoring of amplified molecules, sequencing). At any convenient or useful step, the well (or microwell array or plate) may be sealed (e.g., using an oil, membrane, wax, etc.), which enables storage of the assay or selective introduction of additional reagents.

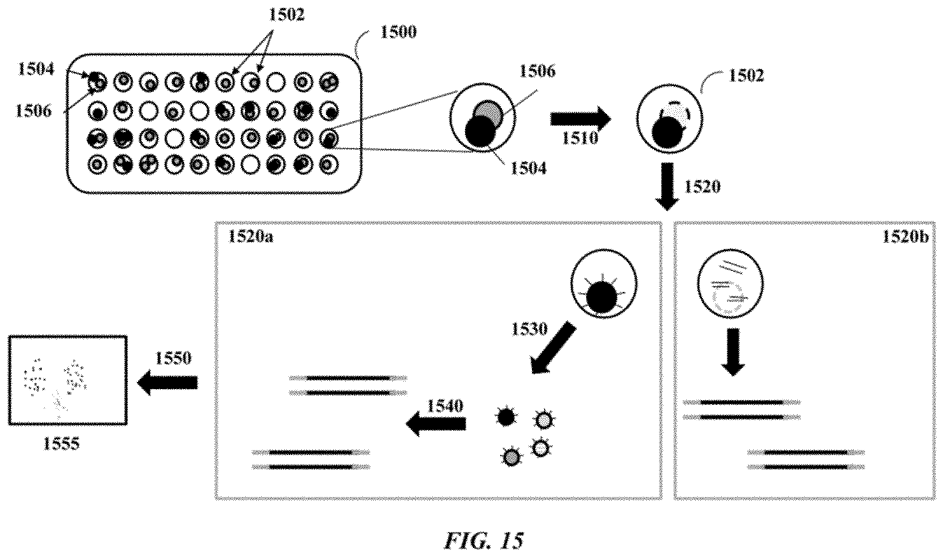

FIG. 15 schematically shows an example workflow for processing nucleic acid molecules within a sample. A substrate 1500 comprising a plurality of microwells 1502 may be provided. A sample 1506 which may comprise a cell, cell bead, cellular components or analytes (e.g., proteins and/or nucleic acid molecules) can be co-partitioned, in a plurality of microwells 1502, with a plurality of beads 1504 comprising nucleic acid barcode molecules. During process 1510, the sample 1506 may be processed within the partition. For instance, in the case of live cells, the cell may be subjected to conditions sufficient to lyse the cells and release the analytes contained therein. In process 1520, the bead 1504 may be further processed. By way of example, processes 1520a and 1520b schematically illustrate different workflows, depending on the properties of the bead 1504.

In 1520a, the bead comprises nucleic acid barcode molecules that are attached thereto, and sample nucleic acid molecules (e.g., RNA, DNA) may attach, e.g., via hybridization of ligation, to the nucleic acid barcode molecules. Such attachment may occur on the bead. In process 1530, the beads 1504 from multiple wells 1502 may be collected and pooled. Further processing may be performed in process 1540. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 1550, further characterization, such as sequencing may be performed to generate sequencing reads. The sequencing reads may yield information on individual cells or populations of cells, which may be represented visually or graphically, e.g., in a plot 1555.

In 1520b, the bead comprises nucleic acid barcode molecules that are releasably attached thereto, as described below. The bead may degrade or otherwise release the nucleic acid barcode molecules into the well 1502; the nucleic acid barcode molecules may then be used to barcode nucleic acid molecules within the well 1502. Further processing may be performed either inside the partition or outside the partition. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 1550, further characterization, such as sequencing may be performed to generate sequencing reads. The sequencing reads may yield information on individual cells or populations of cells, which may be represented visually or graphically, e.g., in a plot 1555.

### Compositions

Further provided herein are also compositions, comprising: a plurality of cells comprising a plurality of nucleic acid barcode molecules coupled thereto, wherein a cell of said plurality of cells comprises a nucleic acid barcode molecule of said plurality of nucleic acid barcode molecules coupled to a surface of said cell, wherein said nucleic acid barcode molecule comprises (i) a barcode sequence unique to said cell among said plurality of cells, and (ii) a capture sequence configured to capture an analyte. In some instances, said plurality of cells are provided in bulk solution. In some instances, said plurality of cells are provided in a plurality of partitions. In some instances, said plurality of partitions are a plurality of droplets.

In some instances, said plurality of partitions are a plurality of wells. In some instances, said plurality of partitions are microwells or nanowells. In some instances, said plurality of partitions are nanowells in a nanowell array. In some instances, plurality of partitions are microwells from a 96-well plate or a 384-well plate.

In some instances, a partition of said plurality of partitions comprises said cell. In some instances, said nucleic acid barcode molecule is coupled to said surface of said cell via a cell coupling agent. In some instances, said cell coupling agent comprises a peptide or polypeptide. In some instances, said peptide or polypeptide is coupled to an antigen on said surface of said cell. In some instances, said peptide or polypeptide is coupled to a carbohydrate group on a cell membrane of said cell. [In some instances, said cell coupling agent comprises a lipid molecule, wherein said lipid molecule is embedded into a cell membrane of said cell. In some instances, said cell coupling agent comprises a disulfide bond. In some instances, said capture sequence comprises a poly-T sequence. In some instances, said capture sequence comprises a template switching oligonucleotide sequence. In some instances, said capture sequence comprises a poly-G sequence.

### Systems

Also provided herein are systems comprising: a plurality of partitions comprising a plurality of cells, wherein said plurality of cells comprises a plurality of nucleic acid barcode molecules coupled thereto, wherein a partition of said plurality of partitions comprises (i) a cell of said plurality of cells, wherein said cell comprises a nucleic acid barcode molecule of said plurality of nucleic acid barcode molecules coupled to a surface of said cell, wherein said barcode molecule comprises a barcode sequence unique to said cell among said plurality of cells, (ii) a nucleic acid molecule comprising a capture sequence configured to capture an analyte, and (iii) reagents configured to couple said nucleic acid molecule to said nucleic acid barcode molecule to generate a composite barcode molecule comprising said barcode sequence and said capture sequence.

In some instances, said reagents comprise a splint molecule configured to couple to each of said nucleic acid barcode molecule and said nucleic acid molecule. In some instances, said plurality of partitions are a plurality of droplets. In some instances, the plurality of partitions are a plurality of wells. In some instances, partition is a microwell or a nanowell. In some instances, said partition is said nanowell, wherein said nanowell is from a nanowell array. In some instances, the microwell is from a 96-well plate or a 384-well plate. In some instances, said partition comprises more than one cell.

In some instances, said nucleic acid barcode molecule is coupled to said surface of said cell via a cell coupling agent. In some instances, said cell coupling agent comprises a peptide or polypeptide. In some instances, said peptide or polypeptide is coupled to an antigen on said surface of said cell. In some instances, said peptide or polypeptide is coupled to a carbohydrate group on a cell membrane of said cell. In some instances, said cell coupling agent comprises a lipid molecule, wherein said lipid molecule is embedded into a cell membrane of said cell. In some instances, said cell coupling agent comprises a disulfide bond. In some instances, said capture sequence comprises a poly-T sequence. In some instances, said capture sequence comprises a template switching oligonucleotide sequence. In some instances, said capture sequence comprises a poly-G sequence.

### Beads

A partition may comprise one or more unique identifiers, such as barcodes. Barcodes may be previously, subsequently or concurrently delivered to the partitions that hold the compartmentalized or partitioned biological particle. For example, barcodes may be injected into droplets previous to, subsequent to, or concurrently with droplet generation. The delivery of the barcodes to a particular partition allows for the later attribution of the characteristics of the individual biological particle to the particular partition. Barcodes may be delivered, for example on a nucleic acid molecule (e.g., an oligonucleotide), to a partition via any suitable mechanism. Barcoded nucleic acid molecules can be delivered to a partition via a microcapsule. A microcapsule, in some instances, can comprise a bead. Beads are described in further detail below.

In some cases, barcoded nucleic acid molecules can be initially associated with the microcapsule and then released from the microcapsule. Release of the barcoded nucleic acid molecules can be passive (e.g., by diffusion out of the microcapsule). In addition or alternatively, release from the microcapsule can be upon application of a stimulus which allows the barcoded nucleic acid nucleic acid molecules to dissociate or to be released from the microcapsule. Such stimulus may disrupt the microcapsule, an interaction that couples the barcoded nucleic acid molecules to or within the microcapsule, or both. Such stimulus can include, for example, a thermal stimulus, photo-stimulus, chemical stimulus (e.g., change in pH or use of a reducing agent(s)), a mechanical stimulus, a radiation stimulus; a biological stimulus (e.g., enzyme), or any combination thereof.

FIG. 2 shows an example of a microfluidic channel structure 200 for delivering barcode carrying beads to droplets. The channel structure 200 can include channel segments 201, 202, 204, 206 and 208 communicating at a channel junction 210. In operation, the channel segment 201 may transport an aqueous fluid 212 that includes a plurality of beads 214 (e.g., with nucleic acid molecules, oligonucleotides, molecular tags) along the channel segment 201 into junction 210. The plurality of beads 214 may be sourced from a suspension of beads. For example, the channel segment 201 may be connected to a reservoir comprising an aqueous suspension of beads 214. The channel segment 202 may transport the aqueous fluid 212 that includes a plurality of biological particles 216 along the channel segment 202 into junction 210. The plurality of biological particles 216 may be sourced from a suspension of biological particles. For example, the channel segment 202 may be connected to a reservoir comprising an aqueous suspension of biological particles 216. In some instances, the aqueous fluid 212 in either the first channel segment 201 or the second channel segment 202, or in both segments, can include one or more reagents, as further described below. A second fluid 218 that is immiscible with the aqueous fluid 212 (e.g., oil) can be delivered to the junction 210 from each of channel segments 204 and 206. Upon meeting of the aqueous fluid 212 from each of channel segments 201 and 202 and the second fluid 218 from each of channel segments 204 and 206 at the channel junction 210, the aqueous fluid 212 can be partitioned as discrete droplets 220 in the second fluid 218 and flow away from the junction 210 along channel segment 208. The channel segment 208 may deliver the discrete droplets to an outlet reservoir fluidly coupled to the channel segment 208, where they may be harvested.

As an alternative, the channel segments 201 and 202 may meet at another junction upstream of the junction 210. At such junction, beads and biological particles may form a mixture that is directed along another channel to the junction 210 to yield droplets 220. The mixture may provide the beads and biological particles in an alternating fashion, such that, for example, a droplet comprises a single bead and a single biological particle.

Beads, biological particles and droplets may flow along channels at substantially regular flow profiles (e.g., at regular flow rates). Such regular flow profiles may permit a droplet to include a single bead and a single biological particle. Such regular flow profiles may permit the droplets to have an occupancy (e.g., droplets having beads and biological particles) greater than 5%, 10%, 20%, 30%, 40%, 50%, 60%, 70%, 80%, 90%, or 95%. Such regular flow profiles and devices that may be used to provide such regular flow profiles are provided in, for example, U.S. Patent Publication No. 2015/0292988, which is entirely incorporated herein by reference.

The second fluid 218 can comprise an oil, such as a fluorinated oil, that includes a fluorosurfactant for stabilizing the resulting droplets, for example, inhibiting subsequent coalescence of the resulting droplets 220.

A discrete droplet that is generated may include an individual biological particle 216. A discrete droplet that is generated may include a barcode or other reagent carrying bead 214. A discrete droplet generated may include both an individual biological particle and a barcode carrying bead, such as droplets 220. In some instances, a discrete droplet may include more than one individual biological particle or no biological particle. In some instances, a discrete droplet may include more than one bead or no bead. A discrete droplet may be unoccupied (e.g., no beads, no biological particles).

Beneficially, a discrete droplet partitioning a biological particle and a barcode carrying bead may effectively allow the attribution of the barcode to intracellular analytes of the biological particle within the partition. The contents of a partition may remain discrete from the contents of other partitions.

As will be appreciated, the channel segments described herein may be coupled to any of a variety of different fluid sources or receiving components, including reservoirs, tubing, manifolds, or fluidic components of other systems. As will be appreciated, the microfluidic channel structure 200 may have other geometries. For example, a microfluidic channel structure can have more than one channel junctions. For example, a microfluidic channel structure can have 2, 3, 4, or 5 channel segments each carrying beads that meet at a channel junction. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

A bead may be porous, non-porous, solid, semi-solid, semi-fluidic, fluidic, and/or a combination thereof In some instances, a bead may be dissolvable, disruptable, and/or degradable. In some cases, a bead may not be degradable. In some cases, the bead may be a gel bead. A gel bead may be a hydrogel bead. A gel bead may be formed from molecular precursors, such as a polymeric or monomeric species. A semi-solid bead may be a liposomal bead. Solid beads may comprise metals including iron oxide, gold, and silver. In some cases, the bead may be a silica bead. In some cases, the bead can be rigid. In other cases, the bead may be flexible and/or compressible.

A bead may be of any suitable shape. Examples of bead shapes include, but are not limited to, spherical, non-spherical, oval, oblong, amorphous, circular, cylindrical, and variations thereof

Beads may be of uniform size or heterogeneous size. In some cases, the diameter of a bead may be at least about 10 nanometers (nm), 100 nm, 500 nm, 1 micrometer (μm), 5μm, 10 μm, 20 μm, 30 μm, 40 μm, 50 μm, 60 μm, 70 μm, 80 μm, 90 μm, 100 μm, 250 μm, 500 μm, 1 mm, or greater. In some cases, a bead may have a diameter of less than about 10 nm, 100 nm, 500 nm, 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a bead may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In certain aspects, beads can be provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In particular, the beads described herein may have size distributions that have a coefficient of variation in their cross-sectional dimensions of less than 50%, less than 40%, less than 30%, less than 20%, and in some cases less than 15%, less than 10%, less than 5%, or less.

A bead may comprise natural and/or synthetic materials. For example, a bead can comprise a natural polymer, a synthetic polymer or both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, Corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads may also be formed from materials other than polymers, including lipids, micelles, ceramics, glass-ceramics, material composites, metals, other inorganic materials, and others.

In some instances, the bead may contain molecular precursors (e.g., monomers or polymers), which may form a polymer network via polymerization of the molecular precursors. In some cases, a precursor may be an already polymerized species capable of undergoing further polymerization via, for example, a chemical cross-linkage. In some cases, a precursor can comprise one or more of an acrylamide or a methacrylamide monomer, oligomer, or polymer. In some cases, the bead may comprise prepolymers, which are oligomers capable of further polymerization. For example, polyurethane beads may be prepared using prepolymers. In some cases, the bead may contain individual polymers that may be further polymerized together. In some cases, beads may be generated via polymerization of different precursors, such that they comprise mixed polymers, co-polymers, and/or block co-polymers. In some cases, the bead may comprise covalent or ionic bonds between polymeric precursors (e.g., monomers, oligomers, linear polymers), nucleic acid molecules (e.g., oligonucleotides), primers, and other entities. In some cases, the covalent bonds can be carbon-carbon bonds, thioether bonds, or carbon-heteroatom bonds.

Cross-linking may be permanent or reversible, depending upon the particular cross-linker used. Reversible cross-linking may allow for the polymer to linearize or dissociate under appropriate conditions. In some cases, reversible cross-linking may also allow for reversible attachment of a material bound to the surface of a bead. In some cases, a cross-linker may form disulfide linkages. In some cases, the chemical cross-linker forming disulfide linkages may be cystamine or a modified cystamine.

In some cases, disulfide linkages can be formed between molecular precursor units (e.g., monomers, oligomers, or linear polymers) or precursors incorporated into a bead and nucleic acid molecules (e.g., oligonucleotides). Cystamine (including modified cystamines), for example, is an organic agent comprising a disulfide bond that may be used as a crosslinker agent between individual monomeric or polymeric precursors of a bead. Polyacrylamide may be polymerized in the presence of cystamine or a species comprising cystamine (e.g., a modified cystamine) to generate polyacrylamide gel beads comprising disulfide linkages (e.g., chemically degradable beads comprising chemically-reducible cross-linkers). The disulfide linkages may permit the bead to be degraded (or dissolved) upon exposure of the bead to a reducing agent.

In some cases, chitosan, a linear polysaccharide polymer, may be crosslinked with glutaraldehyde via hydrophilic chains to form a bead. Crosslinking of chitosan polymers may be achieved by chemical reactions that are initiated by heat, pressure, change in pH, and/or radiation.

In some cases, a bead may comprise an acrydite moiety, which in certain aspects may be used to attach one or more nucleic acid molecules (e.g., barcode sequence, barcoded nucleic acid molecule, barcoded oligonucleotide, primer, or other oligonucleotide) to the bead. In some cases, an acrydite moiety can refer to an acrydite analogue generated from the reaction of acrydite with one or more species, such as, the reaction of acrydite with other monomers and cross-linkers during a polymerization reaction. Acrydite moieties may be modified to form chemical bonds with a species to be attached, such as a nucleic acid molecule (e.g., barcode sequence, barcoded nucleic acid molecule, barcoded oligonucleotide, primer, or other oligonucleotide). Acrydite moieties may be modified with thiol groups capable of forming a disulfide bond or may be modified with groups already comprising a disulfide bond. The thiol or disulfide (via disulfide exchange) may be used as an anchor point for a species to be attached or another part of the acrydite moiety may be used for attachment. In some cases, attachment can be reversible, such that when the disulfide bond is broken (e.g., in the presence of a reducing agent), the attached species is released from the bead. In other cases, an acrydite moiety can comprise a reactive hydroxyl group that may be used for attachment.

Functionalization of beads for attachment of nucleic acid molecules (e.g., oligonucleotides) may be achieved through a wide range of different approaches, including activation of chemical groups within a polymer, incorporation of active or activatable functional groups in the polymer structure, or attachment at the pre-polymer or monomer stage in bead production.

For example, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule (e.g., oligonucleotide), which may include a priming sequence (e.g., a primer for amplifying target nucleic acids, random primer, primer sequence for messenger RNA) and/or one or more barcode sequences. The one more barcode sequences may include sequences that are the same for all nucleic acid molecules coupled to a given bead and/or sequences that are different across all nucleic acid molecules coupled to the given bead. The nucleic acid molecule may be incorporated into the bead.

In some cases, the nucleic acid molecule can comprise a functional sequence, for example, for attachment to a sequencing flow cell, such as, for example, a P5 sequence for Illumina® sequencing. In some cases, the nucleic acid molecule or derivative thereof (e.g., oligonucleotide or polynucleotide generated from the nucleic acid molecule) can comprise another functional sequence, such as, for example, a P7 sequence for attachment to a sequencing flow cell for Illumina sequencing. In some cases, the nucleic acid molecule can comprise a barcode sequence. In some cases, the primer can further comprise a unique molecular identifier (UMI). In some cases, the primer can comprise an R1 primer sequence for Illumina sequencing. In some cases, the primer can comprise an R2 primer sequence for Illumina sequencing. Examples of such nucleic acid molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is entirely incorporated herein by reference.

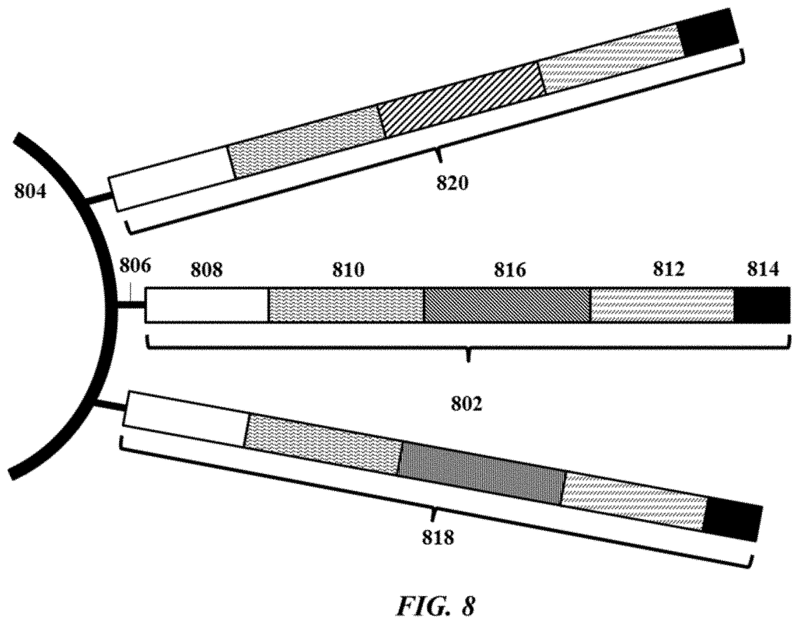

FIG. 8 illustrates an example of a barcode carrying bead. A nucleic acid molecule 802, such as an oligonucleotide, can be coupled to a bead 804 by a releasable linkage 806, such as, for example, a disulfide linker. The same bead 804 may be coupled (e.g., via releasable linkage) to one or more other nucleic acid molecules 818, 820. The nucleic acid molecule 802 may be or comprise a barcode. As noted elsewhere herein, the structure of the barcode may comprise a number of sequence elements. The nucleic acid molecule 802 may comprise a functional sequence 808 that may be used in subsequent processing. For example, the functional sequence 808 may include one or more of a sequencer specific flow cell attachment sequence (e.g., a P5 sequence for Illumina® sequencing systems) and a sequencing primer sequence (e.g., a R1 primer for Illumina® sequencing systems). The nucleic acid molecule 802 may comprise a barcode sequence 810 for use in barcoding the sample (e.g., DNA, RNA, protein, etc.). In some cases, the barcode sequence 810 can be bead-specific such that the barcode sequence 810 is common to all nucleic acid molecules (e.g., including nucleic acid molecule 802) coupled to the same bead 804. Alternatively or in addition, the barcode sequence 810 can be partition-specific such that the barcode sequence 810 is common to all nucleic acid molecules coupled to one or more beads that are partitioned into the same partition. The nucleic acid molecule 802 may comprise a specific priming sequence 812, such as an mRNA specific priming sequence (e.g., poly-T sequence), a targeted priming sequence, and/or a random priming sequence. The nucleic acid molecule 802 may comprise an anchoring sequence 814 to ensure that the specific priming sequence 812 hybridizes at the sequence end (e.g., of the mRNA). For example, the anchoring sequence 814 can include a random short sequence of nucleotides, such as a 1-mer, 2-mer, 3-mer or longer sequence, which can ensure that a poly-T segment is more likely to hybridize at the sequence end of the poly-A tail of the mRNA.

The nucleic acid molecule 802 may comprise a unique molecular identifying sequence 816 (e.g., unique molecular identifier (UMI)). In some cases, the unique molecular identifying sequence 816 may comprise from about 5 to about 8 nucleotides. Alternatively, the unique molecular identifying sequence 816 may compress less than about 5 or more than about 8 nucleotides. The unique molecular identifying sequence 816 may be a unique sequence that varies across individual nucleic acid molecules (e.g., 802, 818, 820, etc.) coupled to a single bead (e.g., bead 804). In some cases, the unique molecular identifying sequence 816 may be a random sequence (e.g., such as a random N-mer sequence). For example, the UMI may provide a unique identifier of the starting mRNA molecule that was captured, in order to allow quantitation of the number of original expressed RNA. As will be appreciated, although FIG. 8 shows three nucleic acid molecules 802, 818, 820 coupled to the surface of the bead 804, an individual bead may be coupled to any number of individual nucleic acid molecules, for example, from one to tens to hundreds of thousands or even millions of individual nucleic acid molecules. The respective barcodes for the individual nucleic acid molecules can comprise both common sequence segments or relatively common sequence segments (e.g., 808, 810, 812, etc.) and variable or unique sequence segments (e.g., 816) between different individual nucleic acid molecules coupled to the same bead.

In operation, a biological particle (e.g., cell, DNA, RNA, etc.) can be co-partitioned along with a barcode bearing bead 804. The barcoded nucleic acid molecules 802, 818, 820 can be released from the bead 804 in the partition. By way of example, in the context of analyzing sample RNA, the poly-T segment (e.g., 812) of one of the released nucleic acid molecules (e.g., 802) can hybridize to the poly-A tail of a mRNA molecule. Reverse transcription may result in a cDNA transcript of the mRNA, but which transcript includes each of the sequence segments 808, 810, 816 of the nucleic acid molecule 802. Because the nucleic acid molecule 802 comprises an anchoring sequence 814, it will more likely hybridize to and prime reverse transcription at the sequence end of the poly-A tail of the mRNA. Within any given partition, all of the cDNA transcripts of the individual mRNA molecules may include a common barcode sequence segment 810. However, the transcripts made from the different mRNA molecules within a given partition may vary at the unique molecular identifying sequence 812 segment (e.g., UMI segment). Beneficially, even following any subsequent amplification of the contents of a given partition, the number of different UMIs can be indicative of the quantity of mRNA originating from a given partition, and thus from the biological particle (e.g., cell). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences may also be used in priming the reverse transcription reaction. Likewise, although described as releasing the barcoded oligonucleotides into the partition, in some cases, the nucleic acid molecules bound to the bead (e.g., gel bead) may be used to hybridize and capture the mRNA on the solid phase of the bead, for example, in order to facilitate the separation of the RNA from other cell contents.

In some cases, precursors comprising a functional group that is reactive or capable of being activated such that it becomes reactive can be polymerized with other precursors to generate gel beads comprising the activated or activatable functional group. The functional group may then be used to attach additional species (e.g., disulfide linkers, primers, other oligonucleotides, etc.) to the gel beads. For example, some precursors comprising a carboxylic acid (COOH) group can co-polymerize with other precursors to form a gel bead that also comprises a COOH functional group. In some cases, acrylic acid (a species comprising free COOH groups), acrylamide, and bis(acryloyl)cystamine can be co-polymerized together to generate a gel bead comprising free COOH groups. The COOH groups of the gel bead can be activated (e.g., via 1-Ethyl-3-(3-dimethylaminopropyl)carbodiimide (EDC) and N-Hydroxysuccinimide (NHS) or 4-(4,6-Dimethoxy-1,3,5-triazin-2-yl)-4-methylmorpholinium chloride (DMTMM)) such that they are reactive (e.g., reactive to amine functional groups where EDC/NHS or DMTMM are used for activation). The activated COOH groups can then react with an appropriate species (e.g., a species comprising an amine functional group where the carboxylic acid groups are activated to be reactive with an amine functional group) comprising a moiety to be linked to the bead.

Beads comprising disulfide linkages in their polymeric network may be functionalized with additional species via reduction of some of the disulfide linkages to free thiols. The disulfide linkages may be reduced via, for example, the action of a reducing agent (e.g., DTT, TCEP, etc.) to generate free thiol groups, without dissolution of the bead. Free thiols of the beads can then react with free thiols of a species or a species comprising another disulfide bond (e.g., via thiol-disulfide exchange) such that the species can be linked to the beads (e.g., via a generated disulfide bond). In some cases, free thiols of the beads may react with any other suitable group. For example, free thiols of the beads may react with species comprising an acrydite moiety. The free thiol groups of the beads can react with the acrydite via Michael addition chemistry, such that the species comprising the acrydite is linked to the bead. In some cases, uncontrolled reactions can be prevented by inclusion of a thiol capping agent such as N-ethylmalieamide or iodoacetate.

Activation of disulfide linkages within a bead can be controlled such that only a small number of disulfide linkages are activated. Control may be exerted, for example, by controlling the concentration of a reducing agent used to generate free thiol groups and/or concentration of reagents used to form disulfide bonds in bead polymerization. In some cases, a low concentration (e.g., molecules of reducing agent:gel bead ratios of less than or equal to about 1:100,000,000,000, less than or equal to about 1:10,000,000,000, less than or equal to about 1:1,000,000,000, less than or equal to about 1:100,000,000, less than or equal to about 1:10,000,000, less than or equal to about 1:1,000,000, less than or equal to about 1:100,000, less than or equal to about 1:10,000) of reducing agent may be used for reduction. Controlling the number of disulfide linkages that are reduced to free thiols may be useful in ensuring bead structural integrity during functionalization. In some cases, optically-active agents, such as fluorescent dyes may be coupled to beads via free thiol groups of the beads and used to quantify the number of free thiols present in a bead and/or track a bead.

In some cases, addition of moieties to a gel bead after gel bead formation may be advantageous. For example, addition of an oligonucleotide (e.g., barcoded oligonucleotide) after gel bead formation may avoid loss of the species during chain transfer termination that can occur during polymerization. Moreover, smaller precursors (e.g., monomers or cross linkers that do not comprise side chain groups and linked moieties) may be used for polymerization and can be minimally hindered from growing chain ends due to viscous effects. In some cases, functionalization after gel bead synthesis can minimize exposure of species (e.g., oligonucleotides) to be loaded with potentially damaging agents (e.g., free radicals) and/or chemical environments. In some cases, the generated gel may possess an upper critical solution temperature (UCST) that can permit temperature driven swelling and collapse of a bead. Such functionality may aid in oligonucleotide (e.g., a primer) infiltration into the bead during subsequent functionalization of the bead with the oligonucleotide. Post-production functionalization may also be useful in controlling loading ratios of species in beads, such that, for example, the variability in loading ratio is minimized. Species loading may also be performed in a batch process such that a plurality of beads can be functionalized with the species in a single batch.

A bead injected or otherwise introduced into a partition may comprise releasably, cleavably, or reversibly attached barcodes. A bead injected or otherwise introduced into a partition may comprise activatable barcodes. A bead injected or otherwise introduced into a partition may be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage may be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In addition to, or as an alternative to the cleavable linkages between the beads and the associated molecules, such as barcode containing nucleic acid molecules (e.g., barcoded oligonucleotides), the beads may be degradable, disruptable, or dissolvable spontaneously or upon exposure to one or more stimuli (e.g., temperature changes, pH changes, exposure to particular chemical species or phase, exposure to light, reducing agent, etc.). In some cases, a bead may be dissolvable, such that material components of the beads are solubilized when exposed to a particular chemical species or an environmental change, such as a change temperature or a change in pH. In some cases, a gel bead can be degraded or dissolved at elevated temperature and/or in basic conditions. In some cases, a bead may be thermally degradable such that when the bead is exposed to an appropriate change in temperature (e.g., heat), the bead degrades. Degradation or dissolution of a bead bound to a species (e.g., a nucleic acid molecule, e.g., barcoded oligonucleotide) may result in release of the species from the bead.

As will be appreciated from the above disclosure, the degradation of a bead may refer to the disassociation of a bound or entrained species from a bead, both with and without structurally degrading the physical bead itself. For example, the degradation of the bead may involve cleavage of a cleavable linkage via one or more species and/or methods described elsewhere herein. In another example, entrained species may be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead may cause a bead to better retain an entrained species due to pore size contraction.

A degradable bead may be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid molecules) may interact with other reagents contained in the partition. For example, a polyacrylamide bead comprising cystamine and linked, via a disulfide bond, to a barcode sequence, may be combined with a reducing agent within a droplet of a water-in-oil emulsion. Within the droplet, the reducing agent can break the various disulfide bonds, resulting in bead degradation and release of the barcode sequence into the aqueous, inner environment of the droplet. In another example, heating of a droplet comprising a bead-bound barcode sequence in basic solution may also result in bead degradation and release of the attached barcode sequence into the aqueous, inner environment of the droplet.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing nucleic acid molecule (e.g., oligonucleotide) bearing beads.

In some cases, beads can be non-covalently loaded with one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads may be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads may be accomplished by various swelling methods. The de-swelling of the beads may be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads may be accomplished by various de-swelling methods. Transferring the beads may cause pores in the bead to shrink. The shrinking may then hinder reagents within the beads from diffusing out of the interiors of the beads. The hindrance may be due to steric interactions between the reagents and the interiors of the beads. The transfer may be accomplished microfluidically. For instance, the transfer may be achieved by moving the beads from one co-flowing solvent stream to a different co-flowing solvent stream. The swellability and/or pore size of the beads may be adjusted by changing the polymer composition of the bead.

In some cases, an acrydite moiety linked to a precursor, another species linked to a precursor, or a precursor itself can comprise a labile bond, such as chemically, thermally, or photo-sensitive bond e.g., disulfide bond, UV sensitive bond, or the like. Once acrydite moieties or other moieties comprising a labile bond are incorporated into a bead, the bead may also comprise the labile bond. The labile bond may be, for example, useful in reversibly linking (e.g., covalently linking) species (e.g., barcodes, primers, etc.) to a bead. In some cases, a thermally labile bond may include a nucleic acid hybridization based attachment, e.g., where an oligonucleotide is hybridized to a complementary sequence that is attached to the bead, such that thermal melting of the hybrid releases the oligonucleotide, e.g., a barcode containing sequence, from the bead or microcapsule.

The addition of multiple types of labile bonds to a gel bead may result in the generation of a bead capable of responding to varied stimuli. Each type of labile bond may be sensitive to an associated stimulus (e.g., chemical stimulus, light, temperature, enzymatic, etc.) such that release of species attached to a bead via each labile bond may be controlled by the application of the appropriate stimulus. Such functionality may be useful in controlled release of species from a gel bead. In some cases, another species comprising a labile bond may be linked to a gel bead after gel bead formation via, for example, an activated functional group of the gel bead as described above. As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

The barcodes that are releasable as described herein may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In addition to thermally cleavable bonds, disulfide bonds and UV sensitive bonds, other non-limiting examples of labile bonds that may be coupled to a precursor or bead include an ester linkage (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)). A bond may be cleavable via other nucleic acid molecule targeting enzymes, such as restriction enzymes (e.g., restriction endonucleases), as described further below.

Species may be encapsulated in beads during bead generation (e.g., during polymerization of precursors). Such species may or may not participate in polymerization. Such species may be entered into polymerization reaction mixtures such that generated beads comprise the species upon bead formation. In some cases, such species may be added to the gel beads after formation. Such species may include, for example, nucleic acid molecules (e.g., oligonucleotides), reagents for a nucleic acid amplification reaction (e.g., primers, polymerases, dNTPs, co-factors (e.g., ionic co-factors), buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species may include one or more enzymes described herein, including without limitation, polymerase, reverse transcriptase, restriction enzymes (e.g., endonuclease), transposase, ligase, proteinase K, DNAse, etc. Such species may include one or more reagents described elsewhere herein (e.g., lysis agents, inhibitors, inactivating agents, chelating agents, stimulus). Trapping of such species may be controlled by the polymer network density generated during polymerization of precursors, control of ionic charge within the gel bead (e.g., via ionic species linked to polymerized species), or by the release of other species. Encapsulated species may be released from a bead upon bead degradation and/or by application of a stimulus capable of releasing the species from the bead. Alternatively or in addition, species may be partitioned in a partition (e.g., droplet) during or subsequent to partition formation. Such species may include, without limitation, the abovementioned species that may also be encapsulated in a bead.

A degradable bead may comprise one or more species with a labile bond such that, when the bead/species is exposed to the appropriate stimuli, the bond is broken and the bead degrades. The labile bond may be a chemical bond (e.g., covalent bond, ionic bond) or may be another type of physical interaction (e.g., van der Waals interactions, dipole-dipole interactions, etc.). In some cases, a crosslinker used to generate a bead may comprise a labile bond. Upon exposure to the appropriate conditions, the labile bond can be broken and the bead degraded. For example, upon exposure of a polyacrylamide gel bead comprising cystamine crosslinkers to a reducing agent, the disulfide bonds of the cystamine can be broken and the bead degraded.

A degradable bead may be useful in more quickly releasing an attached species (e.g., a nucleic acid molecule, a barcode sequence, a primer, etc) from the bead when the appropriate stimulus is applied to the bead as compared to a bead that does not degrade. For example, for a species bound to an inner surface of a porous bead or in the case of an encapsulated species, the species may have greater mobility and accessibility to other species in solution upon degradation of the bead. In some cases, a species may also be attached to a degradable bead via a degradable linker (e.g., disulfide linker). The degradable linker may respond to the same stimuli as the degradable bead or the two degradable species may respond to different stimuli. For example, a barcode sequence may be attached, via a disulfide bond, to a polyacrylamide bead comprising cystamine. Upon exposure of the barcoded-bead to a reducing agent, the bead degrades and the barcode sequence is released upon breakage of both the disulfide linkage between the barcode sequence and the bead and the disulfide linkages of the cystamine in the bead.

As will be appreciated from the above disclosure, while referred to as degradation of a bead, in many instances as noted above, that degradation may refer to the disassociation of a bound or entrained species from a bead, both with and without structurally degrading the physical bead itself. For example, entrained species may be released from beads through osmotic pressure differences due to, for example, changing chemical environments. By way of example, alteration of bead pore sizes due to osmotic pressure differences can generally occur without structural degradation of the bead itself In some cases, an increase in pore size due to osmotic swelling of a bead can permit the release of entrained species within the bead. In other cases, osmotic shrinking of a bead may cause a bead to better retain an entrained species due to pore size contraction.

Where degradable beads are provided, it may be beneficial to avoid exposing such beads to the stimulus or stimuli that cause such degradation prior to a given time, in order to, for example, avoid premature bead degradation and issues that arise from such degradation, including for example poor flow characteristics and aggregation. By way of example, where beads comprise reducible cross-linking groups, such as disulfide groups, it will be desirable to avoid contacting such beads with reducing agents, e.g., DTT or other disulfide cleaving reagents. In such cases, treatment to the beads described herein will, in some cases be provided free of reducing agents, such as DTT. Because reducing agents are often provided in commercial enzyme preparations, it may be desirable to provide reducing agent free (or DTT free) enzyme preparations in treating the beads described herein. Examples of such enzymes include, e.g., polymerase enzyme preparations, reverse transcriptase enzyme preparations, ligase enzyme preparations, as well as many other enzyme preparations that may be used to treat the beads described herein. The terms “reducing agent free” or “DTT free” preparations can refer to a preparation having less than about 1/10th, less than about 1/50th, or even less than about 1/100th of the lower ranges for such materials used in degrading the beads. For example, for DTT, the reducing agent free preparation can have less than about 0.01 millimolar (mM), 0.005 mM, 0.001 mM DTT, 0.0005 mM DTT, or even less than about 0.0001 mM DTT. In many cases, the amount of DTT can be undetectable.

Numerous chemical triggers may be used to trigger the degradation of beads. Examples of these chemical changes may include, but are not limited to pH-mediated changes to the integrity of a component within the bead, degradation of a component of a bead via cleavage of cross-linked bonds, and depolymerization of a component of a bead.

In some cases, a bead may be formed from materials that comprise degradable chemical crosslinkers, such as BAC or cystamine. Degradation of such degradable crosslinkers may be accomplished through a number of mechanisms. In some examples, a bead may be contacted with a chemical degrading agent that may induce oxidation, reduction or other chemical changes. For example, a chemical degrading agent may be a reducing agent, such as dithiothreitol (DTT). Additional examples of reducing agents may include β-mercaptoethanol, (2S)-2-amino-1,4-dimercaptobutane (dithiobutylamine or DTBA), tris(2-carboxyethyl) phosphine (TCEP), or combinations thereof. A reducing agent may degrade the disulfide bonds formed between gel precursors forming the bead, and thus, degrade the bead. In other cases, a change in pH of a solution, such as an increase in pH, may trigger degradation of a bead. In other cases, exposure to an aqueous solution, such as water, may trigger hydrolytic degradation, and thus degradation of the bead. In some cases, any combination of stimuli may trigger degradation of a bead. For example, a change in pH may enable a chemical agent (e.g., DTT) to become an effective reducing agent.

Beads may also be induced to release their contents upon the application of a thermal stimulus. A change in temperature can cause a variety of changes to a bead. For example, heat can cause a solid bead to liquefy. A change in heat may cause melting of a bead such that a portion of the bead degrades. In other cases, heat may increase the internal pressure of the bead components such that the bead ruptures or explodes. Heat may also act upon heat-sensitive polymers used as materials to construct beads.

Any suitable agent may degrade beads. In some cases, changes in temperature or pH may be used to degrade thermo-sensitive or pH-sensitive bonds within beads. In some cases, chemical degrading agents may be used to degrade chemical bonds within beads by oxidation, reduction or other chemical changes. For example, a chemical degrading agent may be a reducing agent, such as DTT, wherein DTT may degrade the disulfide bonds formed between a crosslinker and gel precursors, thus degrading the bead. In some cases, a reducing agent may be added to degrade the bead, which may or may not cause the bead to release its contents. Examples of reducing agents may include dithiothreitol (DTT), β-mercaptoethanol, (2S)-2-amino-1,4-dimercaptobutane (dithiobutylamine or DTBA), tris(2-carboxyethyl) phosphine (TCEP), or combinations thereof. The reducing agent may be present at a concentration of about 0.1 mM, 0.5 mM, 1 mM, 5 mM, 10 mM. The reducing agent may be present at a concentration of at least about 0.1 mM, 0.5 mM, 1 mM, 5 mM, 10 mM, or greater than 10 mM. The reducing agent may be present at concentration of at most about 10 mM, 5 mM, 1 mM, 0.5 mM, 0.1 mM, or less.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing oligonucleotide bearing beads.

Although FIG. 1 and FIG. 2 have been described in terms of providing substantially singly occupied partitions, above, in certain cases, it may be desirable to provide multiply occupied partitions, e.g., containing two, three, four or more cells and/or microcapsules (e.g., beads) comprising barcoded nucleic acid molecules (e.g., oligonucleotides) within a single partition. Accordingly, as noted above, the flow characteristics of the biological particle and/or bead containing fluids and partitioning fluids may be controlled to provide for such multiply occupied partitions. In particular, the flow parameters may be controlled to provide a given occupancy rate at greater than about 50% of the partitions, greater than about 75%, and in some cases greater than about 80%, 90%, 95%, or higher.

In some cases, additional microcapsules can be used to deliver additional reagents to a partition. In such cases, it may be advantageous to introduce different beads into a common channel or droplet generation junction, from different bead sources (e.g., containing different associated reagents) through different channel inlets into such common channel or droplet generation junction (e.g., junction 210). In such cases, the flow and frequency of the different beads into the channel or junction may be controlled to provide for a certain ratio of microcapsules from each source, while ensuring a given pairing or combination of such beads into a partition with a given number of biological particles (e.g., one biological particle and one bead per partition).

The partitions described herein may comprise small volumes, for example, less than about 10 microliters (μL), 5 μL, 1 μL, 900 picoliters (pL), 800 pL, 700 pL, 600 pL, 500 pL, 400pL, 300 pL, 200 pL, 100 pL, 50 pL, 20 pL, 10 pL, 1 pL, 500 nanoliters (nL), 100 nL, 50 nL, or less.

For example, in the case of droplet based partitions, the droplets may have overall volumes that are less than about 1000 pL, 900 pL, 800 pL, 700 pL, 600 pL, 500 pL, 400pL, 300 pL, 200 pL, 100pL, 50 pL, 20 pL, 10 pL, 1 pL, or less. Where co-partitioned with microcapsules, it will be appreciated that the sample fluid volume, e.g., including co-partitioned biological particles and/or beads, within the partitions may be less than about 90% of the above described volumes, less than about 80%, less than about 70%, less than about 60%, less than about 50%, less than about 40%, less than about 30%, less than about 20%, or less than about 10% of the above described volumes.

As is described elsewhere herein, partitioning species may generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions may comprise both unoccupied partitions (e.g., empty partitions) and occupied partitions.

### Reagents

In accordance with certain aspects, biological particles may be partitioned along with lysis reagents in order to release the contents of the biological particles within the partition. In such cases, the lysis agents can be contacted with the biological particle suspension concurrently with, or immediately prior to, the introduction of the biological particles into the partitioning junction/droplet generation zone (e.g., junction 210), such as through an additional channel or channels upstream of the channel junction. In accordance with other aspects, additionally or alternatively, biological particles may be partitioned along with other reagents, as will be described further below.

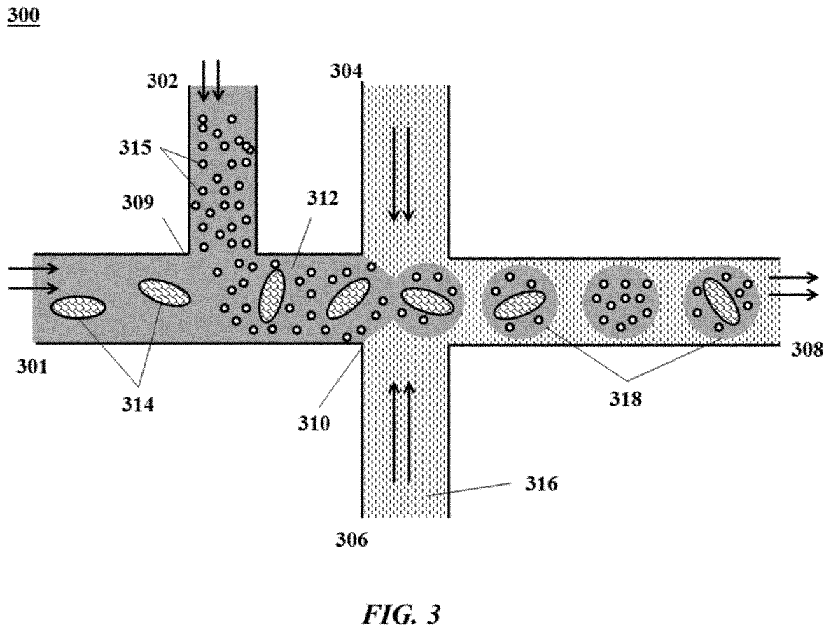

FIG. 3 shows an example of a microfluidic channel structure 300 for co-partitioning biological particles and reagents. The channel structure 300 can include channel segments 301, 302, 304, 306 and 308. Channel segments 301 and 302 communicate at a first channel junction 309. Channel segments 302, 304, 306, and 308 communicate at a second channel junction 310.

In an example operation, the channel segment 301 may transport an aqueous fluid 312 that includes a plurality of biological particles 314 along the channel segment 301 into the second junction 310. As an alternative or in addition to, channel segment 301 may transport beads (e.g., gel beads). The beads may comprise barcode molecules.

For example, the channel segment 301 may be connected to a reservoir comprising an aqueous suspension of biological particles 314. Upstream of, and immediately prior to reaching, the second junction 310, the channel segment 301 may meet the channel segment 302 at the first junction 309. The channel segment 302 may transport a plurality of reagents 315 (e.g., lysis agents) suspended in the aqueous fluid 312 along the channel segment 302 into the first junction 309. For example, the channel segment 302 may be connected to a reservoir comprising the reagents 315. After the first junction 309, the aqueous fluid 312 in the channel segment 301 can carry both the biological particles 314 and the reagents 315 towards the second junction 310. In some instances, the aqueous fluid 312 in the channel segment 301 can include one or more reagents, which can be the same or different reagents as the reagents 315. A second fluid 316 that is immiscible with the aqueous fluid 312 (e.g., oil) can be delivered to the second junction 310 from each of channel segments 304 and 306. Upon meeting of the aqueous fluid 312 from the channel segment 301 and the second fluid 316 from each of channel segments 304 and 306 at the second channel junction 310, the aqueous fluid 312 can be partitioned as discrete droplets 318 in the second fluid 316 and flow away from the second junction 310 along channel segment 308. The channel segment 308 may deliver the discrete droplets 318 to an outlet reservoir fluidly coupled to the channel segment 308, where they may be harvested.

The second fluid 316 can comprise an oil, such as a fluorinated oil, that includes a fluorosurfactant for stabilizing the resulting droplets, for example, inhibiting subsequent coalescence of the resulting droplets 318.

A discrete droplet generated may include an individual biological particle 314 and/or one or more reagents 315. In some instances, a discrete droplet generated may include a barcode carrying bead (not shown), such as via other microfluidics structures described elsewhere herein. In some instances, a discrete droplet may be unoccupied (e.g., no reagents, no biological particles).

Beneficially, when lysis reagents and biological particles are co-partitioned, the lysis reagents can facilitate the release of the contents of the biological particles within the partition. The contents released in a partition may remain discrete from the contents of other partitions.

As will be appreciated, the channel segments described herein may be coupled to any of a variety of different fluid sources or receiving components, including reservoirs, tubing, manifolds, or fluidic components of other systems. As will be appreciated, the microfluidic channel structure 300 may have other geometries. For example, a microfluidic channel structure can have more than two channel junctions. For example, a microfluidic channel structure can have 2, 3, 4, 5 channel segments or more each carrying the same or different types of beads, reagents, and/or biological particles that meet at a channel junction. Fluid flow in each channel segment may be controlled to control the partitioning of the different elements into droplets. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, Mo.), as well as other commercially available lysis enzymes. Other lysis agents may additionally or alternatively be co-partitioned with the biological particles to cause the release of the biological particle's contents into the partitions. For example, in some cases, surfactant-based lysis solutions may be used to lyse cells, although these may be less desirable for emulsion based systems where the surfactants can interfere with stable emulsions. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g., non-emulsion based partitioning such as encapsulation of biological particles that may be in addition to or in place of droplet partitioning, where any pore size of the encapsulate is sufficiently small to retain nucleic acid fragments of a given size, following cellular disruption.

Alternatively or in addition to the lysis agents co-partitioned with the biological particles described above, other reagents can also be co-partitioned with the biological particles, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated biological particles, the biological particles may be exposed to an appropriate stimulus to release the biological particles or their contents from a co-partitioned microcapsule. For example, in some cases, a chemical stimulus may be co-partitioned along with an encapsulated biological particle to allow for the degradation of the microcapsule and release of the cell or its contents into the larger partition. In some cases, this stimulus may be the same as the stimulus described elsewhere herein for release of nucleic acid molecules (e.g., oligonucleotides) from their respective microcapsule (e.g., bead). In alternative aspects, this may be a different and non-overlapping stimulus, in order to allow an encapsulated biological particle to be released into a partition at a different time from the release of nucleic acid molecules into the same partition.

Additional reagents may also be co-partitioned with the biological particles, such as endonucleases to fragment a biological particle's DNA, DNA polymerase enzymes and dNTPs used to amplify the biological particle's nucleic acid fragments and to attach the barcode molecular tags to the amplified fragments. Other enzymes may be co-partitioned, including without limitation, polymerase, transposase, ligase, proteinase K, DNAse, etc. Additional reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching. In some cases, template switching can be used to increase the length of a cDNA. In some cases, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner. Switch oligos can include sequences complementary to the additional nucleotides, e.g., polyG. The additional nucleotides (e.g., polyC) on the cDNA can hybridize to the additional nucleotides (e.g., polyG) on the switch oligo, whereby the switch oligo can be used by the reverse transcriptase as template to further extend the cDNA. Template switching oligonucleotides may comprise a hybridization region and a template region. The hybridization region can comprise any sequence capable of hybridizing to the target. In some cases, as previously described, the hybridization region comprises a series of G bases to complement the overhanging C bases at the 3′ end of a cDNA molecule. The series of G bases may comprise 1 G base, 2 G bases, 3 G bases, 4 G bases, 5 G bases or more than 5 G bases. The template sequence can comprise any sequence to be incorporated into the cDNA. In some cases, the template region comprises at least 1 (e.g., at least 2, 3, 4, 5 or more) tag sequences and/or functional sequences. Switch oligos may comprise deoxyribonucleic acids; ribonucleic acids; modified nucleic acids including 2-Aminopurine, 2,6-Diaminopurine (2-Amino-dA), inverted dT, 5-Methyl dC, 2′-deoxyInosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), or any combination.

In some cases, the length of a switch oligo may be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197 , 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides or longer.

In some cases, the length of a switch oligo may be at most about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197 , 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides.

Once the contents of the cells are released into their respective partitions, the macromolecular components (e.g., intracellular analytes of biological particles, such as RNA, DNA, or proteins) contained therein may be further processed within the partitions. In accordance with the methods and systems described herein, the macromolecular component contents of individual biological particles can be provided with unique identifiers such that, upon characterization of those macromolecular components they may be attributed as having been derived from the same biological particle or particles. The ability to attribute characteristics to individual biological particles or groups of biological particles is provided by the assignment of unique identifiers specifically to an individual biological particle or groups of biological particles. Unique identifiers, e.g., in the form of nucleic acid barcodes can be assigned or associated with individual biological particles or populations of biological particles, in order to tag or label the biological particle's macromolecular components (and as a result, its characteristics) with the unique identifiers. These unique identifiers can then be used to attribute the biological particle's components and characteristics to an individual biological particle or group of biological particles.

In some aspects, this is performed by co-partitioning the individual biological particle or groups of biological particles with the unique identifiers, such as described above (with reference to FIG. 2). In some aspects, the unique identifiers are provided in the form of nucleic acid molecules (e.g., oligonucleotides) that comprise nucleic acid barcode sequences that may be attached to or otherwise associated with the nucleic acid contents of individual biological particle, or to other components of the biological particle, and particularly to fragments of those nucleic acids. The nucleic acid molecules are partitioned such that as between nucleic acid molecules in a given partition, the nucleic acid barcode sequences contained therein are the same, but as between different partitions, the nucleic acid molecule can, and do have differing barcode sequences, or at least represent a large number of different barcode sequences across all of the partitions in a given analysis. In some aspects, only one nucleic acid barcode sequence can be associated with a given partition, although in some cases, two or more different barcode sequences may be present.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

The co-partitioned nucleic acid molecules can also comprise other functional sequences useful in the processing of the nucleic acids from the co-partitioned biological particles. These sequences include, e.g., targeted or random/universal amplification primer sequences for amplifying the genomic DNA from the individual biological particles within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning oligonucleotides may also be employed, including, e.g., coalescence of two or more droplets, where one droplet contains oligonucleotides, or microdispensing of oligonucleotides into partitions, e.g., droplets within microfluidic systems.

In an example, microcapsules, such as beads, are provided that each include large numbers of the above described barcoded nucleic acid molecules (e.g., barcoded oligonucleotides) releasably attached to the beads, where all of the nucleic acid molecules attached to a particular bead will include the same nucleic acid barcode sequence, but where a large number of diverse barcode sequences are represented across the population of beads used. In some cases, hydrogel beads, e.g., comprising polyacrylamide polymer matrices, are used as a solid support and delivery vehicle for the nucleic acid molecules into the partitions, as they are capable of carrying large numbers of nucleic acid molecules, and may be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. Additionally, each bead can be provided with large numbers of nucleic acid (e.g., oligonucleotide) molecules attached. In particular, the number of molecules of nucleic acid molecules including the barcode sequence on an individual bead can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules, or more. Nucleic acid molecules of a given bead can include identical (or common) barcode sequences, different barcode sequences, or a combination of both. Nucleic acid molecules of a given bead can include multiple sets of nucleic acid molecules. Nucleic acid molecules of a given set can include identical barcode sequences. The identical barcode sequences can be different from barcode sequences of nucleic acid molecules of another set.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules.

In some cases, it may be desirable to incorporate multiple different barcodes within a given partition, either attached to a single or multiple beads within the partition. For example, in some cases, a mixed, but known set of barcode sequences may provide greater assurance of identification in the subsequent processing, e.g., by providing a stronger address or attribution of the barcodes to a given partition, as a duplicate or independent confirmation of the output from a given partition.

The nucleic acid molecules (e.g., oligonucleotides) are releasable from the beads upon the application of a particular stimulus to the beads. In some cases, the stimulus may be a photo-stimulus, e.g., through cleavage of a photo-labile linkage that releases the nucleic acid molecules. In other cases, a thermal stimulus may be used, where elevation of the temperature of the beads environment will result in cleavage of a linkage or other release of the nucleic acid molecules from the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological particles, and may be degraded for release of the attached nucleic acid molecules through exposure to a reducing agent, such as DTT.

In some aspects, provided are systems and methods for controlled partitioning. Droplet size may be controlled by adjusting certain geometric features in channel architecture (e.g., microfluidics channel architecture). For example, an expansion angle, width, and/or length of a channel may be adjusted to control droplet size.

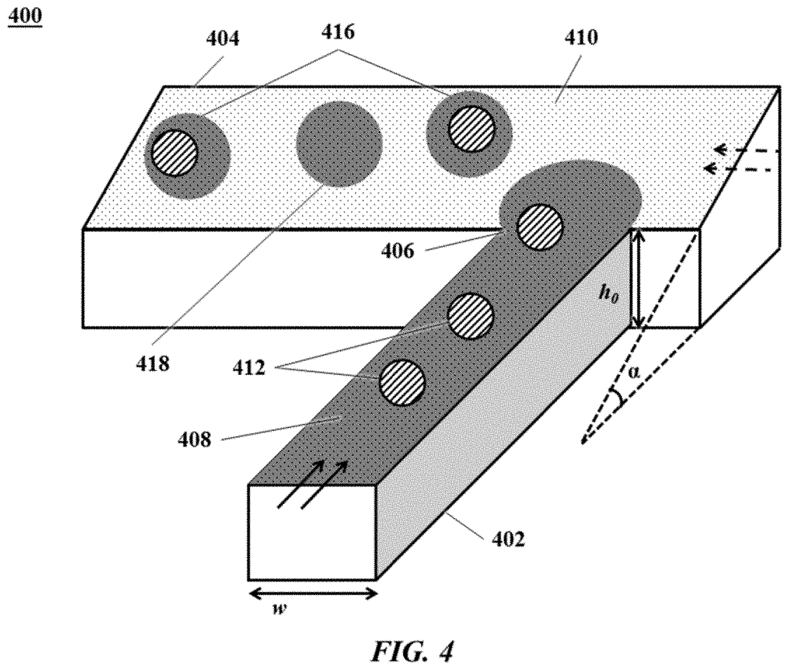

FIG. 4 shows an example of a microfluidic channel structure for the controlled partitioning of beads into discrete droplets. A channel structure 400 can include a channel segment 402 communicating at a channel junction 406 (or intersection) with a reservoir 404. The reservoir 404 can be a chamber. Any reference to “reservoir,” as used herein, can also refer to a “chamber.” In operation, an aqueous fluid 408 that includes suspended beads 412 may be transported along the channel segment 402 into the junction 406 to meet a second fluid 410 that is immiscible with the aqueous fluid 408 in the reservoir 404 to create droplets 416, 418 of the aqueous fluid 408 flowing into the reservoir 404. At the junction 406 where the aqueous fluid 408 and the second fluid 410 meet, droplets can form based on factors such as the hydrodynamic forces at the junction 406, flow rates of the two fluids 408, 410, fluid properties, and certain geometric parameters (e.g., w, h₀, α, etc.) of the channel structure 400. A plurality of droplets can be collected in the reservoir 404 by continuously injecting the aqueous fluid 408 from the channel segment 402 through the junction 406.

A discrete droplet generated may include a bead (e.g., as in occupied droplets 416). Alternatively, a discrete droplet generated may include more than one bead. Alternatively, a discrete droplet generated may not include any beads (e.g., as in unoccupied droplet 418). In some instances, a discrete droplet generated may contain one or more biological particles, as described elsewhere herein. In some instances, a discrete droplet generated may comprise one or more reagents, as described elsewhere herein.

In some instances, the aqueous fluid 408 can have a substantially uniform concentration or frequency of beads 412. The beads 412 can be introduced into the channel segment 402 from a separate channel (not shown in FIG. 4). The frequency of beads 412 in the channel segment 402 may be controlled by controlling the frequency in which the beads 412 are introduced into the channel segment 402 and/or the relative flow rates of the fluids in the channel segment 402 and the separate channel. In some instances, the beads can be introduced into the channel segment 402 from a plurality of different channels, and the frequency controlled accordingly.

In some instances, the aqueous fluid 408 in the channel segment 402 can comprise biological particles (e.g., described with reference to FIGS. 1 and 2). In some instances, the aqueous fluid 408 can have a substantially uniform concentration or frequency of biological particles. As with the beads, the biological particles can be introduced into the channel segment 402 from a separate channel. The frequency or concentration of the biological particles in the aqueous fluid 408 in the channel segment 402 may be controlled by controlling the frequency in which the biological particles are introduced into the channel segment 402 and/or the relative flow rates of the fluids in the channel segment 402 and the separate channel. In some instances, the biological particles can be introduced into the channel segment 402 from a plurality of different channels, and the frequency controlled accordingly. In some instances, a first separate channel can introduce beads and a second separate channel can introduce biological particles into the channel segment 402. The first separate channel introducing the beads may be upstream or downstream of the second separate channel introducing the biological particles.

The second fluid 410 can comprise an oil, such as a fluorinated oil, that includes a fluorosurfactant for stabilizing the resulting droplets, for example, inhibiting subsequent coalescence of the resulting droplets.

In some instances, the second fluid 410 may not be subjected to and/or directed to any flow in or out of the reservoir 404. For example, the second fluid 410 may be substantially stationary in the reservoir 404. In some instances, the second fluid 410 may be subjected to flow within the reservoir 404, but not in or out of the reservoir 404, such as via application of pressure to the reservoir 404 and/or as affected by the incoming flow of the aqueous fluid 408 at the junction 406. Alternatively, the second fluid 410 may be subjected and/or directed to flow in or out of the reservoir 404. For example, the reservoir 404 can be a channel directing the second fluid 410 from upstream to downstream, transporting the generated droplets.

The channel structure 400 at or near the junction 406 may have certain geometric features that at least partly determine the sizes of the droplets formed by the channel structure 400. The channel segment 402 can have a height, h₀ and width, w, at or near the junction 406. By way of example, the channel segment 402 can comprise a rectangular cross-section that leads to a reservoir 404 having a wider cross-section (such as in width or diameter). Alternatively, the cross-section of the channel segment 402 can be other shapes, such as a circular shape, trapezoidal shape, polygonal shape, or any other shapes. The top and bottom walls of the reservoir 404 at or near the junction 406 can be inclined at an expansion angle, α. The expansion angle, α, allows the tongue (portion of the aqueous fluid 408 leaving channel segment 402 at junction 406 and entering the reservoir 404 before droplet formation) to increase in depth and facilitate decrease in curvature of the intermediately formed droplet. Droplet size may decrease with increasing expansion angle. The resulting droplet radius, R_{d}, may be predicted by the following equation for the aforementioned geometric parameters of h₀, w, and α:

$${R}_{d} \approx 0.44 ( 1 + 2.2 \sqrt{tan α} \frac{w}{{h}_{0}} ) \frac{{h}_{0}}{\sqrt{tan α}}$$

By way of example, for a channel structure with w=21 μm, h=21 μm, and α=3°, the predicted droplet size is 121 μm. In another example, for a channel structure with w=25 μm, h=25 μm, and α=5°, the predicted droplet size is 123 μm. In another example, for a channel structure with w=28 μm, h=28 μm, and α=7°, the predicted droplet size is 124 μm.

In some instances, the expansion angle, α, may be between a range of from about 0.5° to about 4°, from about 0.1° to about 10°, or from about 0° to about 90°. For example, the expansion angle can be at least about 0.01°, 0.1°, 0.2°, 0.3°, 0.4°, 0.5°, 0.6°, 0.7°, 0.8°, 0.9°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85°, or higher.

In some instances, the expansion angle can be at most about 89°, 88°, 87°, 86°, 85°, 84°, 83°, 82°, 81°, 80°, 75°, 70°, 65°, 60°, 55°, 50°, 45°, 40°, 35°, 30°, 25°, 20°, 15°, 10°, 9°, 8°, 7°, 6°, 5°, 4°, 3°, 2°, 1°, 0.1°, 0.01°, or less. In some instances, the width, w, can be between a range of from about 100 micrometers (μm) to about 500 μm. In some instances, the width, w, can be between a range of from about 10 μm to about 200 μm. Alternatively, the width can be less than about 10 μm. Alternatively, the width can be greater than about 500 μm. In some instances, the flow rate of the aqueous fluid 408 entering the junction 406 can be between about 0.04 microliters (μL)/minute (min) and about 40 μL/min. In some instances, the flow rate of the aqueous fluid 408 entering the junction 406 can be between about 0.01 microliters (μL)/minute (min) and about 100 μL/min. Alternatively, the flow rate of the aqueous fluid 408 entering the junction 406 can be less than about 0.01 μL/min. Alternatively, the flow rate of the aqueous fluid 408 entering the junction 406 can be greater than about 40 μL/min, such as 45 μL/min, 50 μL/min, 55 μL/min, 60 μL/min, 65 μL/min, 70 μL/min, 75 μL/min, 80 μL/min, 85 μL/min, 90 μL/min, 95 μL/min, 100 μL/min, 110 μL/min, 120 μL/min, 130 μL/min, 140 μL/min, 150 μL/min, or greater. At lower flow rates, such as flow rates of about less than or equal to 10 microliters/minute, the droplet radius may not be dependent on the flow rate of the aqueous fluid 408 entering the junction 406.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

The throughput of droplet generation can be increased by increasing the points of generation, such as increasing the number of junctions (e.g., junction 406) between aqueous fluid 408 channel segments (e.g., channel segment 402) and the reservoir 404. Alternatively or in addition, the throughput of droplet generation can be increased by increasing the flow rate of the aqueous fluid 408 in the channel segment 402.

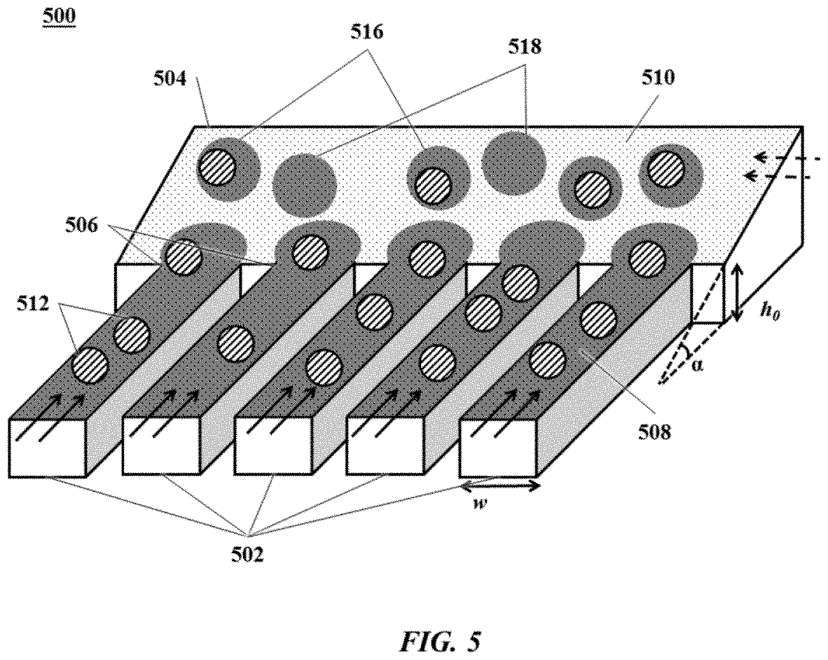

FIG. 5 shows an example of a microfluidic channel structure for increased droplet generation throughput. A microfluidic channel structure 500 can comprise a plurality of channel segments 502 and a reservoir 504. Each of the plurality of channel segments 502 may be in fluid communication with the reservoir 504. The channel structure 500 can comprise a plurality of channel junctions 506 between the plurality of channel segments 502 and the reservoir 504. Each channel junction can be a point of droplet generation. The channel segment 402 from the channel structure 400 in FIG. 4 and any description to the components thereof may correspond to a given channel segment of the plurality of channel segments 502 in channel structure 500 and any description to the corresponding components thereof. The reservoir 404 from the channel structure 400 and any description to the components thereof may correspond to the reservoir 504 from the channel structure 500 and any description to the corresponding components thereof.

Each channel segment of the plurality of channel segments 502 may comprise an aqueous fluid 508 that includes suspended beads 512. The reservoir 504 may comprise a second fluid 510 that is immiscible with the aqueous fluid 508. In some instances, the second fluid 510 may not be subjected to and/or directed to any flow in or out of the reservoir 504. For example, the second fluid 510 may be substantially stationary in the reservoir 504. In some instances, the second fluid 510 may be subjected to flow within the reservoir 504, but not in or out of the reservoir 504, such as via application of pressure to the reservoir 504 and/or as affected by the incoming flow of the aqueous fluid 508 at the junctions. Alternatively, the second fluid 510 may be subjected and/or directed to flow in or out of the reservoir 504. For example, the reservoir 504 can be a channel directing the second fluid 510 from upstream to downstream, transporting the generated droplets.

In operation, the aqueous fluid 508 that includes suspended beads 512 may be transported along the plurality of channel segments 502 into the plurality of junctions 506 to meet the second fluid 510 in the reservoir 504 to create droplets 516, 518. A droplet may form from each channel segment at each corresponding junction with the reservoir 504. At the junction where the aqueous fluid 508 and the second fluid 510 meet, droplets can form based on factors such as the hydrodynamic forces at the junction, flow rates of the two fluids 508, 510, fluid properties, and certain geometric parameters (e.g., w, ho, a, etc.) of the channel structure 500, as described elsewhere herein. A plurality of droplets can be collected in the reservoir 504 by continuously injecting the aqueous fluid 508 from the plurality of channel segments 502 through the plurality of junctions 506. Throughput may significantly increase with the parallel channel configuration of channel structure 500. For example, a channel structure having five inlet channel segments comprising the aqueous fluid 508 may generate droplets five times as frequently than a channel structure having one inlet channel segment, provided that the fluid flow rate in the channel segments are substantially the same. The fluid flow rate in the different inlet channel segments may or may not be substantially the same. A channel structure may have as many parallel channel segments as is practical and allowed for the size of the reservoir. For example, the channel structure may have at least about 2, 3, 4, 5, 6, 7, 8, 9, 10, 20, 30, 40, 50, 60, 70, 80, 90, 100, 150, 500, 250, 300, 350, 400, 450, 500, 600, 700, 800, 900, 1000, 1500, 5000 or more parallel or substantially parallel channel segments.

The geometric parameters, w, h₀, and α, may or may not be uniform for each of the channel segments in the plurality of channel segments 502. For example, each channel segment may have the same or different widths at or near its respective channel junction with the reservoir 504. For example, each channel segment may have the same or different height at or near its respective channel junction with the reservoir 504. In another example, the reservoir 504 may have the same or different expansion angle at the different channel junctions with the plurality of channel segments 502. When the geometric parameters are uniform, beneficially, droplet size may also be controlled to be uniform even with the increased throughput. In some instances, when it is desirable to have a different distribution of droplet sizes, the geometric parameters for the plurality of channel segments 502 may be varied accordingly.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

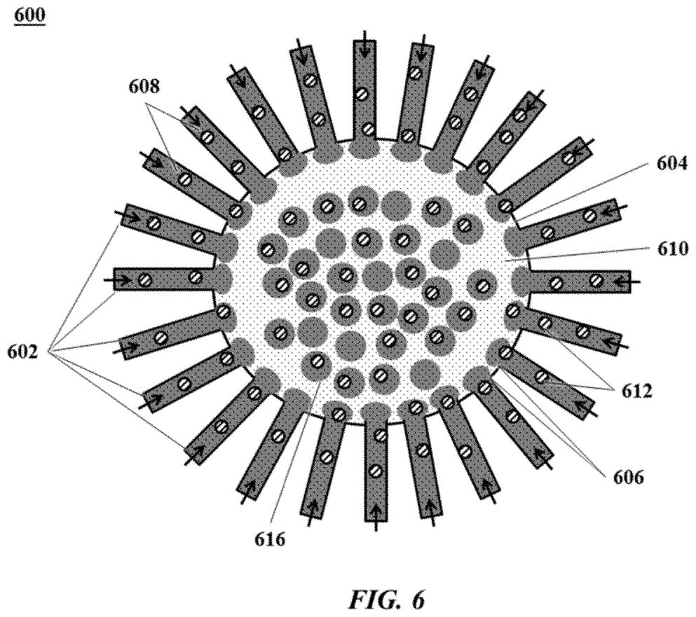

FIG. 6 shows another example of a microfluidic channel structure for increased droplet generation throughput. A microfluidic channel structure 600 can comprise a plurality of channel segments 602 arranged generally circularly around the perimeter of a reservoir 604. Each of the plurality of channel segments 602 may be in fluid communication with the reservoir 604. The channel structure 600 can comprise a plurality of channel junctions 606 between the plurality of channel segments 602 and the reservoir 604. Each channel junction can be a point of droplet generation. The channel segment 402 from the channel structure 400 in FIG. 4 and any description to the components thereof may correspond to a given channel segment of the plurality of channel segments 602 in channel structure 600 and any description to the corresponding components thereof. The reservoir 404 from the channel structure 400 and any description to the components thereof may correspond to the reservoir 604 from the channel structure 600 and any description to the corresponding components thereof.

Each channel segment of the plurality of channel segments 602 may comprise an aqueous fluid 608 that includes suspended beads 612. The reservoir 604 may comprise a second fluid 610 that is immiscible with the aqueous fluid 608. In some instances, the second fluid 610 may not be subjected to and/or directed to any flow in or out of the reservoir 604. For example, the second fluid 610 may be substantially stationary in the reservoir 604. In some instances, the second fluid 610 may be subjected to flow within the reservoir 604, but not in or out of the reservoir 604, such as via application of pressure to the reservoir 604 and/or as affected by the incoming flow of the aqueous fluid 608 at the junctions. Alternatively, the second fluid 610 may be subjected and/or directed to flow in or out of the reservoir 604. For example, the reservoir 604 can be a channel directing the second fluid 610 from upstream to downstream, transporting the generated droplets.

In operation, the aqueous fluid 608 that includes suspended beads 612 may be transported along the plurality of channel segments 602 into the plurality of junctions 606 to meet the second fluid 610 in the reservoir 604 to create a plurality of droplets 616. A droplet may form from each channel segment at each corresponding junction with the reservoir 604. At the junction where the aqueous fluid 608 and the second fluid 610 meet, droplets can form based on factors such as the hydrodynamic forces at the junction, flow rates of the two fluids 608, 610, fluid properties, and certain geometric parameters (e.g., widths and heights of the channel segments 602, expansion angle of the reservoir 604, etc.) of the channel structure 600, as described elsewhere herein. A plurality of droplets can be collected in the reservoir 604 by continuously injecting the aqueous fluid 608 from the plurality of channel segments 602 through the plurality of junctions 606. Throughput may significantly increase with the substantially parallel channel configuration of the channel structure 600. A channel structure may have as many substantially parallel channel segments as is practical and allowed for by the size of the reservoir. For example, the channel structure may have at least about 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, 1000, 1500, 5000 or more parallel or substantially parallel channel segments. The plurality of channel segments may be substantially evenly spaced apart, for example, around an edge or perimeter of the reservoir. Alternatively, the spacing of the plurality of channel segments may be uneven.

The reservoir 604 may have an expansion angle, a (not shown in FIG. 6) at or near each channel junction. Each channel segment of the plurality of channel segments 602 may have a width, w, and a height, h₀, at or near the channel junction. The geometric parameters, w, h₀, and α, may or may not be uniform for each of the channel segments in the plurality of channel segments 602. For example, each channel segment may have the same or different widths at or near its respective channel junction with the reservoir 604. For example, each channel segment may have the same or different height at or near its respective channel junction with the reservoir 604.

The reservoir 604 may have the same or different expansion angle at the different channel junctions with the plurality of channel segments 602. For example, a circular reservoir (as shown in FIG. 6) may have a conical, dome-like, or hemispherical ceiling (e.g., top wall) to provide the same or substantially same expansion angle for each channel segments 602 at or near the plurality of channel junctions 606. When the geometric parameters are uniform, beneficially, resulting droplet size may be controlled to be uniform even with the increased throughput. In some instances, when it is desirable to have a different distribution of droplet sizes, the geometric parameters for the plurality of channel segments 602 may be varied accordingly.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size. The beads and/or biological particle injected into the droplets may or may not have uniform size.

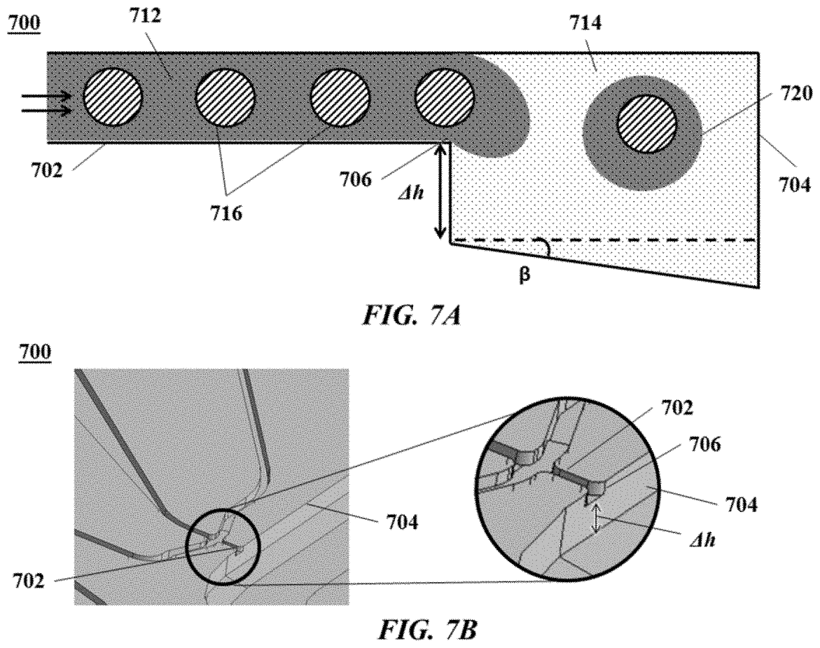

FIG. 7A shows a cross-section view of another example of a microfluidic channel structure with a geometric feature for controlled partitioning. A channel structure 700 can include a channel segment 702 communicating at a channel junction 706 (or intersection) with a reservoir 704. In some instances, the channel structure 700 and one or more of its components can correspond to the channel structure 100 and one or more of its components. FIG. 7B shows a perspective view of the channel structure 700 of FIG. 7A.

An aqueous fluid 712 comprising a plurality of particles 716 may be transported along the channel segment 702 into the junction 706 to meet a second fluid 714 (e.g., oil, etc.) that is immiscible with the aqueous fluid 712 in the reservoir 704 to create droplets 720 of the aqueous fluid 712 flowing into the reservoir 704. At the junction 706 where the aqueous fluid 712 and the second fluid 714 meet, droplets can form based on factors such as the hydrodynamic forces at the junction 706, relative flow rates of the two fluids 712, 714, fluid properties, and certain geometric parameters (e.g., Δh, etc.) of the channel structure 700. A plurality of droplets can be collected in the reservoir 704 by continuously injecting the aqueous fluid 712 from the channel segment 702 at the junction 706.

A discrete droplet generated may comprise one or more particles of the plurality of particles 716. As described elsewhere herein, a particle may be any particle, such as a bead, cell bead, gel bead, biological particle, intracellular analytes of biological particle, or other particles. Alternatively, a discrete droplet generated may not include any particles.

In some instances, the aqueous fluid 712 can have a substantially uniform concentration or frequency of particles 716. As described elsewhere herein (e.g., with reference to FIG. 4), the particles 716 (e.g., beads) can be introduced into the channel segment 702 from a separate channel (not shown in FIG. 7). The frequency of particles 716 in the channel segment 702 may be controlled by controlling the frequency in which the particles 716 are introduced into the channel segment 702 and/or the relative flow rates of the fluids in the channel segment 702 and the separate channel. In some instances, the particles 716 can be introduced into the channel segment 702 from a plurality of different channels, and the frequency controlled accordingly. In some instances, different particles may be introduced via separate channels. For example, a first separate channel can introduce beads and a second separate channel can introduce biological particles into the channel segment 702. The first separate channel introducing the beads may be upstream or downstream of the second separate channel introducing the biological particles.

In some instances, the second fluid 714 may not be subjected to and/or directed to any flow in or out of the reservoir 704. For example, the second fluid 714 may be substantially stationary in the reservoir 704. In some instances, the second fluid 714 may be subjected to flow within the reservoir 704, but not in or out of the reservoir 704, such as via application of pressure to the reservoir 704 and/or as affected by the incoming flow of the aqueous fluid 712 at the junction 706. Alternatively, the second fluid 714 may be subjected and/or directed to flow in or out of the reservoir 704. For example, the reservoir 704 can be a channel directing the second fluid 714 from upstream to downstream, transporting the generated droplets.

The channel structure 700 at or near the junction 706 may have certain geometric features that at least partly determine the sizes and/or shapes of the droplets formed by the channel structure 700. The channel segment 702 can have a first cross-section height, h₁, and the reservoir 704 can have a second cross-section height, h₂. The first cross-section height, h₁, and the second cross-section height, h₂, may be different, such that at the junction 706, there is a height difference of Δh. The second cross-section height, h₂, may be greater than the first cross-section height, h₁. In some instances, the reservoir may thereafter gradually increase in cross-section height, for example, the more distant it is from the junction 706. In some instances, the cross-section height of the reservoir may increase in accordance with expansion angle, β, at or near the junction 706. The height difference, Δh, and/or expansion angle, β, can allow the tongue (portion of the aqueous fluid 712 leaving channel segment 702 at junction 706 and entering the reservoir 704 before droplet formation) to increase in depth and facilitate decrease in curvature of the intermediately formed droplet. For example, droplet size may decrease with increasing height difference and/or increasing expansion angle.

The height difference, Δh, can be at least about 1 μm. Alternatively, the height difference can be at least about 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500 μm or more. Alternatively, the height difference can be at most about 500, 400, 300, 200, 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, 1 μm or less. In some instances, the expansion angle, β, may be between a range of from about 0.5° to about 4°, from about 0.1° to about 10°, or from about 0° to about 90°. For example, the expansion angle can be at least about 0.01°, 0.1°, 0.2°, 0.3°, 0.4°, 0.5°, 0.6°, 0.7°, 0.8°, 0.9°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85°, or higher. In some instances, the expansion angle can be at most about 89°, 88°, 87°, 86°, 85°, 84°, 83°, 82°, 81°, 80°, 75°, 70°, 65°, 60°, 55°, 50°, 45°, 40°, 35°, 30°, 25°, 20°, 15°, 10°, 9°, 8°, 7°, 6°, 5°, 4°, 3°, 2°, 1°, 0.1°, 0.01°, or less.

In some instances, the flow rate of the aqueous fluid 712 entering the junction 706 can be between about 0.04 microliters (μL)/minute (min) and about 40 μL/min. In some instances, the flow rate of the aqueous fluid 712 entering the junction 706 can be between about 0.01 microliters (μL)/minute (min) and about 100 μL/min. Alternatively, the flow rate of the aqueous fluid 712 entering the junction 706 can be less than about 0.01 μL/min. Alternatively, the flow rate of the aqueous fluid 712 entering the junction 706 can be greater than about 40 μL/min, such as 45 μL/min, 50 μL/min, 55 μL/min, 60 μL/min, 65 μL/min, 70 μL/min, 75 μL/min, 80 μL/min, 85 μL/min, 90 μL/min, 95 μL/min, 100 μL/min, 110 μL/min, 120 μL/min, 130 μL/min, 140 μL/min, 150 μL/min, or greater. At lower flow rates, such as flow rates of about less than or equal to 10 microliters/minute, the droplet radius may not be dependent on the flow rate of the aqueous fluid 712 entering the junction 706. The second fluid 714 may be stationary, or substantially stationary, in the reservoir 704. Alternatively, the second fluid 714 may be flowing, such as at the above flow rates described for the aqueous fluid 712.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

While FIGS. 7A and 7B illustrate the height difference, zlh, being abrupt at the junction 706 (e.g., a step increase), the height difference may increase gradually (e.g., from about 0 μm to a maximum height difference). Alternatively, the height difference may decrease gradually (e.g., taper) from a maximum height difference. A gradual increase or decrease in height difference, as used herein, may refer to a continuous incremental increase or decrease in height difference, wherein an angle between any one differential segment of a height profile and an immediately adjacent differential segment of the height profile is greater than 90°. For example, at the junction 706, a bottom wall of the channel and a bottom wall of the reservoir can meet at an angle greater than 90°. Alternatively or in addition, a top wall (e.g., ceiling) of the channel and a top wall (e.g., ceiling) of the reservoir can meet an angle greater than 90°. A gradual increase or decrease may be linear or non-linear (e.g., exponential, sinusoidal, etc.). Alternatively or in addition, the height difference may variably increase and/or decrease linearly or non-linearly. While FIGS. 7A and 7B illustrate the expanding reservoir cross-section height as linear (e.g., constant expansion angle, β), the cross-section height may expand non-linearly. For example, the reservoir may be defined at least partially by a dome-like (e.g., hemispherical) shape having variable expansion angles. The cross-section height may expand in any shape.

The channel networks, e.g., as described above or elsewhere herein, can be fluidly coupled to appropriate fluidic components. For example, the inlet channel segments are fluidly coupled to appropriate sources of the materials they are to deliver to a channel junction. These sources may include any of a variety of different fluidic components, from simple reservoirs defined in or connected to a body structure of a microfluidic device, to fluid conduits that deliver fluids from off-device sources, manifolds, fluid flow units (e.g., actuators, pumps, compressors) or the like. Likewise, the outlet channel segment (e.g., channel segment 208, reservoir 604, etc.) may be fluidly coupled to a receiving vessel or conduit for the partitioned cells for subsequent processing. Again, this may be a reservoir defined in the body of a microfluidic device, or it may be a fluidic conduit for delivering the partitioned cells to a subsequent process operation, instrument or component.

The methods and systems described herein may be used to greatly increase the efficiency of single cell applications and/or other applications receiving droplet-based input. For example, following the sorting of occupied cells and/or appropriately-sized cells, subsequent operations that can be performed can include generation of amplification products, purification (e.g., via solid phase reversible immobilization (SPRI)), further processing (e.g., shearing, ligation of functional sequences, and subsequent amplification (e.g., via PCR)). These operations may occur in bulk (e.g., outside the partition). In the case where a partition is a droplet in an emulsion, the emulsion can be broken and the contents of the droplet pooled for additional operations. Additional reagents that may be co-partitioned along with the barcode bearing bead may include oligonucleotides to block ribosomal RNA (rRNA) and nucleases to digest genomic DNA from cells. Alternatively, rRNA removal agents may be applied during additional processing operations. The configuration of the constructs generated by such a method can help minimize (or avoid) sequencing of the poly-T sequence during sequencing and/or sequence the 5′ end of a polynucleotide sequence. The amplification products, for example, first amplification products and/or second amplification products, may be subject to sequencing for sequence analysis. In some cases, amplification may be performed using the Partial Hairpin Amplification for Sequencing (PHASE) method.

A variety of applications require the evaluation of the presence and quantification of different biological particle or organism types within a population of biological particles, including, for example, microbiome analysis and characterization, environmental testing, food safety testing, epidemiological analysis, e.g., in tracing contamination or the like.

### Computer Systems

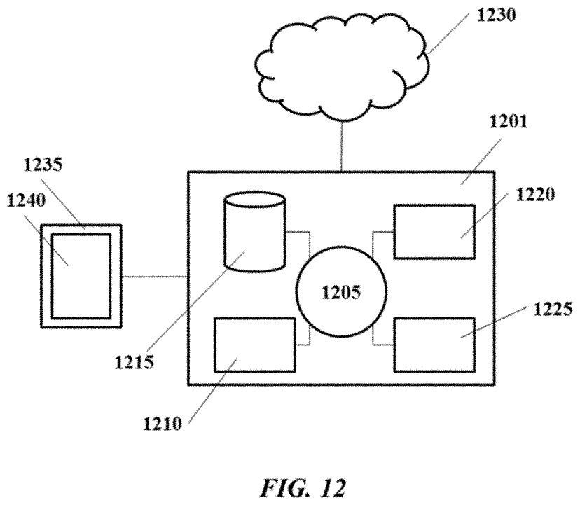

The present disclosure provides computer systems that are programmed to implement methods of the disclosure. FIG. 12 shows a computer system 1201 that is programmed or otherwise configured to maintain and regulate combinatorial barcode libraries described herein, process cells and/or cell beads, and control microfluidics systems. The computer system 1201 can regulate various aspects of the present disclosure. The computer system 1201 can be an electronic device of a user or a computer system that is remotely located with respect to the electronic device. The electronic device can be a mobile electronic device.

The computer system 1201 includes a central processing unit (CPU, also “processor” and “computer processor” herein) 1205, which can be a single core or multi core processor, or a plurality of processors for parallel processing. The computer system 1201 also includes memory or memory location 1210 (e.g., random-access memory, read-only memory, flash memory), electronic storage unit 1215 (e.g., hard disk), communication interface 1220 (e.g., network adapter) for communicating with one or more other systems, and peripheral devices 1225, such as cache, other memory, data storage and/or electronic display adapters. The memory 1210, storage unit 1215, interface 1220 and peripheral devices 1225 are in communication with the CPU 1205 through a communication bus (solid lines), such as a motherboard. The storage unit 1215 can be a data storage unit (or data repository) for storing data. The computer system 1201 can be operatively coupled to a computer network (“network”) 1230 with the aid of the communication interface 1220. The network 1230 can be the Internet, an internet and/or extranet, or an intranet and/or extranet that is in communication with the Internet. The network 1230 in some cases is a telecommunication and/or data network. The network 1230 can include one or more computer servers, which can enable distributed computing, such as cloud computing. The network 1230, in some cases with the aid of the computer system 1201, can implement a peer-to-peer network, which may enable devices coupled to the computer system 1201 to behave as a client or a server.

The CPU 1205 can execute a sequence of machine-readable instructions, which can be embodied in a program or software. The instructions may be stored in a memory location, such as the memory 1210. The instructions can be directed to the CPU 1205, which can subsequently program or otherwise configure the CPU 1205 to implement methods of the present disclosure. Examples of operations performed by the CPU 1205 can include fetch, decode, execute, and writeback.

The CPU 1205 can be part of a circuit, such as an integrated circuit. One or more other components of the system 1201 can be included in the circuit. In some cases, the circuit is an application specific integrated circuit (ASIC).

The storage unit 1215 can store files, such as drivers, libraries and saved programs. The storage unit 1215 can store user data, e.g., user preferences and user programs. The computer system 1201 in some cases can include one or more additional data storage units that are external to the computer system 1201, such as located on a remote server that is in communication with the computer system 1201 through an intranet or the Internet.

The computer system 1201 can communicate with one or more remote computer systems through the network 1230. For instance, the computer system 1201 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1201 via the network 1230.

Methods as described herein can be implemented by way of machine (e.g., computer processor) executable code stored on an electronic storage location of the computer system 1201, such as, for example, on the memory 1210 or electronic storage unit 1215. The machine executable or machine readable code can be provided in the form of software. During use, the code can be executed by the processor 1205. In some cases, the code can be retrieved from the storage unit 1215 and stored on the memory 1210 for ready access by the processor 1205. In some situations, the electronic storage unit 1215 can be precluded, and machine-executable instructions are stored on memory 1210.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 1201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1201 can include or be in communication with an electronic display 1235 that comprises a user interface (UI) 1240 for providing, for example, results or intermediary status of partitioning, barcoding, and/or downstream analysis (e.g., sequencing analysis). Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 1205.

Devices, systems, compositions and methods of the present disclosure may be used for various applications, such as, for example, processing a single analyte (e.g., RNA, DNA, or protein) or multiple analytes (e.g., DNA and RNA, DNA and protein, RNA and protein, or RNA, DNA and protein) from a single cell. For example, a biological particle (e.g., a cell or cell bead) is partitioned in a partition (e.g., droplet), and multiple analytes from the biological particle are processed for subsequent processing. The multiple analytes may be from the single cell. This may enable, for example, simultaneous proteomic, transcriptomic and genomic analysis of the cell.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-20. (canceled)

21. A method of processing a cell, comprising:
(a) providing:
(i) a labeled cell, wherein the labeled cell comprises (1) a nucleic acid molecule attached to a surface of the labeled cell and (2) a nucleic acid analyte,
(ii) a nucleic acid barcode sequence, and
(iii) a capture sequence, wherein the capture sequence is configured to capture the nucleic acid analyte; and
(b) attaching the nucleic acid barcode sequence and the capture sequence to the nucleic acid molecule, thereby generating a composite nucleic acid molecule comprising the nucleic acid barcode sequence and the capture sequence on the surface of the labeled cell.

22. The method of claim 21, wherein the nucleic acid analyte comprises a messenger ribonucleic acid (RNA) molecule, a clustered regularly interspaced short palindromic (CRISPR) RNA molecule, or a single guide RNA (sgRNA) molecule.

23. The method of claim 21, wherein the nucleic acid analyte comprises genomic deoxyribonucleic acid (DNA) molecule.

24. The method of claim 221, wherein the capture sequence comprises a poly-T sequence or a random N-mer sequence.

25. The method of claim 21, wherein the method comprises performing (a) and (b) in a droplet.

26. The method of claim 21, wherein the method comprises performing (a) and (b) in a well.

27. The method of claim 21, wherein (a) comprises providing an additional nucleic acid molecule comprising the nucleic acid barcode sequence and the capture sequence.

28. The method of claim 21, wherein the nucleic acid molecule is attached to the surface of the labeled cell through a cell coupling agent.

29. The method of claim 28, wherein the nucleic acid molecule is directly attached to the cell coupling agent.

30. The method of claim 28, wherein the nucleic acid molecule is indirectly attached to the cell coupling agent.

31. The method of claim 28, wherein the cell coupling agent is selected from the group consisting of an antibody, a lipophilic molecule, a saccharide, a protein, and a fluorophore.

32. The method of claim 21, wherein the nucleic acid molecule comprises one or more functional sequences selected from the group consisting of an adapter sequence, a primer, primer binding sequence, a unique molecular index (UMI) sequence, and a sequence configured to attach to the flow cell of a sequencer.

33. A method of processing a cell, comprising:
(a) providing a labeled cell, wherein the labeled cell comprises:
(i) a nucleic acid molecule attached to a surface of the labeled cell, and wherein the nucleic acid molecule comprises a nucleic acid barcode sequence and a capture sequence, wherein the capture sequence is configured to capture a nucleic acid analyte within the cell, and
(ii) the nucleic acid analyte; and
(b) generating a barcoded nucleic acid molecule using the nucleic acid molecule and the nucleic acid analyte, wherein the barcoded nucleic acid molecule comprises:
(i) a first sequence corresponding to the nucleic acid analyte, and
(ii) a second sequence corresponding to the nucleic acid barcode sequence.

34. The method of claim 33, wherein the nucleic acid analyte comprises a messenger ribonucleic acid (RNA) molecule, a clustered regularly interspaced short palindromic (CRISPR) RNA molecule, or a single guide RNA (sgRNA) molecule.

35. The method of claim 33, wherein the nucleic acid analyte comprises genomic deoxyribonucleic acid (DNA) molecule.

36. The method of claim 33, wherein the capture sequence comprises a poly-T sequence or a random N-mer sequence.

37. The method of claim 33, wherein the method comprises performing (a) and (b) in a droplet.

38. The method of claim 33, wherein the method comprises performing (a) and (b) in a well.

39. The method of claim 33, wherein generating the barcoded nucleic acid comprises hybridizing the capture sequence to the nucleic acid analyte and performing a nucleic acid extension reaction.

40. The method of claim 33, wherein the nucleic acid molecule is attached to the surface of the labeled cell through a cell coupling agent.

41. The method of claim 40, wherein the nucleic acid molecule is directly attached to the cell coupling agent.

42. The method of claim 40, wherein the nucleic acid molecule is indirectly attached to the cell coupling agent.

43. The method of claim 40, wherein the cell coupling agent is selected from the group consisting of an antibody, a lipophilic molecule, a saccharide, a protein, and a fluorophore.

44. The method of claim 33, wherein the nucleic acid molecule comprises one or more functional sequences selected from the group consisting of an adapter sequence, a primer, primer binding sequence, a unique molecular index (UMI) sequence, and a sequence configured to attach to the flow cell of a sequencer.

45. The method of claim 33, wherein the method comprises, before (a), attaching the nucleic acid molecule to the cell to generate the labeled cell.

46. A cell comprising:
(i) a nucleic acid molecule attached to the surface of the labeled cell, and wherein the nucleic acid molecule comprises a nucleic acid barcode sequence and a capture sequence, wherein the capture sequence configured to capture a nucleic acid analyte within the cell, and
(ii) the nucleic acid analyte.
